(19) United States
(12) Patent Application Publication
(10) Pub. No.: US 2024/0181078 A1
(43) Pub. Date: Jun. 6, 2024

(54) Simian Adenovirus and Hybrid Adenoviral Vectors

(71) Applicant: Oxford University Innovation Limited, Oxford (GB)

(72) Inventors: Matthew Douglas James Dicks, Oxford (GB); Matthew Guy Cottingham, Oxford (GB); Adrian Vivian Sinton Hill, Oxford (GB); Sarah Gilbert, Oxford (GB)

(73) Assignee: Oxford University Innovation Limited, Oxford (GB)

(21) Appl. No.: 18/511,357

(22) Filed: Nov. 16, 2023

Related U.S. Application Data
(63) Continuation of application No. 16/849,301, filed on Apr. 15, 2020, now Pat. No. 11,857,640.
Application No. 16/849,301 is a continuation of application No. 15/628,277, filed on Jun. 20, 2017, now Pat. No. 10,646,587.
Application No. 15/628,277 is a continuation of application No. 14/119,413, filed on Mar. 19, 2014, now Pat. No. 9,714,435.

(30) Foreign Application Priority Data
May 25, 2011 (GB) 1108879.6

(51) Int. Cl. A61K 48/00; C12N 5/0735; C12N 7/00; C12N 15/86

(52) U.S. Cl. CPC A61K 48/00; C12N 5/0606; C12N 7/00; C12N 15/86; C12N 2710/10021; C12N 2710/10042; C12N 2710/10044; C12N 2800/204

(57) ABSTRACT

Recombinant adenoviral vectors, immunogenic compositions thereof and their use in medicine, and methods for generating recombinant adenoviral vectors are provided. In particular, the an adenovirus vector having a capsid derived from chimpanzee adenovirus AdY25, wherein the capsid encapsidates a nucleic acid molecule comprising an exogeneous nucleotide sequence of interest are provided.

### RELATED APPLICATIONS

This application is a continuation of U.S. application Ser. No. 16/849,301 filed 15 Apr. 2020, which is a continuation of U.S. application Ser. No. 15/628,277 filed 20 Jun. 2017, now U.S. Pat. No. 10,646,587, that in turn is a continuation of U.S. application Ser. No. 14/119,413 filed 19 Mar. 2014, now U.S. Pat. No. 9,714,435, that in turn is a U.S. national phase application of PCT/GB2012/000467 filed 25 May 2012, that in turn claims priority benefit of GB 1108879.6 filed 25 May 2011; the contents of all of which are hereby incorporated by reference.

### SEQUENCE LISTING

The instant application contains a Sequence Listing which has been submitted electronically in XML format and is hereby incorporated by reference in its entirety. Said XML copy, created on Feb. 23, 2024, is named 119887-5003-US02 SEQ LIST.xml which is 149 KB in size.

### FIELD OF THE INVENTION

The present invention relates to novel adenoviral vectors derived from a chimpanzee adenovirus, immunogenic compositions thereof and their use in medicine.

All publications, patents and patent applications cited herein are incorporated in full by reference.

### BACKGROUND

Traditionally, vaccines have been based on whole inactivated or attenuated pathogens. However, for many infectious diseases such as malaria, this approach is impractical and the focus of research has changed to the development of ‘subunit vaccines’ expressing only those pathogen-derived antigens that induce immune correlates of protection.

Subunit vaccines present an antigen to the immune system without introducing a whole infectious organism. One such method involves the administration of a specific, isolated protein from an infectious organism. However, this technique often induces only a weak immune response and the isolated proteins may have a different three-dimensional structure than the protein in its normal context, resulting in the production of antibodies that may not recognize the infectious organism.

An alternative method has therefore been developed which utilizes viral vectors for the delivery of antigens. Viruses are obligate intracellular parasites which replicate by transfecting their DNA into a host cell, and inducing the host cell to express the viral genome. This reproductive strategy has been harnessed to create vectored vaccines by creating recombinant, non-replicating viral vectors which carry one or more heterologous transgenes. Transfection or transduction of the recombinant viral genome into the host cell results in the expression of the heterologous transgene in the host cell. When the heterologous transgene encodes an antigen, for example, expression of the antigen within the host cell can elicit a protective or therapeutic immune response by the host immune system. As such, the viral vectors may function as effective vaccines. Alternatively, the heterologous transgene may encode a functional allele of a gene, expression of which can be used to counteract the effects of a deleterious mutant allele of the gene, in a process known as gene therapy.

Particularly suitable for use as viral vectors are adenoviruses. Adenoviruses are non-enveloped viruses, approximately 90-100 nm in diameter, comprising a nucleocapsid and a linear double stranded DNA genome. The viral nucleocapsid comprises penton and hexon capsomers. A unique fibre is associated with each penton base and aids in the attachment of the virus to the host cell via the Coxsackie-adenovirus receptor on the surface of the host cell. Over 50 serotype strains of adenoviruses have been identified, most of which cause respiratory tract infections, conjunctivitis and gastroentiritus in humans. Rather than integrating into the host genome, adenoviruses normally replicate as episomal elements in the nucleus of the host cell. The genome of adenoviruses comprises 4 early transcriptional units (E1, E2, E3 and E4), which have mainly regulatory functions and prepare the host cell for viral replication. The genome also comprises 5 late transcriptional units (L1, L2, L3, L4 and L5), which encode structural proteins including the penton (L2), the hexon (L3), the scaffolding protein (L4) and the fiber protein (L5), which are under the control of a single promoter. Each extremity of the genome comprises an Inverted Terminal Repeat (ITR) which is necessary for viral replication.

Recombinant adenoviruses were originally developed for gene therapy, but the strong and sustained transgene-specific immune responses elicited by these gene delivery agents prompted their use as vaccine carriers. In addition to being highly immunogenic, adenoviruses offer many other advantages for clinical vaccine development. The adenoviral genome is relatively small (between 26 and 45 kbp), well characterised and easy to manipulate. The deletion of a single transcriptional unit, E1, renders the virus replication-incompetent which increases its predictability and reduces side effects in clinical applications. Recombinant adenoviruses can accommodate relatively large transgenes, in some cases up to 8 kb, allowing flexibility in subunit design, and have a relatively broad tropism facilitating transgene delivery to a wide variety of cells and tissues. Importantly for clinical applications, methods for scaled-up production and purification of recombinant adenoviruses to high titre are well established. Thus far, subgroup C serotypes AdHu2 or AdHu5 have predominantly been used as vectors.

However, the first generation of vaccine vectors based on the archetypal human adenovirus AdHu5 showed poor efficacy in clinical trials, despite encouraging preclinical data1. It was subsequently discovered that a large proportion of human adults harbour significant titres of neutralising antibodies to common human serotypes such as AdHu2 and AdHu5, as a result of natural infection. Neutralising antibodies could reduce the potency of viral vector vaccines by blocking viral entry into host cells and hence delivery of the target transgene.

The occurrence of pre-existing anti-vector immunity is being addressed through the development of new adenoviral vectors based on serotypes to which the human population is less likely to have been exposed, including those of chimpanzee origin2,3. However, some such chimpanzee adenoviral vectors have limited efficacy on the grounds of unexplained immunity in human populations, varying levels of cross-reactivity with human adenoviruses, and sub-optimal growth in transformed cell lines. In addition, it is advantageous to have a range of different adenoviral vectors available for use in immunising against different diseases, on the grounds that induction of neutralising antibodies against a vector may prevent its re-administration for another indication.

Thus, there continues to be a need in the art for highly immunogenic, non-human adenoviral vectors which effectively deliver the target transgene, minimize the effect of pre-existing immunity to adenovirus serotypes and replicate efficiently in transformed cell lines.

### SUMMARY OF INVENTION

In a first aspect, the present invention provides the complete genomic sequence of a chimpanzee adenovirus referred to herein as AdY25.

In a second aspect, the present invention provides an adenovirus vector comprising a capsid derived from chimpanzee adenovirus AdY25, wherein said capsid encapsidates a nucleic acid molecule comprising an exogeneous nucleotide sequence of interest operably linked to expression control sequences which direct the translation, transcription and/or expression thereof in an animal cell and an adenoviral packaging signal sequence.

A third aspect provides immunogenic compositions comprising the adenoviral vector according to the second aspect, optionally in combination with one or more additional active ingredients, a pharmaceutically acceptable carrier, diluent, excipient or adjuvant.

A fourth aspect provides the use of the adenoviral vector according to the second aspect or the immunogenic composition according to the third aspect in medicine. In particular, the adenoviral vector and immunogenic compositions are provided for delivery of a transgene into a host cell, elicitation of an immune response in an animal, boosting an immune response in an animal, treating or preventing at least one disease, inducing an immune response in an animal that will break tolerance to a self antigen and gene therapy.

A fifth aspect of the present invention provides a polynucleotide sequence encoding the adenoviral vector according to the second aspect of the present invention.

A sixth aspect of the present invention provides a host cell transduced with the viral vector according to the second aspect of the present invention.

A seventh aspect of the present invention provides a method of producing the viral vector according to the second aspect of the present invention, preferably by generating a molecular clone of AdY25 in a Bacterial Artificial Chromosome (BAC).

An eighth aspect of the present invention therefore provides a Bacterial Artificial Chromosome (BAC) clone comprising the polynucleotide sequence according to the fifth aspect of the present invention.

A ninth aspect of the present invention provides a packaging cell line producing the viral vector according to the second aspect of the present invention.

A tenth aspect of the present invention provides an adenoviral vector other than AdHu5 having a nucleic acid molecule comprising the E4Orf4, E4Orf6 and E4Orf6/7 coding regions from AdHu5.

### FIGURES

The present invention is described with reference to the following figures in which:

FIGS. 1A and 1B show a phylogenetic sequence alignment of the amino acid sequences of (FIG. 1A) the hexon protein and (FIG. 1B) the fiber protein of different adenovirus serotypes. Sequences are clustered into the six adenovirus groups A-F.

FIG. 2 shows a phylogenetic sequence alignment based on the whole genomic nucleotide sequence of wild type adenoviruses of different species. Sequences are clustered into the six adenovirus groups A-F.

FIG. 3A is a histogram of the viral yield (infectious units/ml) of AdHu5 and three Ad Y25-based vectors expressing Green Fluorescent Protein (GFP): i) AdY25 E4 wildtype (“Y25E4 wt”); ii) AdY25 E4 AdHu5 Orf6 (“Y25Ad5E4Orf6”) and iii) AdY25 AdHu5 E4Orf4/6/7 (“AdChOX1”).

FIG. 3B is a histogram of the ratio of GFP foci to anti-hexon titer for AdHu5, AdCh63, AdY25 E4 wildtype and the constructs A-E as described in FIG. 3C, all expressing the TIPeGFP antigen.

FIG. 3C is a table detailing the construction of the E4-modified AdY25 vector constructs A, B, C, D and E.

FIG. 3D is a histogram of the ratio of marker gene: hexon titer for AdChOX1-based vectors expressing TIPeGFP, having either GFP or mCherry fluorescent transgenes. All data is representative of at least two independent experiments. Error bars show mean and SEM.

FIG. 4 is a graphical representation of cellular immunogenicity (spot forming cells (SFC)/million) of ChAdOX1 as compared to AdCh63 and AdCh68.

FIGS. 5A and 5B are graphical representations of the effect of E4 modification on IFN-7 spleen ELISpot responses (SFC/million) to two epitopes, Pb9 (FIG. 5A) and P15 (FIG. 5B), two weeks after intramuscular immunisation of Balb/c mice (4/group) with either 108 or 106 infectious units (ifu) of AdY25-based vectors with the following E4 regions: i) wildtype E4 region (“E4 wt”); ii) E4Orf6 from AdHu5 (“E4Orf6”); or iii) E4Orf4, 6 and 7 from AdHu5 (“E4Orf4/6/7”).

FIGS. 6A and 6B are histograms showing the prevalence of vector-neutralising antibodies in human sera from (FIG. 6A) the UK and (FIG. 6B) the Gambia, against Y25Ad5E4Orf6 (referred to in FIGS. 6A and 6B as “ChAdOX1”) and AdCh63.

FIG. 7 is a graphical representation of the humoral immunogenicity of ChAdOX1 and AdCh68-based vectors carrying TIPeGFP antigen. After 56 days post prime, mice were boosted with 106 pfu MVA-TIPeGFP Serum was collected and responses measured by endpoint ELISA a) 50 days post prime and b) 10 days post boost. Mean and significance indicated. Statistical analyses performed by one way ANOVA. Dotted line indicates limit of detection of the assay.

FIG. 8A is a graphical representation of cellular immunogenicity (spot forming cells (SFC)/million splenocyltes) of ChAdOX1 vector carrying the Mycobacterium tuberculosis Ag85A antigen, at three different doses. Cellular immune responses to Ag85A were determined by IFN-γ ELIspot assay using splenocytes stimulated with synthetic peptides corresponding to the known immunodominant CD4+ T cell H-2d restricted epitope in Ag85A (p15).

FIG. 8B is a graphical representation of cellular immunogenicity (spot forming cells (SFC)/million splenocyltes) of ChAdOX1 carrying the Mycobacterium tuberculosis Ag85A antigen, at three different doses. Cellular immune responses to Ag85A were determined by IFN-7 ELIspot assay using splenocytes stimulated with synthetic peptides corresponding to the known immunodominant CD8+ T cell H-2d restricted epitope in Ag85A (p11).

FIG. 9 is a graphical representation of cellular immunogenicity (spot forming cells (SFC)/million splenocyltes) of ChAdOX1 and HAdV-5 carrying the nucleoprotein (NP) and matrix protein 1 (M1) of Influenza A virus, at two different doses. Cellular immune responses to nucleoprotein (NP) were determined by IFN-7 ELIspot assay using splenocytes stimulated with synthetic peptides corresponding to the known immunodominant CD8+ T cell H-2d restricted epitope in NP.

### DETAILED DESCRIPTION

The present invention relates to novel adenoviral vectors derived from a chimpanzee adenovirus, AdY25, immunogenic compositions thereof and their use in medicine.

AdY25 is a chimpanzee adenovirus which has been sequenced for the first time by the present inventors. The nucleotide sequence is provided in SEQ ID NO. 1.

A first aspect of the present invention therefore provides a nucleic acid molecule having the sequence of SEQ ID NO. 1. In one embodiment, the nucleic acid molecule is isolated.

The person skilled in the art will appreciate that there are homologues, equivalents and derivatives of all of the nucleic acid sequences described herein. Thus, the invention also encompasses nucleic acid molecules having a sequence substantially identical to the nucleic acid sequences described herein over their entire length.

One of skill in the art will appreciate that the present invention can also include variants of those particular nucleic acid molecules which are exemplified herein. These may occur in nature, for example because of strain variation. For example, additions, substitutions and/or deletions are included. One of skill in the art will also appreciate that variation from the particular nucleic acid molecules exemplified herein will be possible in view of the degeneracy of the genetic code. Preferably, the variants have substantial identity to the nucleic acid sequences described herein over their entire length.

As used herein, nucleic acid sequences which have “substantial identity” preferably have at least 80%, 90%, 91%, 92%, 93%, 94%, 95% 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4% 99.5%, 99.6%, 99.7%, 99.8% or 99.9% identity with said sequences. Desirably, the term “substantial identity” indicates that said sequence has a greater degree of identity with any of the sequences described herein than with prior art nucleic acid sequences.

When comparing nucleic acid sequences for the purposes of determining the degree of homology or identity one can use programs such as BESTFIT and GAP (both from the Wisconsin Genetics Computer Group (GCG) software package). BESTFIT, for example, compares two sequences and produces an optimal alignment of the most similar segments. GAP enables sequences to be aligned along their whole length and finds the optimal alignment by inserting spaces in either sequence as appropriate. Suitably, in the context of the present invention, when discussing identity of nucleic acid sequences, the comparison is made by alignment of the sequences along their whole length. The above applied mutatis mutandis to all nucleic acid sequences disclosed in the present application.

Preferably, the nucleic acid molecule according to the first aspect has a sequence at least 98% identical to SEQ ID NO.1, more preferably at least 98.6% identical to SEQ ID NO.1.

Preferably, the nucleic acid molecule according to the first aspect comprises one or more nucleotide sequences selected from the group consisting of;
(a) nucleotides 18302 to 21130 of SEQ ID NO. 1 or a sequence substantially identical thereto;
(b) nucleotides 13891 to 15486 of SEQ ID NO. 1 or a sequence substantially identical thereto; and
(c) nucleotides 32290 to 33621 of SEQ ID NO. 1 or a sequence substantially identical thereto.

These nucleotide sequences encode the (a) hexon, (b) penton and (c) fibre capsid proteins of AdY25, the exterior regions of which determine the properties of the viral vector, including serotype.

The nucleic acid molecule according to the first aspect may also comprise one or more nucleotide sequences selected from the group consisting of:
(a) a nucleotide sequence encoding a hexon protein comprising the amino acid sequence of SEQ ID NO.2, or a sequence at least 98.2% identical thereto; or a nucleotide sequence encoding a hexon protein having a sequence at least 98.2% identical to the protein encoded by nucleotides 18302 to 21 130 of SEQ ID NO. 1;
(b) a nucleotide sequence encoding a penton protein comprising the amino acid sequence of SEQ ID NO.3, or a sequence at least 98.3% identical thereto; or a nucleotide sequence encoding a penton protein having a sequence at least 98.3% identical to the protein encoded by nucleotides 13891 to 15486 of SEQ ID NO. 1; and
(c) a nucleotide sequence encoding a fiber protein comprising the amino acid sequence of SEQ ID NO.4 or a sequence at least 99.1% identical thereto; or a nucleotide sequence encoding a fiber protein having a sequence at least 99.1% identical to the protein encoded by nucleotides 32290 to 33621 of SEQ ID NO. 1.

Nucleic acid molecules comprising a sequence complementary to the nucleic acid molecule according to the first aspect of the present invention are within the scope of the present invention.

Nucleic acid molecules which hybridize only to the nucleic acid molecule according to the first aspect of the present invention are also encompassed by the present application. Thus, the conditions used for hybridisation are sufficiently stringent that only such nucleic acid sequences would remain hybridised. The person skilled in the art would easily be able to determine such conditions.

The nucleic acid can be DNA, including cDNA, RNA including mRNA or PNA (peptide nucleic acid) or a mixture thereof.

Table 1 provides an overview of the wildtype AdY25 sequences disclosed herein:

| SEQ | | |
|---|---|---|
| ID | | Corresponding nucleotides in |
| NO. | Description | SEQ ID NO. 1 |
| 1 | Genome | N/A |
| | (nucleotide sequence) | |
| 2 | Hexon protein | Nucleotides 18302 to 21130 (L3) |
| 3 | Penton protein | Nucleotides 13891 to 15486 (L2) |
| 4 | Fibre protein | Nucleotides 32290 to 33621 (L5) |
| 5 | E1A | Nucleotides 577 to 1143 and 1237 |
| | | to 1443 |
| 6 | EIB 19 KDa | Nucleotides 1602 to 2165 |
| 7 | E1B 55 KDa | Nucleotides 1907 to 3406 |
| 8 | pIX | Nucleotides 3491 to 3919 |
| 9 | IVa2 | Nucleotides 5587 to 5602 and 3978 |
| | | to 5311 (E2) |
| 10 | Polymerase | Nucleotides 13838 to 13846 and |
| | | 5081 to 8662 (E2) |
| 11 | pTP | Nucleotides 13838 to 13846 and |
| | | 8463 to 10392 (E2) |
| 12 | 52/55 kDa | Nucleotides 10827 to 12017 (L1) |
| 13 | IIIa | Nucleotides 12041 to 13807 (L1) |
| 14 | VII | Nucleotides 15493 to 16074 |
| 15 | V | Nucleotides 16119 to 17141 |
| 16 | Mu | Nucleotides 17161 to 17394 |
| 17 | VI | Nucleotides 17470 to 18201 |
| 18 | Endoprotease | Nucleotides 21146 to 21775 |
| 19 | DNA binding | Nucleotides 21852 to 23390 |
| | protein | |
| 20 | 100 kDa | Nucleotides 23419 to 25827 (L4) |
| 21 | 22 KDa | Nucleotides 25544 to 26098 |
| 22 | 33 KDa | Nucleotides 25544 to 25871 and |
| | | 26041 to 26372 (L4) |
| 23 | pVIII | Nucleotides 25602 to 26285 (L4) |
| 24 | E3 12.5 KDa | Nucleotides 27139 to 27459 |
| 25 | E3 CRIaI | Nucleotides 27413 to 28051 |
| 26 | E3 gp19 KDa | Nucleotides 28033 to 28563 |
| 27 | E3 22.3 KDa | Nucleotides 29350 to 29979 |
| 28 | E3 31 KDa | Nucleotides 29999 to 30907 |
| 29 | E3 10.4 KDa | Nucleotides 30916 to 31191 |
| 30 | E3 15.2 KDa | Nucleotides 31200 to 31643 |
| 31 | E3 14.7 KDa | Nucleotides 31636 to 32040 |
| 32 | E4 Orf 6/7 | Nucleotides 34688 to 34861 and |
| | | 33716 to 33965 |
| 33 | E4 Orf 6 | Nucleotides 33965 to 34861 |
| 34 | E4 Orf 4 | Nucleotides 34764 to 35132 |
| 35 | E4 Orf 3 | Nucleotides 35141 to 35494 |
| 36 | E4 Orf 2 | Nucleotides 35491 to 35880 |
| 37 | E4 Orf 1 | Nucleotides 35930 to 36304 |

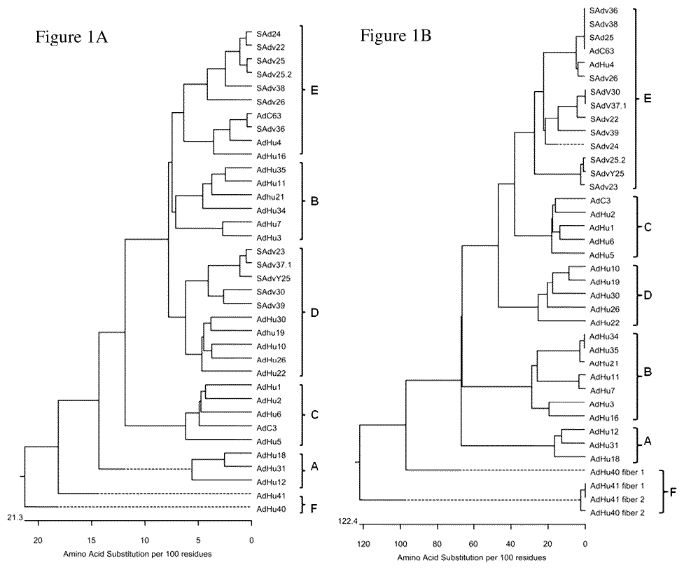

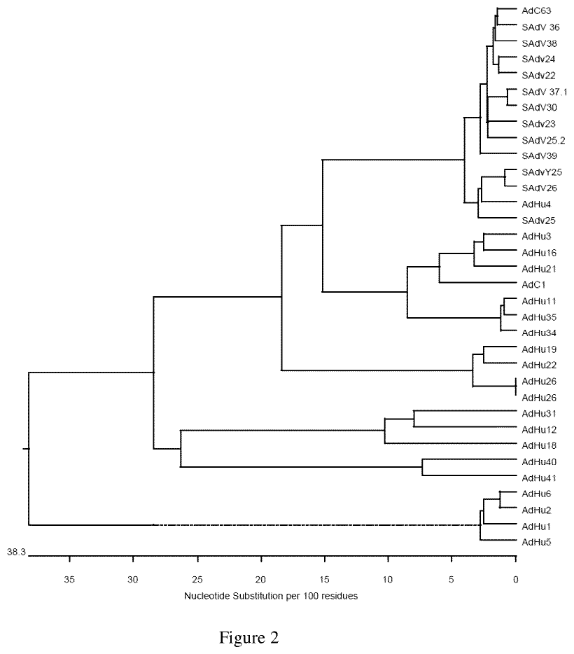

The genome sequence data has confirmed early serological studies that simian AdY25 is closely related to human group E adenovirus, AdHu44. Alignment of the amino acid sequences of hexon and fibre proteins from different adenoviral serotypes have been used to create the phylogenetic trees in FIGS. 1A and 1B. These are the major surface-exposed capsid components and are believed to be the primary determinants of vector tropism. Alignment of whole genomic nucleotide sequences of different adenoviral species have been used to create the phylogenetic tree in FIG. 2. The genome and the fibre proteins align AdY25 with the group E adenoviruses. However, the hexon proteins align AdY25 with the group D adenoviruses.

Merely for the convenience of those of skill in the art, a sample of E. coli strain DH10B containing bacterial artificial chromosomes (BACs) containing the cloned genome of chimpanzee adenovirus Y25 (pBACe3.6 Y25, cell line name “Y25”) was deposited by Isis Innovation Limited on 24 May 2012 with the European Collection of Cell Cultures (ECACC) at the Health Protection Agency Culture Collections, Health Protection Agency, Porton Down, Salisbury SP4 0JG, United Kingdom under the Budapest Treaty and designated by provisional accession no. 12052401.

The E. coli containing the BAC is a class I genetically modified organism. The genotype of E. coli strain DH10B is: F-mcrA A(mrr-hsdRMS-mcrBC)

Φ80dlacZΔM15 ΔlacX74 endA1 recA1 deoR Δ(ara,leu)7697 araD139 galU galK nupG rpsL λ−. Chimpanzee adenovirus Y25 is provisionally classified within the species Human adenovirus E based on the nucleotide sequence of the viral DNA polymerase.

The BAC propagates within the bacteria during replication and can be maintained by selection with chloramphenicol. The E. coli strain DH10B containing the BAC into which the genome is cloned can be propagated in Luria-Bertani broth or agar containing 12.5 g/mL chloramphenicol at 37° C.

Converting the BAC clones of the viral genomes into viruses (“rescue”) can be carried out by the following steps. The E. coli host is propagated and the BAC DNA is purified from the bacteria according to standard methods. The DNA is linearised with the restriction endonuclease PmeI and transfected into any cell line supporting growth of human adenoviruses (e.g. A549 cells). The resulting adenovirus can then be propagated and purified for use as a vaccine, for example. All of these reagents and cells are publicly available. If the deposition were rescued, the resulting virus would be a wild-type adenovirus.

In respect of all designated states to which such action is possible and to the extent that it is legally permissible under the law of the designated state, it is requested that a sample of the deposited material be made available only by the issue thereof to an independent expert, in accordance with the relevant patent legislation, e.g. Rule 32(1) EPC, Rule 13(1) and Schedule 1 of the UK Patent Rules 2007, Regulation 3.25(3) of the Australian Patent Regulations and generally similar provisions mutatis mutandis for any other designated state.

Furthermore, merely for the convenience of those of skill in the art, a sample of E. coli strain DH10B containing bacterial artificial chromosomes (BACs) containing the cloned genome of chimpanzee adenovirus Y25 with deletion of the E1 region (pBACe3.6 Y25delE1, cell line name “Y25delE1”) was deposited by Isis Innovation Limited on 24 May 2012 with the European Collection of Cell Cultures (ECACC) at the Health Protection Agency Culture Collections, Health Protection Agency, Porton Down, Salisbury SP4 0JG, United Kingdom under the Budapest Treaty and designated by provisional accession no. 12052402.

The E. coli containing the BAC is a class I genetically modified organism. The genotype of E. coli strain DH10B is: F-mcrA A(mrr-hsdRMS-mcrBC)

Φ80dlacZΔM15 ΔlacX74 endA1 recA1 deoR Δ(ara,leu) 7697 araD139 gal U galK nupG rpsL λ−. Chimpanzee adenovirus Y25 is provisionally classified within the species Human adenovirus E based on the nucleotide sequence of the viral DNA polymerase.

The BAC propagates within the bacteria during replication and can be maintained by selection with chloramphenicol. The E. coli strain DH10B containing the bacterial artificial chromosomes into which the genomes are cloned can be propagated in Luria-Bertani broth or agar containing 12.5 μg/mL chloramphenicol at 37° C.

Converting the BAC clones of the viral genomes into viruses (“rescue”) can be carried out by the following steps. The E. coli host is propagated and the BAC DNA is purified from the bacteria according to standard methods. The DNA is linearised with the restriction endonuclease PmeI and transfected into HEK293 cells (or a similar E1 complementing cell line). The resulting adenovirus can then be propagated and purified for use as a vaccine for example. All of these reagents and cells are publicly available. If the deposition were rescued, the resulting virus would be a class I genetically modified organism.

In respect of all designated states to which such action is possible and to the extent that it is legally permissible under the law of the designated state, it is requested that a sample of the deposited material be made available only by the issue thereof to an independent expert, in accordance with the relevant patent legislation, e.g. Rule 32(1) EPC, Rule 13(1) and Schedule 1 of the UK Patent Rules 2007, Regulation 3.25(3) of the Australian Patent Regulations and generally similar provisions mutatis mutandis for any other designated state.

A specific embodiment of the first aspect of the present invention provides the complete genomic sequence of a chimpanzee adenovirus referred to herein as AdY25, wherein said genomic sequence comprises or consists of the genomic sequence deposited in a BAC in E. coli strain DH10B by Isis Innovation Limited on 24 May 2012 with the European Collection of Cell Cultures (ECACC) at the Health Protection Agency Culture Collections, Health Protection Agency, Porton Down, Salisbury SP4 0JG, United Kingdom under the Budapest Treaty and designated by provisional accession no. 12052401, or the genomic sequence deposited in a BAC in E. coli strain DH10B by Isis Innovation Limited on 24 May 2012 with the European Collection of Cell Cultures (ECACC) at the Health Protection Agency Culture Collections, Health Protection Agency, Porton Down, Salisbury SP4 0JG, United Kingdom under the Budapest Treaty and designated by provisional accession no. 12052402.

The inventors have discovered that viral vectors based on the newly sequenced AdY25 can be highly effective. A second aspect of the present invention therefore provides an adenovirus vector comprising a capsid derived from chimpanzee adenovirus AdY25, wherein said capsid encapsidates a nucleic acid molecule comprising an exogeneous nucleotide sequence of interest operably linked to expression control sequences which direct the translation, transcription and/or expression thereof in an animal cell and an adenoviral packaging signal sequence.

As used herein, the phrase “viral vector” refers to a recombinant virus or a derivative thereof which is capable of introducing genetic material, including recombinant DNA, into a host cell or host organism by means of transduction or non-productive infection. For example, the vector of the present invention may be a gene delivery vector, a vaccine vector, an antisense delivery vector or a gene therapy vector.

As used herein, “AdY25” and “Y25” refer to the chimpanzee adenovirus AdY25 or vectors derived therefrom or based thereon. Shorthand terms are used to indicate modifications made to the wildtype virus. For example, “ΔE1” or “delE1” indicates deletion or functional deletion of the E1 locus. The phrase “Ad5E4Orf6” indicates that the viral vector comprises heterologous E4 open reading frame 6 from the Ad5 virus.

The vector of the present invention comprises a capsid derived from chimpanzee adenovirus AdY25. Preferably, the capsid comprises the native or wildtype AdY25 capsid proteins, including penton proteins, hexon proteins, fiber proteins and/or scaffolding proteins. However, one of skill in the art will readily appreciate that small modifications can be made to the capsid proteins without adversely altering vector tropism. In a particularly preferred embodiment, the vector capsid comprises one or more capsid proteins selected from the group consisting of:
(a) a hexon protein comprising the amino acid sequence of SEQ ID NO. 2 or a sequence substantially identical thereto;
(b) a penton protein comprising amino acid sequence of SEQ ID NO. 3 or a sequence substantially identical thereto; and
(c) a fibre protein comprising the amino acid sequence of SEQ ID NO. 4 or a sequence substantially identical thereto.

One of skill in the art will appreciate that the present invention can include variants of those particular amino acid sequences which are exemplified herein. Particularly preferred are variants having an amino acid sequence similar to that of the parent protein, in which one or more amino acid residues are substituted, deleted or added in any combination. Especially preferred are silent substitutions, additions and deletions, which do not alter the properties and activities of the protein of the present invention. Various amino acids have similar properties, and one or more such amino acids of a substance can often be substituted by one or more other such amino acids without eliminating a desired activity of that substance. Thus, the amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur containing side chains). Variants include naturally occurring and artificial variants. Artificial variants may be generated using mutagenesis techniques, including those applied to nucleic acid molecules, cells or organisms. Preferably, the variants have substantial identity to the amino acid sequences exemplified herein.

As used herein, amino acid sequences which have “substantial identity” preferably have at least 80%, 90%, 91%, 92%, 93%, 94%, 95% 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8% or 99.9% identity with said sequences. Desirably, the term “substantial identity” indicates that said sequence has a greater degree of identity with any of the sequences described herein than with prior art amino acid sequences.

One can use a program such as the CLUSTAL program to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. The above applied mutatis mutandis to all amino acid sequences disclosed in the present application.

Preferably, the hexon protein comprises an amino acid sequence at least 98.2% identical to SEQ ID NO. 2. Preferably, the penton protein comprises an amino acid sequence at least 98.3% identical to SEQ ID NO. 3. Preferably, the fiber protein comprises an amino acid sequence at least 99.1% identical to SEQ ID NO. 4.

The nucleotide sequences for the AdY25 hexon, penton and fibre proteins are set out in nucleotides 18302 to 21130 of SEQ ID NO.1 (hexon protein), nucleotides 13891 to 15486 of SEQ ID NO. 1 (penton protein) and nucleotides 32290-33621 of SEQ ID NO.1 (fibre protein). The vector capsid may comprise one or more AdY25 capsid proteins encoded by these nucleotide sequences or sequences substantially identical thereto.

The vector according to the second aspect of the present invention may comprise one of the hexon, penton and fibre proteins as described above, any combination of two of said proteins, or all three of said proteins.

The vector of the present invention also comprises a nucleic acid molecule. As a minimum, the nucleic acid molecule comprises an exogeneous nucleotide sequence of interest, operably linked to expression control sequences which direct the translation, transcription and/or expression thereof in an animal cell and an adenoviral packaging signal sequence.

Preferably, the exogeneous nucleotide sequence encodes a molecule of interest. The molecule of interest may be a protein, polypeptide or nucleic acid molecule of interest. The exogeneous nucleotide sequence may encode one or more, two or more or three or more molecules of interest.

Proteins and polypeptides of interest include antigens, molecular adjuvants, immunostimulatory proteins and recombinases.

Preferably, the protein or polypeptide of interest is an antigen. In one embodiment, the antigen is a pathogen-derived antigen. Preferably, the pathogen is selected from the group consisting of bacteria, viruses, prions, fungi, protists and helminthes.

Preferably, the antigen is derived from the group consisting of M. tuberculosis, Plasomodium sp, influenza virus, HIV, Hepatitis C virus, Cytomegalovirus, Human papilloma virus, malaria parasites, leishmania parasites or any mycobacterial species. Preferred antigens include TRAP, MSP-1, AMA-1 and CSP from Plasmodium, influenza virus antigens and ESAT6, TB10.4 85 A and 85B antigens from Mycobacterium tuberculosis. Particularly preferred antigens include Ag85A from Mycobacterium tuberculosis and nucleoprotein (NP) and matrix protein 1 (M1) from influenza A virus, preferably influenza A virus.

In an alternative embodiment, the antigen is a self-antigen. Suitable self-antigens include antigens expressed by tumour cells which allow the immune system to differentiate between tumour cells and other cell types. Suitable self-antigens include antigens that are either inappropriate for the cell type and/or its environment, or are only normally present during the organisms' development (e.g. foetal antigens). For example, GD2 is normally only expressed at a significant level on the outer surface membranes of neuronal cells, where its exposure to the immune system is limited by the blood-brain barrier. However, GD2 is expressed on the surfaces of a wide range of tumour cells including small-cell lung cancer, neuroblastoma, melanomas and osteosarcomas. Other suitable self-antigens include cell-surface receptors that are found on tumour cells but are rare or absent on the surface of healthy cells. Such receptors may be responsible for activating cellular signalling pathways that result in the unregulated growth and division of the tumour cell. For example, ErbB2 is produced at abnormally high levels on the surface of breast cancer tumour cells. Preferably, the self antigen comprises a tumour-associated antigen (TAA).

As used herein, the term ‘antigen’ encompasses one or more epitopes from an antigen and includes the parent antigen, and fragments and variants thereof. These fragments and variants retain essentially the same biological activity or function as the parent antigen. Preferably, they retain or improve upon the antigenicity and/or immunogenicity of the parent antigen. Generally, “antigenic” is taken to mean that the protein or polypeptide is capable of being used to raise antibodies or T cells or indeed is capable of inducing an antibody or T cell response in a subject. “Immunogenic” is taken to mean that the protein or polypeptide is capable of eliciting a potent and preferably a protective immune response in a subject. Thus, in the latter case, the protein or polypeptide may be capable of generating an antibody response and a non-antibody based immune response.

Preferably, fragments of the antigens comprise at least n consecutive amino acids from the sequence of the parent antigen, wherein n is preferably at least, or more than, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 57, 58, 59, 60, 70, 80, 90 or 100. The fragments preferably include one or more epitopic regions from the parent antigen. Indeed, the fragment may comprise or consist of an epitope from the parent antigen. Alternatively, the fragment may be sufficiently similar to such regions to retain their antigenic/immunogenic properties.

The antigens of the present invention include variants such as derivatives, analogues, homologues or functional equivalents of the parent antigen. Particularly preferred are derivatives, analogues, homologues or functional equivalents having an amino acid sequence similar to that of the parent antigen, in which one or more amino acid residues are substituted, deleted or added in any combination. Preferably, these variants retain an antigenic determinant or epitope in common with the parent antigen.

Preferably, the derivatives, analogues, homologues, and functional equivalents have an amino acid sequence substantially identical to amino acid sequence of the parent antigen.

The exogeneous nucleotide sequence may encode more than one antigen. The viral vector may be designed to express the one or more antigen genes as an epitope string. Preferably, the epitopes in a string of multiple epitopes are linked together without intervening sequences such that unnecessary nucleic acid and/or amino acid material is avoided. The creation of the epitope string is preferably achieved using a recombinant DNA construct that encodes the amino acid sequence of the epitope string, with the DNA encoding the one or more epitopes in the same reading frame. An exemplary antigen, TIPeGFP, comprises an epitope string which includes the following epitopes: E6FP, SIV-gag, PyCD4 and Py3. Alternatively, the antigens may be expressed as separate polypeptides.

One or more of the antigens or antigen genes may be truncated at the C-terminus and/or the N-terminus. This may facilitate cloning and construction of the vectored vaccine and/or enhance the immunogenicity or antigenicity of the antigen. Methods for truncation will be known to those of skill in the art. For example, various well-known techniques of genetic engineering can be used to selectively delete the encoding nucleic acid sequence at either end of the antigen gene, and then insert the desired coding sequence into the viral vector. For example, truncations of the candidate protein are created using 3′ and/or 5′ exonuclease strategies selectively to erode the 3′ and/or 5′ ends of the encoding nucleic acid, respectively. Preferably, the wild type gene sequence is truncated such that the expressed antigen is truncated by 1, 2, 3, 4, 5, 6, 7, 8, 9, 10, 11, 12, 13, 14, 15, 16, 17, 18, 19, 20 or more amino acids relative to the parent antigen. Preferably, the antigen gene is truncated by 10-20 amino acids at the C-terminus relative to the wild type antigen. More preferably, the antigen gene is truncated by 13-18 amino acids, most preferably by 15 amino acids at the C-terminus relative to the wild type antigen. Preferably, the Ag85A antigen is C-terminally truncated in this manner.

One or more of the antigen genes may also comprise a leader sequence. The leader sequence may affect processing of the primary transcript to mRNA, translation efficiency, mRNA stability, and may enhance expression and/or immunogenicity of the antigen. Preferably, the leader sequence is tissue plasminogen activator (tPA). Preferably, the tPA leader sequence is positioned N-terminal to the one or more antigens.

The leader sequence such as the tPA leaders sequence may be linked to the sequence of the antigen via a peptide linker. Peptide linkers are generally from 2 to about 50 amino acids in length, and can have any sequence, provided that it does not form a secondary structure that would interfere with domain folding of the fusion protein.

One or more of the antigen genes may comprise a marker such as the Green Fluorescent Protein (GFP) marker to facilitate detection of the expressed product of the inserted gene sequence.

One or more of the antigen genes may comprise a nucleic acid sequence encoding a tag polypeptide that is covalently linked to the antigen upon translation. Preferably the tag polypeptide is selected from the group consisting of a PK tag, a FLAG tag, a MYC tag, a polyhistidine tag or any tag that can be detected by a monoclonal antibody. The nucleic acid sequence encoding the tag polypeptide may be positioned such that, following translation, the tag is located at the C-terminus or the N-terminus of the expressed antigen or may be internal to the expressed antigen. Preferably, the tag is located at the C-terminus of the expressed antigen. In a preferred embodiment, one or more of the antigen genes encode a PK tag. A tag of this type may facilitate detection of antigen expression and clones expressing the antigen, and/or enhance the immunogenicity or antigenicity of the antigen.

If a tag polypeptide is used, nucleotides encoding a linker sequence are preferably inserted between the nucleic acid encoding the tag polypeptide and the nucleic acid encoding the expressed antigen. An exemplary linker is IPNPLLGLD (SEQ ID NO.49).

In an alternative embodiment, the exogeneous sequence of interest may be non-protein encoding. For example, the exogeneous nucleotide sequence may be an miRNA or immunostimulatory RNA sequence.

The adenoviral vector may comprise one or more exogeneous nucleotide sequences, for example 1, 2 or 3 or more exogeneous nucleotide sequences. Preferably, each exogeneous nucleotide sequence embodies a transgene. The exogeneous nucleotide sequence embodying the transgene can be a gene or a functional part of the gene. The adenoviral vector may comprise one nucleotide sequence encoding a single molecule of interest. Alternatively, the adenoviral vector may comprise one nucleotide sequence or more than one nucleotide sequence encoding more than one molecule of interest.

Preferably, the exogeneous nucleotide sequence is located in a nucleic acid molecule that contains other, adenoviral sequences. The exogeneous nucleotide sequence may be inserted into the site of a partially or fully deleted AdY25 gene, for example into the site of an E1 deletion or an E3 deletion. The exogeneous nucleotide sequence may be inserted into an existing AdY25 gene region to disrupt the function of that region. Alternatively, the exogeneous nucleotide sequence may be inserted into a region of the AdY25 genome with no alteration to the function or sequence of the surrounding genes.

The exogeneous nucleotide sequence or transgene is preferably operably linked to regulatory sequences necessary to drive translation, transcription and/or expression of the exogeneous nucleotide sequence/transgene in a host cell, for example a mammalian cell. As used herein, the phrase “operably linked” means that the regulatory sequences are contiguous with the nucleic acid sequences they regulate or that said regulatory sequences act in trans, or at a distance, to control the regulated nucleic acid sequence. Such regulatory sequences include appropriate expression control sequences such as transcription initiation, termination, enhancer and promoter sequences, efficient RNA processing signals, such as splicing and polyadenylation signals, sequences that enhance translation efficiency and protein stability and sequences promote protein secretion. Additionally they may contain sequences for repression of transgene expression, for example during production in cell lines expression a transactivating receptor. Promoters and other regulatory sequences which control expression of a nucleic acid have been identified and are known in the art. Preferably, the promoter is selected from the group consisting of human CMV promoters, simian CMV promoters, murine CMV promoters, ubiquitin, the EF1 promoter, frog EF1 promoter, actin and other mammalian promoters. Most preferred are human CMV promoters and in particular the human CMV major immediate early promoter.

The exogeneous nucleotide sequence(s) of interest may be introduced into the viral vector as part of a cassette. As used herein, the term “cassette” refers to a nucleic acid molecule comprising at least one nucleotide sequence to be expressed, along with its transcriptional and translational control sequences to allow the expression of the nucleotide sequence(s) in a host cell, and optionally restriction sites at the 5′ and 3′ ends of the cassette. Because of the restriction endonuclease sites, the cassettes can easily be inserted, removed or replaced with another cassette. Changing the cassette will result in the expression of different sequence(s) by the vector into which the cassette is incorporated. Alternatively, any method known to one of skill in the art could be used to construct, modify or derive said cassette, for example PCR mutagenesis, In-Fusion®, recombineering, Gateway® cloning, site-specific recombination or topoisomerase cloning.

The expression control sequences preferably include the adenovirus elements necessary for replication and virion encapsidation. Preferably, the elements flank the exogeneous nucleotide sequence. Preferably, the Y25 vector comprises the 5′ inverted terminal repeat (ITR) sequences of Y25, which function as origins of replication, and 3′ ITR sequences.

The packaging signal sequence functions to direct the assembly of the viral vector.

As one of skill in the art will appreciate, there are minimum and maximum constraints upon the length of the nucleic acid molecule that can be encapsidated in the viral vector. Therefore, if required, the nucleic acid molecule may also comprise “stuffing”, i.e. extra nucleotide sequence to bring the final vector genome up to the required size. Preferably, the nucleic acid molecule comprises sufficient “stuffing” to ensure that the nucleic acid molecule is about 80% to about 108% of the length of the wild-type nucleic acid molecule.

The nucleic acid molecule may also comprise one or more genes or loci from the AdY25 genome. The wildtype AdY25 genome comprises 4 early transcriptional units (E1, E2, E3 and E4), which have mainly regulatory functions and prepare the host cell for viral replication. The genome also comprises 5 late transcriptional units (L1, L2, L3, L4 and L5), which encode structural proteins including the penton (L2), the hexon (L3), the scaffolding protein (L4) and the fiber protein (L5), which are under the control of a single promoter. Each extremity of the genome comprises an Inverted Terminal Repeat (ITR) which is necessary for viral replication. The viral vector of the present invention may comprise the complete native AdY25 genome, into which the exogeneous nucleotide sequence has been inserted. However, one of skill in the art will appreciate that various modifications to the native AdY25 genome are possible, and indeed desirable, when creating a viral vector.

One or more native AdY25 genes may be deleted, functionally deleted or modified to optimise the viral vector. As used herein, the phrase “deleted” refers to total deletion of a gene, whilst “functional deletion” refers to a partial deletion of a gene/locus, or some other modification such as a frame shift mutation, which destroys the ability of the adenovirus to express the gene/locus or renders the gene product non-functional. The AdY25 genome may be modified to increase the insert capacity or hinder replication in host cells and/or increase growth and yield of the viral vector in transformed packaging cell lines. One of skill in the art will appreciate that any number of early or late genes can be functionally deleted. Replication of such modified viral vectors will still be possible in transformed cell lines which comprise a complement of the deleted genes. For example, the viral proteins necessary for replication and assembly can be provided in trans by engineered packaging cell lines or by a helper virus.

Therefore, in addition to the exogeneous nucleotide sequence, the vector of the present invention may comprise the minimal adenoviral sequences, the adenoviral genome with one or more deletions or functional deletions of particular genes, or the complete native adenoviral genome, into which has been inserted the exogeneous nucleotide sequence.

Preferably, the vector of the present invention comprises the native Y25 late transcriptional units (L1-L5) and/or the native Y25 Inverted Terminal Repeats (ITR) or sequences substantially identical thereto. The amino acid sequences of the native L1, L2, L3, L4, L5 loci, and the corresponding nucleic sequences, are set out in Table 1, above.

Preferably, one or more of the early transcriptional units are modified, deleted or functionally deleted.

In one embodiment, the viral vector is non-replicating or replication-impaired. As used herein, the term “non-replicating” or “replication-impaired” means not capable of replicating to any significant extent in the majority of normal mammalian cells, preferably normal human cells. It is preferred that the viral vector is incapable of causing a productive infection or disease in the human patient. However, the viral vector is preferably capable of stimulating an immune response. Viruses which are non-replicating or replication-impaired may have become so naturally, i.e. they may be isolated as such from nature. Alternatively, the viruses may be rendered non-replicating or replication-impaired artificially, e.g. by breeding in vitro or by genetic manipulation. For example, a gene which is critical for replication may be functionally deleted. Preferably, the adenoviral vector replication is rendered incompetent by functional deletion of a single transcriptional unit which is essential for viral replication. Preferably, the E1 gene/locus is deleted or functionally deleted. The E1 gene/locus may be replaced with a heterologous transgene, for example a nucleotide sequence or expression cassette encoding a protein or polypeptide of interest.

The wildtype AdY25 E1 amino acid sequence, and the corresponding nucleic acid sequence, are set out in Table 1, above.

As discussed herein, the recombinant adenovirus may be created by generating a molecular clone of AdY25 in a Bacterial Artificial Chromosome (BAC), and the E1 locus is preferably deleted by including an extra homology flank downstream of the adenovirus E1 region to enable simultaneous deletion of E1 during homologous recombination between the AdY25 viral DNA and a linearised BAC “rescue vector”, as described in Example 1.

Preferably, the viral vector according to the present invention comprises one or more recombination sites to enable the insertion of one or more transgenes or cassettes comprising the exogeneous nucleotide sequence. Preferably, the recombination sites comprise phage lambda site specific recombination sites. These recombination sites may be introduced at any suitable locus, but are preferably introduced at the Ad E1 locus. Thus, the non-replicating or replication-impaired vector may be prepared by replacing the E1 gene with a nucleotide sequence encoding the protein or polypeptide of interest. Preferably, the recombination sites attR1 and attR2 are introduced at the Ad E1 locus as part of an Invitrogen Gateway® destination cassette as described in Example 1.

Preferably, the vector lacks an adenovirus E3 gene/locus. Deletion of the adenovirus E3 region increases the insert capacity of the new vector by approximately 5 kb. Deletion of E3 has little consequence to viral vector yield since this region is not required for virus replication and therefore does not need to be provided in trans in the packaging cell line. The E3 locus may be deleted using GalK recombineering as described in Example 2.

The wildtype AdY25 E3 amino acid sequence, and the corresponding nucleic acid sequence, are set out in Table 1, above.

In a particularly preferred embodiment of the present invention, both the E1 and E3 loci are deleted from the AdY25 genome.

Preferably, the vector of the present invention comprises the native E2 locus. E2 is a transcriptional unit comprising the open reading frames encoding the Polymerase, PTP and IVa2 proteins. The wildtype AdY25 E4 amino acid sequence, and the corresponding nucleotide sequence, are set out in Table 1, above. Preferably, the vector of the present invention comprises a nucleotide sequence encoding E2 or a sequence substantially identical thereto.

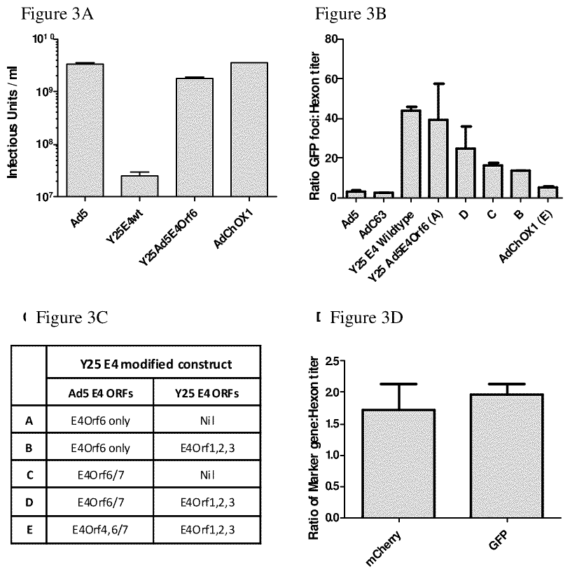

As stated above, the viral vectors of the present invention may be produced in engineered cell lines containing a complement of any deleted genes required for viral replication. However, replication of viral vectors according to the present invention may be sub-optimal in cells designed to facilitate replication of other serotypes. For example, as shown in FIG. 3A, the first generation of AdY25-based vectors comprising the wildtype E4 locus were found to grow inefficiently in HEK293 cells and yield was approximately two logs lower than for comparable AdHu5-based vectors. It is hypothesized that the low yield resulted from suboptimal interaction between the cell-expressed E1 proteins (designed to support propagation of AdHu5 viruses) and vector-encoded E4 gene products. Therefore, the adenoviral vectors according to the present invention preferably further comprise one or more modifications designed to optimise vector growth and yield in transformed cell lines, such as HEK293, expressing the genes functionally deleted in the adenoviral vector according to the present invention.

In one embodiment, the native E4 region may be replaced in its entirety with a heterologous E4 region from other serotype(s), which heterologous E4 region preferably increases vector yield and growth in a transformed cell line. For example, the native E4 region may be replaced with the E4 region from AdHu5 to increase vector yield and growth in HEK293.

The adenovirus E4 region comprises at least 6 Open Reading Frames (ORFs or Orfs). Thus, in an alternative embodiment, one or more of the ORFs in the E4 region may be replaced with one or more heterologous ORFs from the E4 region of other adenoviral serotype(s), which heterologous ORF(s) preferably increase(s) vector yield and growth in a transformed cell line. Preferably, 1, 2, 3, 4, 5 or 6 ORFs in the E4 region may be replaced 1, 2, 3, 4, 5 or 6 heterologous ORFs from the E4 region of other serotype(s), e.g. AdHu5.

Of particular importance for viral replication in HEK293 cells is the gene product of E4Orf6, a multifunctional protein implicated in late viral mRNA splicing and selective export of viral mRNA, viral DNA synthesis and inhibition of apoptosis. Suboptimal interaction between E4Orf6 and the cell-expressed E1B-55K is believed to reduce the yield of AdChOX1 vectors in HEK293 cells. Therefore, the native E4Orf6 region may be replaced with a heterologous E4Orf6 region. For example, the entire native E4 locus may be replaced with the E4Orf6 gene from AdHu5, as described in Example 3. The amino acid sequence of E4Orf6 from AdHu5 is found in SEQ ID NO.40. A corresponding nucleotide sequence is found at nucleotides 28248 to 29132 of SEQ ID NO. 38. In one embodiment, the vector of the present invention comprises the nucleotide sequence of AdHu5E4Orf6 or a sequence substantially identical thereto. As described in Example 3 and shown in FIG. 3A, this modification was found to improve viral yield and growth.

In a preferred embodiment, more than one ORF in the E4 region is replaced with more than one heterologous ORF from the E4 region of other serotype(s). For example, native E4Orf4, E4Orf6 and E4Orf7 may be replaced with the E4Orf4, E4Orf6 and E4Orf6/7 coding regions from AdHu5. In a particularly preferred embodiment, the recombinant E4 region comprises the E4Orf1, E4Orf2 and E4Orf3 coding regions from AdY25 and the E4Orf4, E4Orf6 and E4Orf6/7 coding regions from AdHu5. The amino acid sequence of E4Orf4 from AdHu5 is found in SEQ ID NO. 41. A corresponding nucleotide sequence is found at nucleotides 29053 to 29397 of SEQ ID NO. 38. The amino acid sequence of the E4Orf6 from AdHu5 is found in SEQ ID NO. 40. A corresponding nucleotide sequence is found at nucleotides 28248 to 29132 of SEQ ID NO. 38. The amino acid sequence of the E4Orf6/7 from AdHu5 is found in SEQ ID NO. 39. A corresponding nucleotide sequence is found at nucleotides 28959 to 29132 and 27969 to 28247 of SEQ ID NO. 38. In one embodiment, the vector of the present invention comprises the nucleotide sequences of AdHu5 E4Orf4, E4Orf6 and E4Orf6/7 or sequences substantially identical thereto.

In a particularly preferred embodiment of the present invention, the genome of the viral vector according to the present invention lacks the nucleotide sequences which encode the adenovirus E1 and E3 regions, and has the native E4 locus replaced with E4Orf4, E4Orf6 and E4Orf6/7 coding regions from AdHu5, and the E4Orf1, E4Orf2 and E4Orf3 coding regions from AdY25. This particularly preferred embodiment is referred to herein interchangeably as “ChAdOX1” or “AdChOX1”. As described in Example 3, and shown in FIG. 3A, the modification of the vector in this way was surprisingly found to increase the rate of hexon production and the growth and replication of the virus.

An exemplary nucleotide sequence encoding ChAdOX1 is set out in SEQ ID NO. 38.

In this embodiment, E1A, E1B 19 kDa and E1B 55 kDa are deleted and replaced with a Gateway® Destination Cassette (nucleotides 592 to 2550 of SEQ ID NO. 38). E3 CRla1, E3 gp19 kDa, E3 22.3 kDa, E3 31 kDa, E3 10.4 kDa, E3 15.2 kDa and E3 14.7 kDa are deleted and replaced with a Pac site (nucleotides 26286 to 26293 of SEQ ID NO. 38). The native E4 region is deleted and replaced with E4Orf4, E4Orf6 and E4Orf6/7 coding regions from AdHu5, and the E4Orf1, E4Orf2 and E4Orf3 coding regions from AdY25, as described above. The viral vector encoded by SEQ ID NO. 38 also comprises a number of wild-type AdY25 proteins, the nucleotide sequences of which are set out in Table 2, below:

| | Corresponding nucleotides in |
|---|---|
| Protein | SEQ ID NO. 38 |
| pIX | 2638 to 3066 |
| IVa2 | 4734 to 4749 and 3125 to 4458 |
| Polymerase | 12985 to 12993 and 4228 to 7809 |
| pTP | 12985 to 12993 and 7610 to 9539 |
| 52/55 kD | 9974 to 11164 |
| IIIa | 11188 to 12954 |
| Penton | 13038 to 14633 |
| VII | 14640 to 15221 |
| V | 15266 to 16288 |
| Mu | 16308 to 16541 |
| VI | 16617 to 17348 |
| Hexon | 17449 to 20277 |
| Endoprotease | 20293 to 20922 |
| DNA Binding Protein | 20999 to 22537 |
| 100 kDa | 22566 to 24974 |
| 22K | 24691 to 25245 |
| 33K | 24691 to 25018, 25188 . . . 25519 |
| VIII | 25602 to 26285 |
| Fiber | 26543 to 27874 |
| E4Orf3 | 29406 to 29759 |
| E4Orf2 | 29756 to 30145 |
| E4Orf1 | 30195 to 30569 |

Preferably, the genome of the viral vector according to the present invention comprises the nucleotide sequence of SEQ ID NO.38 or a sequence substantially identical thereto, into which is inserted the exogeneous nucleotide sequence encoding the protein of interest.

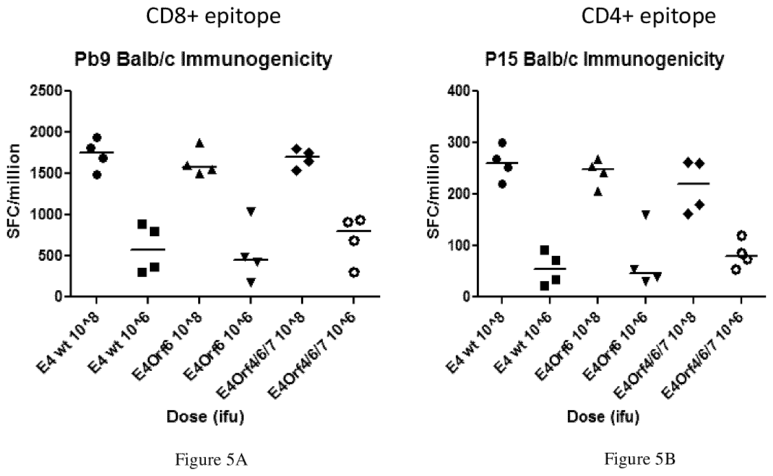

As described in Example 5 and shown in FIGS. 5A and 5B, modification of the E4 region was found to have little impact on immunogenicity of the viral vector, but did improve the rate of viral growth and replication. Therefore, such E4 modifications can be used to enhance the rate of production of the viral vectors, but will not have a negative impact on the immunogenicity of the vectors.

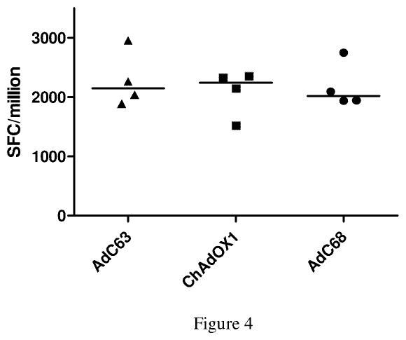

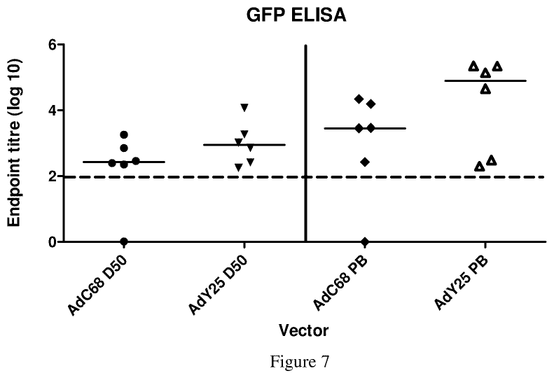

Example 4 and FIG. 4 demonstrate that the immune responses elicited by the AdY25-based vector ChAdOX1 are robust and comparable to those elicited by AdCh63 (also known as ChAd63) and AdCh68 (also known as AdC68, ChAd68, C9 or SAdV-25). However, the humoral immunogenicity of ChAdOX1 was found to be superior to that of AdCh68, as described in Example 7 and FIG. 7. One of skill in the art would expect T-cell responses and antibody responses to correlate fully with one another. The superiority of the humoral responses to ChAdOX1 is therefore surprising.

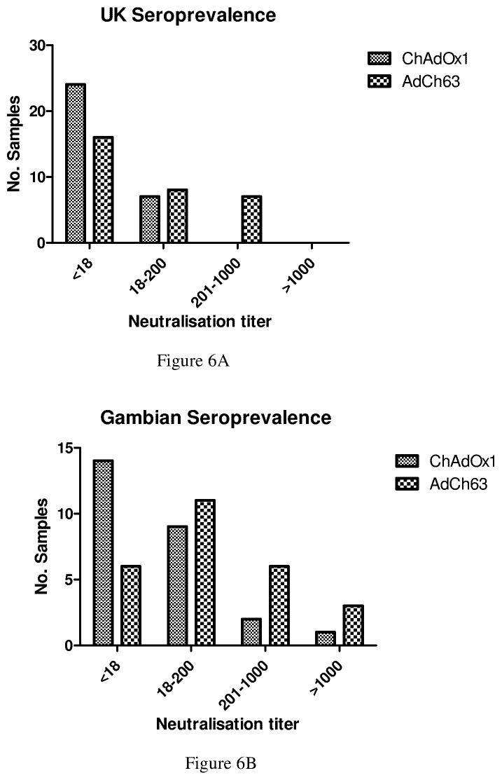

The prevalence of vector neutralising antibodies in human sera from the UK and the Gambia was also surprisingly found to be much lower for the AdY25-based vectors than for another chimpanzee adenoviral vector, AdCh63 (see Example 6 and FIGS. 6A and 6B). This data suggest that vectors based on AdY25 may encounter less pre-existing immunity within the human population, not only in comparison to vectors based on human adenoviruses, but also in comparison to other existing vectors based on chimpanzee adenoviruses.

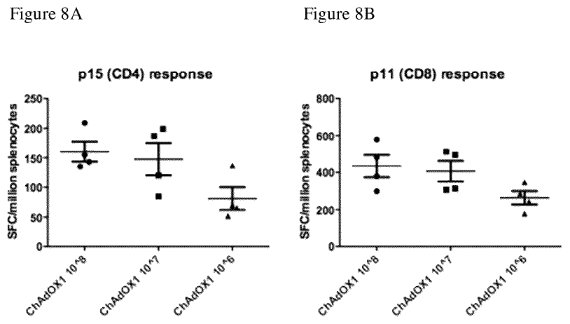

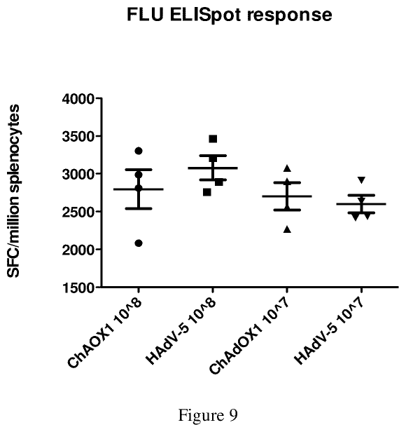

Example 8 and FIGS. 8 A and 8B demonstrate that ChAdOX1 is capable of inducing immune responses against Mycobacterium tuberculosis, whilst Example 9 and FIG. 9 demonstrate that ChAdOX1 is capable of inducing immune responses against Influenza A.

A third aspect of the present invention provides a pharmaceutical or immunogenic composition comprising the viral vector according to the second aspect of the present invention optionally in combination with one or more additional active ingredients, a pharmaceutically acceptable carrier, diluent, excipient or adjuvant.

Preferably, the composition is an immunogenic and/or antigenic composition.

The immunogenic and/or antigenic compositions according to the present invention may be prophylactic (to prevent infection), post-exposure (to treat after infection but before disease) or therapeutic (to treat disease). Preferably, the composition is prophylactic or post-exposure. Preferably, the composition is a vaccine.

Where the immunogenic composition is for prophylactic use, the subject is preferably an infant, young child, older child or teenager. Where the immunogenic composition is for therapeutic use, the subject is preferably an adult.

The composition may comprise one or more additional active agents, such as an anti-inflammatory agent (for example a p38 inhibitor, glutamate receptor antagonist, or a calcium channel antagonist), AMPA receptor antagonist, a chemotherapeutic agent and/or an antiproliferative agent. The composition may also comprise one or more antimicrobial compounds. Examples of suitable antimicrobial compounds include antituberculous chemotherapeutics such as rifampicin, isoniazid, ethambutol and pyrizinamide.

Suitable carriers and/or diluents are well known in the art and include pharmaceutical grade starch, mannitol, lactose, magnesium stearate, sodium saccharin, talcum, cellulose, glucose, sucrose, (or other sugar), magnesium carbonate, gelatin, oil, alcohol, detergents, emulsifiers or water (preferably sterile). The composition may be a mixed preparation of a composition or may be a combined preparation for simultaneous, separate or sequential use (including administration).

Suitable adjuvants are well known in the art and include incomplete Freund's adjuvant, complete Freund's adjuvant, Freund's adjuvant with MDP (muramyldipeptide), alum (aluminium hydroxide), alum plus Bordatella pertussis and immune stimulatory complexes (ISCOMs, typically a matrix of Quil A containing viral proteins).

The composition according to the invention for use in the aforementioned indications may be administered by any convenient method, for example by oral (including by inhalation), parenteral, mucosal (e.g. buccal, sublingual, nasal), rectal or transdermal administration and the compositions adapted accordingly.

For oral administration, the composition can be formulated as liquids or solids, for example solutions, syrups, suspensions or emulsions, tablets, capsules and lozenges.

A liquid formulation will generally consist of a suspension or solution of the compound or physiologically acceptable salt in a suitable aqueous or non-aqueous liquid carrier(s) for example water, ethanol, glycerine, polyethylene glycol or oil. The formulation may also contain a suspending agent, preservative, flavouring or colouring agent.

A composition in the form of a tablet can be prepared using any suitable pharmaceutical carrier(s) routinely used for preparing solid formulations. Examples of such carriers include magnesium stearate, starch, lactose, sucrose and microcrystalline cellulose.

A composition in the form of a capsule can be prepared using routine encapsulation procedures. For example, powders, granules or pellets containing the active ingredient can be prepared using standard carriers and then filled into a hard gelatine capsule; alternatively, a dispersion or suspension can be prepared using any suitable pharmaceutical carrier(s), for example aqueous gums, celluloses, silicates or oils and the dispersion or suspension then filled into a soft gelatine capsule.

Compositions for oral administration may be designed to protect the active ingredient against degradation as it passes through the alimentary tract, for example by an outer coating of the formulation on a tablet or capsule.

Typical parenteral compositions consist of a solution or suspension of the compound or physiologically acceptable salt in a sterile aqueous or non-aqueous carrier or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration.

Compositions for nasal or oral administration may conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomising device. Alternatively the sealed container may be a unitary dispensing device such as a single dose nasal inhaler or an aerosol dispenser fitted with a metering valve, which is intended for disposal once the contents of the container have been exhausted. Where the dosage form comprises an aerosol dispenser, it will contain a pharmaceutically acceptable propellant. The aerosol dosage forms can also take the form of a pump-atomiser.

Compositions suitable for buccal or sublingual administration include tablets, lozenges and pastilles, wherein the active ingredient is formulated with a carrier such as sugar and acacia, tragacanth, or gelatin and glycerin.

Compositions for rectal or vaginal administration are conveniently in the form of suppositories (containing a conventional suppository base such as cocoa butter), pessaries, vaginal tabs, foams or enemas.

Compositions suitable for transdermal administration include ointments, gels, patches and injections including powder injections.

Conveniently the composition is in unit dose form such as a tablet, capsule or ampoule.

The pharmaceutical composition is preferably sterile. It is preferably pyrogen-free. It is preferably buffered e.g. at between pH 6 and pH 8, generally around pH 7.

Preferably, the composition is substantially isotonic with humans.

Preferably, the pharmaceutical compositions of the present invention deliver an immunogenically or pharmaceutically effective amount of the viral vector to a patient.

As used herein ‘immunogenically or pharmaceutically effective amount’ means that the administration of that amount to an individual, either as a single dose or as a series of doses, is effective for prevention or treatment of a disease or condition. In particular, this phrase means that a sufficient amount of the viral vector is delivered to the patient over a suitable timeframe such that a sufficient amount of the antigen is produced by the patient's cells to stimulate an immune response which is effective for prevention or treatment of a disease or condition. This amount varies depending on the health and physical condition of the individual to be treated, age, the capacity of the individual's immune system, the degree of protection desired, the formulation of the vaccine, the doctor's assessment of the medical situation and other relevant factors.

In general, a pharmaceutically effective dose comprises 1×10⁷ to 1×10¹² viral particles, preferably 1×10¹⁰ to 1×10¹¹ particles.

The immunogenic composition of the present invention may also comprise one or more other viral vectors, preferably other adenoviral vectors.

A fourth aspect of the present invention provides the use of the viral vector according to the second aspect of the present invention or the immunogenic composition according to the third aspect of the present invention. In particular, the fourth aspect provides the use of the viral vector or the immunogenic composition of the present invention in medicine.

This aspect also provides: i) the viral vector or the immunogenic composition according to the present invention for use in medicine and ii) the use of the viral vector or the immunogenic composition according to the present invention in the manufacture of a medicament for use in medicine. Some exemplary medical uses are described in further detail below.

In one embodiment, the viral vector according to the second aspect of the present invention or the immunogenic composition according to the third aspect of the present invention may be used to deliver a transgene into a host cell.

This method preferably comprises the step of administering to said host cell a viral vector according to the second aspect of the present invention or the immunogenic composition according to the third aspect of the present invention.

Preferably, the host cell is an animal cell, more preferably a mammalian cell.

Preferred mammals include chickens, other poultry, cows, sheep, goats, pigs, wild boar, buffalo, bison, horses, camelids, deer, elephants, badgers, possums, cats, lions, monkeys and humans. Preferably, the host cell is a somatic cell. The host cell may be selected from the group consisting of an antigen-presenting dendritic cell, langerhans cell, macrophage, B cell, lymphocyte, leukocyte, myocyte and fibroblast.

This method may be carried out in vitro or in vivo. Where the method is carried out in vitro, the viral vector or immunogenic composition is brought into contact with the host cell under suitable conditions such that transduction or non-productive infection of the host cell with the viral vector is facilitated. In this embodiment, the host cell may comprise an isolated host cell or a sample from an animal subject. Where the method is carried out in vivo, the viral vector or immunogenic composition is preferably administered to the animal subject such that transduction of one or more cells of the subject with the viral vector is facilitated. Preferably, the viral vector or immunogenic composition is administered to the subject by oral (including by inhalation), parenteral, mucosal (e.g. buccal, sublingual, nasal), rectal or transdermal administration.

Preferably, the transduction of the host cell with the viral vector of the present invention results in the stable delivery of the exogeneous nucleotide sequence of interest into the host cell.

Therefore, in another embodiment, the viral vector according to the second aspect of the present invention or the immunogenic composition according to the third aspect of the present invention may be used to elicit an immune response in an animal. This method preferably comprises the step of administering to said animal a viral vector according to the second aspect of the present invention or the immunogenic composition according to the third aspect of the present invention.

Where the protein or polypeptide of interest is an antigen, expression of the protein or polypeptide in an animal will result in the elicitation of a primary immune response to that antigen, leading to the development of an immunological memory which will provide an enhanced response in the event of a secondary encounter, for example upon infection by the pathogen from which the antigen was derived.

Preferably, the animal is a naive animal, i.e. an animal that has not previously been exposed to the pathogen or antigens in question.

As well as eliciting an immune response in an animal, the viral vector of the present invention or the immunogenic composition thereof can be used to boost the immune response of an animal previously exposed to the antigen.

Therefore, in a further embodiment, the viral vector according to the second aspect of the present invention or the immunogenic composition according to the third aspect of the present invention may be used to boost an immune response in an animal. This method preferably comprises the step of administering to said animal a viral vector according to the second aspect of the present invention or the immunogenic composition according to the third aspect of the present invention.

Preferably, the animal subject has been previously exposed to the antigen in question, or “primed”. For example, the subject may have previously been inoculated or vaccinated with a composition comprising the antigen, or may have previously been infected with the pathogen from which the antigen was derived. The subject may be latently infected with the pathogen from which the antigen was derived.

In another embodiment, the vector according to the second aspect of the present invention or the immunogenic composition according to the third aspect of the present invention may be used to treat or prevent at least one disease in a patient. This method preferably comprising the step of administering to said patient a viral vector according to the second aspect of the present invention or the immunogenic composition according to the third aspect of the present invention.

Preferably, the disease is selected from the group consisting of Tuberculosis and other mycobacterial infections, malaria, influenza, HIV/AIDS, Hepatitis C, Cytomegalovirus infection, Human papilloma virus infection, adenoviral infection, leishmaniasis, streptococcus spp., staphylococcus spp., meningococcus spp., infection, rift valley fever, foot and mouth disease and chikungunya virus infection.

As well as inducing an immune response against the pathogenic organism from which the heterologous antigen is derived, the adenoviral vector of the present invention may also induce an immune response against the adenovirus from which the viral vector is derived. As such, an immune response against AdY25 may be elicited. The immune response induced against AdY25 may also be cross-reactive with other adenoviral serotypes, and as such an immune response against more than one adenovirus may be elicited. The viral vector according to the second aspect of the present invention or the immunogenic composition according to the third aspect of the present invention can therefore also be used for treating or preventing an adenoviral disease.

This embodiment of the present invention therefore also provides the treatment or prevention of at least one adenoviral disease and at least one non-adenoviral disease in a patient.

In a further embodiment, the viral vector according to the second aspect of the present invention or the immunogenic composition according to the third aspect of the present invention may be used to induce an immune response in an animal that will break tolerance to a self antigen. This method preferably comprises the step of administering to said animal a viral vector according to the second aspect of the present invention or the immunogenic composition according to the third aspect of the present invention.

Many tumour cells are tolerated by the patient's immune system, on the grounds that tumour cells are essentially the patient's own cells that are growing, dividing and spreading without proper regulatory control. Thus, cancerous tumours are able to grow unchecked within the patient's body. However, the viral vector of the present invention can be used to stimulate a patient's immune system to attack the tumour cells in a process known as “cancer immunotherapy”. Specifically, the vector of the present invention can be used to ‘train’ the patient's immune system to recognise tumour cells as targets to be destroyed. This can be achieved by including within the viral vector an exogeneous nucleotide sequence encoding a suitable self-antigen. As described previously, suitable self-antigens include antigens expressed by tumour cells which allow the immune system to differentiate between tumour cells and other cell types. Suitable self-antigens include antigens that are either inappropriate for the cell type and/or its environment, or are only normally present during the organisms' development (e.g. foetal antigens). For example, GD2 is normally only expressed at a significant level on the outer surface membranes of neuronal cells, where its exposure to the immune system is limited by the blood-brain barrier. However, GD2 is expressed on the surfaces of a wide range of tumour cells including small-cell lung cancer, neuroblastoma, melanomas and osteosarcomas. Other suitable self-antigens include cell-surface receptors that are found on tumour cells but are rare or absent on the surface of healthy cells. Such receptors may be responsible for activating cellular signalling pathways that result in the unregulated growth and division of the tumour cell. For example, ErbB2 is produced at abnormally high levels on the surface of breast cancer tumour cells. Thus, the adenoviral vector of the present invention may be used to induce an immune response against a tumour cell, and can therefore be used in the treatment of cancer.

The following details apply mutatis mutandis to all of the above uses of the vector and immunogenic composition of the present invention.

The treatment and prevention of many diseases, including liver stage malaria, tuberculosis and influenza, are associated with the maintenance of a strong cell-mediated response to infection involving both CD4+ and CD8+ T cells and the ability to respond with Th1-type cytokines, particularly IFN-γ, TNF-α, IL-2 and IL-17. Although many subunit vaccine platforms effectively generate human immunity, the generation of robust cell-mediated immune responses, particularly CD4+ and CD8+ T cell immune responses, has been much more challenging. The viral vector of the present invention preferably stimulates both cellular and humoral immune responses against the encoded antigen.

It is also desirable to induce a memory immune response. Memory immune responses are classically attributed to the reactivation of long-lived, antigen-specific T lymphocytes that arise directly from differentiated effector T cells and persist in a uniformly quiescent state. Memory T cells have been shown to be heterogeneous and to comprise at least two subsets, endowed with different migratory capacity and effector function; effector memory T cells (TEM) and central memory T cells (CTM). TEM resemble the effector cells generated in the primary response in that they lack the lymph node-homing receptors L-selectin and CCR7 and express receptors for migration into inflamed tissues. Upon re-encounter with antigen, these TEM can rapidly produce IFN-7 or IL-4 or release pre-stored perform. TCM express L-selectin and CCR7 and lack immediate effector function. These cells have a low activation threshold and, upon restimulation in secondary lymphoid organs, proliferate and differentiate to effectors.

Preferably, the viral vector according to the second aspect of the present invention or the immunogenic composition according to the third aspect of the present invention is capable of eliciting, inducing or boosting an antigen-specific immune response.

Preferably, the immune response is a strong T cell immune response, for example a strong CD8+ and CD4+ T cell response. Preferably, the T cell immune response is a

protective T cell immune response. Preferably, the T cell immune response is long lasting and persists for at least 1, 2, 5, 10, 15, 20, 25 or more years. Preferably, the immune response induced is a memory T cell immune response.

The viral vector of the second aspect of the present invention or immunogenic composition according to the third aspect of the present invention may be administered to the host cell or subject either as a single immunisation or multiple immunisations. Preferably, the viral vector or immunogenic composition thereof are administered as part of a single, double or triple vaccination strategy. They may also be administered as part of a homologous or heterologous prime-boost immunisation regime.

The vaccination strategy or immunisation regime may include second or subsequent administrations of the viral vector or immunogenic composition of the present invention. The second administration can be administered over a short time period or over a long time period. The doses may be administered over a period of hours, days, weeks, months or years, for example up to or at least 1, 2, 3, 4, 5, 6, 7, 8, 9, or 10 or more weeks or 0.25, 0.5, 0.75, 1, 5, 10, 15, 20, 25, 30, 35 or 40 or more years after the first administration. Preferably, the second administration occurs at least 2 months after the first administration. Preferably, the second administration occurs up to 10 years after the first administration. These time intervals preferably apply mutatis mutandis to the period between any subsequent doses.

The viral vector and/or immunogenic composition may be administered alone or in combination with other viral or non-viral DNA/protein vaccines. Preferred examples include MVA, FP9 and other adenoviral vector vaccines.

The viral vector and/or immunogenic composition may be administered to the subject by oral (including by inhalation), parenteral, mucosal (e.g. buccal, sublingual, nasal), rectal or transdermal administration. Alternatively, the viral vector and/or immunogenic composition may be administered to an isolated host cell or sample from a subject by contacting the cell(s) with the viral vector or immunogenic composition in vitro under conditions that facilitate the transduction of the host cell with the viral vector.

The viral vector and immunogenic composition of the present invention are not limited to the delivery of nucleic acid sequences encoding antigens. Many diseases, including cancer, are associated with one or more deleterious mutant alleles in a patient's genome. Gene therapy is a process involving the insertion of genes into the patient's cells or tissues to replace the deleterious mutant or non-functional allele(s) with ‘normal’ or functional allele(s). Commonly, a functional allele is inserted into a non-specific location within the genome to replace the non-functional allele.

Alternatively, the non-functional allele may be swapped for the functional allele through homologous recombination. Subsequent expression of the functional allele within the target cell restores the target cell to a normal state and thus provides a treatment for the disease. The ‘normal’ or functional allele(s) may be inserted into a patient's genome using a viral vector. The present invention therefore also provides the use of the viral vector according to the second aspect of the present invention or the immunogenic composition according to the third aspect of the present invention in gene therapy.

This method preferably comprises the step of administering to said animal a viral vector according to the second aspect of the present invention or the immunogenic composition according to the third aspect of the present invention.

The vector of the present invention may comprise an exogeneous nucleotide sequence encoding the functional or ‘normal’ protein, the non-functional or ‘mutant’ version of which is associated with a disease or condition.

Preferably, the target cell is a somatic cell. The subject to be treated is preferably mammalian. Preferred mammals include chickens, other poultry, cows, sheep, goats, pigs, wild boar, buffalo, bison, horses, camelids, deer, elephants, badgers, possums, cats, lions, monkeys and humans.

A fifth aspect of the present invention provides a polynucleotide sequence encoding the viral vector according to the second aspect of the present invention. Preferably, the polynucleotide sequence comprises the sequence of SEQ ID NO. 38 or a sequence substantially identical thereto. The polynucleotide may additionally comprise the exogeneous nucleotide sequence of interest.

A sixth aspect of the present invention provides a host cell transduced or infected with the viral vector according to the second aspect of the present invention. Following transduction or infection, the host cell will express the exogeneous nucleotide sequence in the nucleic acid molecule to produce the molecule of interest, in addition to any other adenoviral proteins encoded by the nucleic acid molecule. Preferably, the host cell is stably transduced and suitable for viral propagation.

The host cell may be an isolated host cell, part of a tissue sample from an organism, or part of a multicellular organism or organ or tissue thereof.

Preferably, the host cell is a somatic cell. Preferably, the host cell is not a stem cell, more particularly an embryonic stem cell, more particularly a human embryonic stem cell.

The host cell may be selected from the group consisting of an antigen-presenting dendritic cell, langerhans cell, macrophage, B cell, lymphocyte, leukocyte, myocyte and fibroblast.

Preferably, the host cell is an animal cell, more preferably a mammalian cell.

Preferred mammals include chickens, other poultry, cows, sheep, goats, pigs, wild boar, buffalo, bison, horses, camelids, deer, elephants, badgers, possums, cats, lions, monkeys and humans.

The fifth aspect of the present invention also encompasses an animal transduced or infected with the viral vector according to the second aspect of the present invention. Preferably, the animal comprises one or more cells transformed or transfected with the viral vector according to the second aspect of the present invention. Preferably, the animal is a mammal. Preferred mammals include chickens, other poultry, cows, sheep, goats, pigs, wild boar, buffalo, bison, horses, camelids, deer, elephants, badgers, possums, cats, lions, monkeys and humans.

In a seventh aspect, the present invention provides a method of producing the viral vector according to the second aspect of the present invention. Preferably, the method comprises the step of incorporating a nucleotide sequence derived from AdY25 into a Bacterial Artificial Chromosome (BAC) to produce an Ad-BAC vector.

Unlike plasmid vectors, BACs are present within E. Coli in single copy conferring increased genetic stability. In addition, the single copy BAC vectors permit very precise modifications to be made to the viral genome by recombineering (recombination mediated genetic engineering).

Preferably, incorporation of the nucleotide sequence derived from AdY25 into a Bacterial Artificial Chromosome (BAC) comprises the steps of:
constructing a BAC rescue vector comprising regions of homology to the left and right flanks of the viral nucleotide sequence;
linearising the BAC rescue vector; and
performing homologous recombination in a host cell between the viral nucleotide sequence and the linearised BAC rescue vector to incorporate the viral nucleotide sequence into the BAC rescue vector.

Preferably, the nucleotide sequence incorporated into the BAC rescue vector comprises the sequence of SEQ ID NO. 1 or SEQ ID NO. 38 or a sequence substantially identical thereto.

Preferably, the method additionally comprises the step of further modifying the Ad-BAC vector genome. These further modifications may be carried out by GalK recombineering. This technique, pioneered by Saren Warming and colleagues, utilises the GalK gene for both positive and negative selection of recombinant clones6. SW102 E. Coli cells, in which recombination may be performed, have been specifically engineered to lack the GalK gene which is required for the utilisation of galactose as the sole carbon source. Gene deletion is performed by recombination between the vector genome and a PCR amplified GalK cassette, flanked by 50 bp regions of homology either side of the gene targeted for deletion. Selection on minimal media containing only galactose should ensure that only recombinants containing the GalK gene (in place of the target gene) should grow. Replacement of GalK with a different gene sequence can be performed in a similar fashion, this time using GalK for negative selection. The addition of 2-deoxygalactose (DOG) to selection media will select clones in which GalK has been replaced since the product of GalK, galactokinase, metabolises DOG into a product that is highly toxic to E. Coli. Preferably, the host cell is BJ5183 E. Coli for steps i) to iii) above and SW102 for further modifications.

Preferably, an extra homology flank is included downstream of the adenovirus E1 region to enable simultaneous deletion of E1, as described in Example 1.

Preferably, the method further includes deletion of the E3 region of the Ad-BAC vector genome. Deletion of the E3 region may be carried out by GalK recombineering, as described in Example 2.

Preferably, the method further includes modifying the E4 region to optimise vector growth and yield. In one embodiment, the entire native E4 locus is replaced with the E4Orf6 gene from AdHu5. In a second embodiment, the native E4 locus is replaced with E4Orf4, E4Orf6 and E4Orf6/7 coding regions from AdHu5, and the E4Orf1, E4Orf 2 and E4Orf 3 coding regions from AdY25, as described in Example 3.

Preferably, the method further includes introducing phage lambda site specific recombination sites attR1 and attR2 at the Ad E1 locus as part of an Invitrogen Gateway® destination cassette. Such a modification enables the efficient directional insertion of vaccine transgenes. Transgenes could also be inserted by recombineering, In-Fusion®, conventional ligation or gap repair.

An eighth aspect of the present invention provides a Bacterial Artificial Chromosome (BAC) clone comprising a polynucleotide sequence encoding the viral vector according to the second aspect of the present invention.

Preferably, the BAC clone comprises:
a. a BAC backbone;
b. the polynucleotide sequence according to the fifth aspect of the present invention.

As described above, the viral vector according to the second aspect of the present invention may be replicated in a transformed cell line or helper virus (gutless vector system) which, if necessary, comprises the complement of any genes deleted from the virus. Such genes may be deleted from the virus in order to hinder replication in host cells, but are of course required in order to replicate the viral vector to produce immunogenic compositions according to the second aspect of the present invention. One can make use of any cell line permissive of wild type adenovirus replication that has been modified to express the functionally deleted genes, or a cell line which is not permissive of wild-type virus replication which has additionally or alternatively been modified to express CAR or integrins in addition to the functionally deleted genes.

The present invention provides host cells comprising a Bacterial Artificial Chromosome (BAC) in accordance with the eighth aspect of the present invention, and suitable for propagation thereof. Preferably such host cells are bacteria, most preferably E. coli. Suitable examples include E. coli strains DH10B and SW1029.

A ninth aspect of the present invention therefore provides a packaging cell or cell line producing or capable of producing the viral vector according to the second aspect of the present invention. The packaging cell or cell line comprises one or more nucleotide sequences which encode the viral vector of the second aspect of the present invention. Expression of these sequences results in the production of the viral vector. Some of the required genes may be provided by infection of the cell or cell line with a viral vector according to the second aspect. Preferably, the cell comprises the complement of any genes deleted or functionally deleted from the viral vector. Preferably, the cell comprises the complement of the AdY25 E1 gene. Preferably, the cell is an HEK293 cell or a PER.C6® cell.

As described above, modification of the E4 locus of the adenoviral vector to include the E4Orf4, E4Orf6 and E4Orf6/7 coding regions from AdHu5 increased the rate of hexon production, increasing the sensitivity of anti-hexon titre to allow quantification of the infectious titre of the viral vector, in particular those viral vectors developed for clinical use which do not contain a fluorescent marker gene. In addition, this modification was surprisingly found to increase the yield and rate of growth of the vector. One of skill in the art would appreciate that such a modification is expected to have a beneficial effect on a wide variety of adenoviruses, and not simply those derived from AdY25.

A tenth aspect of the present invention therefore provides an adenoviral vector other than AdHu5 comprising a nucleic acid molecule, wherein said nucleic acid molecule comprises heterologous E4Orf4, E4Orf6 and E4Orf6/7 coding regions from AdHu5.

In one embodiment, the native E4 locus is deleted and replaced with heterologous E4Orf4, E4Orf6 and E4Orf6/7 coding regions from AdHu5. Alternatively, nucleic acid molecule may comprise the native coding regions in addition to heterologous E4Orf4, E4Orf6 and E4Orf6/7 coding regions from AdHu5. Preferably, the native coding regions are E4Orf1, E4Orf2 and E4Orf3.

Preferred adenoviral vectors are selected from the group consisting of AdY25 and AdY68.

Preferably, the adenoviral vector according to the tenth aspect lacks and E1 and an E3 locus.

Merely for the convenience of those of skill in the art, a sample of E. coli strain SW1029 (a derivative of DH10B) containing bacterial artificial chromosomes (BACs) containing the cloned genome of AdChOX1 (pBACe3.6 AdChOx1 (E4 modified) TIPeGFP, cell line name “AdChOx1 (E4 modified) TIPeGFP”) was deposited by Isis Innovation Limited on 24 May 2012 with the European Collection of Cell Cultures (ECACC) at the Health Protection Agency Culture Collections, Health Protection Agency, Porton Down, Salisbury SP4 0JG, United Kingdom under the Budapest Treaty and designated by provisional accession no. 12052403.

As described herein, the vector AdChOx1 is derived from chimpanzee adenovirus Y25, with deletion of E1 region, E3 region, modification of E4 region and insertion of TIPeGFP model antigen into E1 locus. The E. coli containing the BAC is a class I genetically modified organism.

The BAC propagates within the bacteria during replication and can be maintained by selection with chloramphenicol. The E. coli strain SW102 containing the bacterial artificial chromosomes into which the genomes are cloned can be propagated in Luria-Bertani broth or agar containing 12.5 g/mL chloramphenicol at 32° C. The genome may be modified by genetic engineering in E. coli according to standard methods, as described in the specification, e.g. to insert an alternative recombinant antigen in place of TIPeGFP.

Converting the BAC clones of the viral genomes into viruses (“rescue”) can be carried out by the following steps. The E. coli host is propagated and the BAC DNA is purified from the bacteria according to standard methods. The DNA is linearised with the restriction endonuclease PmeI and transfected into HEK293 cells (or a similar E1 complementing cell line). The resulting adenovirus can then be propagated and purified for use as a vaccine, for example. All of these reagents and cells are publicly available. If the deposition were rescued, the resulting virus would be a class I genetically modified organism.

In respect of all designated states to which such action is possible and to the extent that it is legally permissible under the law of the designated state, it is requested that a sample of the deposited material be made available only by the issue thereof to an independent expert, in accordance with the relevant patent legislation, e.g. Rule 32(1) EPC, Rule 13(1) and Schedule 1 of the UK Patent Rules 2007, Regulation 3.25(3) of the Australian Patent Regulations and generally similar provisions mutatis mutandis for any other designated state.

A specific embodiment of the fifth aspect of the present invention provides a polynucleotide sequence encoding an adenoviral vector according to the second aspect of the present invention, wherein said polynucleotide sequence comprises or consists of the polynucleotide sequence of the viral vector AdChOX1, deposited in a BAC contained in E. coli strain SW1029 by Isis Innovation Limited on 24 May 2012 with the European Collection of Cell Cultures (ECACC) at the Health Protection Agency Culture Collections, Health Protection Agency, Porton Down, Salisbury SP4 0JG, United Kingdom under the Budapest Treaty and designated by provisional accession no. 12052403. The deposited BAC additionally comprises a transgene encoding the antigen TIPeGFP. In this aspect of the present invention, the polynucleotide sequence for AdChOX1 preferably does not include the sequence encoding the TIPeGFP antigen.

A further embodiment of the present invention provides a host cell transduced with the viral vector according to the second aspect of the present invention, wherein said host cell is preferably a bacterium, more preferably E. coli strain SW1029 containing a bacterial artificial chromosome (BAC) containing the cloned genome of AdChOX1 deposited by Isis Innovation Limited on 24 May 2012 with the European Collection of Cell Cultures (ECACC) at the Health Protection Agency Culture Collections, Health Protection Agency, Porton Down, Salisbury SP4 0JG, United Kingdom under the Budapest Treaty and designated by provisional accession no. 12052403. The deposited BAC additionally comprises a transgene encoding the antigen TIPeGFP. In this aspect of the present invention, the polynucleotide sequence for AdChOX1 preferably does not include the sequence encoding the TIPeGFP antigen. Such a host cell may be used for BAC propagation.

A specific embodiment of the seventh aspect of the present invention provides a method of producing the viral vector according to the second aspect of the present invention by generating a molecular clone of AdY25 in a Bacterial Artificial Chromosome (BAC), wherein said BAC is the BAC containing the cloned genome of AdChOX1, deposited in E. coli strain SW1029 by Isis Innovation Limited on 24 May 2012 with the European Collection of Cell Cultures (ECACC) at the Health Protection Agency Culture Collections, Health Protection Agency, Porton Down, Salisbury SP4 0JG, United Kingdom under the Budapest Treaty and designated by provisional accession no. 12052403. The deposited BAC additionally comprises a transgene encoding the antigen TIPeGFP. In this aspect of the present invention, the polynucleotide sequence for AdChOX1 preferably does not include the sequence encoding the TIPeGFP antigen.

A specific embodiment of the eighth aspect of the present invention provides a Bacterial Artificial Chromosome (BAC) clone comprising the polynucleotide sequence according to the fifth aspect of the present invention, wherein said BAC is the BAC containing the cloned genome of AdChOX1, deposited in E. coli strain SW1029 by Isis Innovation Limited on 24 May 2012 with the European Collection of Cell Cultures (ECACC) at the Health Protection Agency Culture Collections, Health Protection Agency, Porton Down, Salisbury SP4 0JG, United Kingdom under the Budapest Treaty and designated by provisional accession no. 12052403. The deposited BAC additionally comprises a transgene encoding the antigen TIPeGFP. In this aspect of the present invention, the polynucleotide sequence for AdChOX1 preferably does not include the sequence encoding the TIPeGFP antigen.

For the avoidance of doubt, it is hereby expressly stated that features described herein as ‘preferred’, ‘preferable’, “alternative” or the like may be present in the invention in isolation or in any combination with any one or more other features so described (unless the context dictates otherwise) and this constitutes and explicit disclosure of such combinations of features.

All the features of each embodiment described above apply mutatis mutandis to all other embodiments of the present invention.

### EXAMPLES

### Example 1. Generation of a Molecular Clone of AdY25 in a Bacterial Artificial

Chromosome Wild type chimpanzee adenovirus AdY25 was obtained from Goran Wadell of Umea University, Sweden. The virus was propagated to high titer in HEK293 cells and the viral DNA phenol extracted and sequenced. The nucleotide sequence of the wild type AdY25 virus is found in SEQ ID NO. 1. Based on the sequencing data, a BAC ‘rescue vector’ was constructed containing regions of homology to the left and right flanks of the viral genome (homology flanks were PCR amplified from viral DNA). Homologous recombination was then performed in BJ5183 E. Coli cells between viral DNA and the linearised rescue vector to incorporate the viral genome into the BAC vector.

An extra homology flank downstream of the adenovirus E1 region was included to enable simultaneous deletion of E1 in order to render the new vector immediately replication incompetent.

Phage lambda site specific recombination sites attR1 and attR2 were introduced at the Ad E1 locus as part of an Invitrogen Gateway® destination cassette to enable the efficient directional insertion of vaccine transgenes. A modified destination cassette was ligated into the AsiSI restriction site introduced at the E1 locus during isolation of the genomic clone.

The resulting ΔE1 Ad-BAC vector was screened by both PCR and restriction digest before replication incompetent clones were transfected into E1 complementing HEK293 cells, where the new vector demonstrated the ability to produce infectious virions capable of replication and cytopathic effect in HEK293 cells.

### Example 2: Deletion of the adenoviral E3 region

The ΔE1 Ad-BAC vector genome produced in accordance with Example 1 was further modified using GalK recombineering to delete the adenoviral E3 region and thus increase the insert capacity of the new vector by approximately 5 kb.

The E3 region was deleted by recombination between the vector genome and a PCR amplified GalK cassette, flanked by 50 bp regions of homology either side of the E3 gene. Recombination was performed in SW102 E. coli cells, which have been specifically engineered to lack the GalK gene which is required for the utilisation of galactose as the sole carbon source. Recombinant cells were selected using minimal media containing only galactose, in which only recombinants containing the GalK gene in place of the E3 locus were able to grow6.

### Example 3: Modification of the E4 Region and Effects Thereof

### i). Modification of E4 Region

The E4 locus of the ΔE1 ΔE3 Ad-BAC vector genome produced in accordance with Example 2 was then modified. The E4 region was deleted by recombination in SW102 E. Coli cells between the vector genome and a PCR-amplified GalK cassette, flanked by 50 bp regions of homology either side of the E4 gene. Recombinant cells were selected using minimal media containing only galactose. The GalK gene was then replaced with the required E4 open reading frames from AdHu5 and AdY25 in a similar manner to provide the 5 constructs listed in FIG. 3C. Recombinant cells comprising the gene in place of the GalK gene were then selected using media comprising 2-deoxygalactose (DOG)6.

### ii). Effect of E4 Modification on Viral Yield

HEK293 cells were infected with the following viral vectors at a multiplicity of intention of 9 and incubated at 37° C. for 48 hours before harvesting:
i. AdHu5 (“Ad5”)
ii. AdY25 E4 wildtype (“Y25E4 wt”)
iii. AdY25 E4 AdHu5 E4Orf 6 (“Y25Ad5E4Orf”)
iv. AdY25 E4 AdHu5 E4Orf 4, 6, 6/7 (“AdChOX1”)

Infectious titre of the harvested material was measured by quantifying GFP positive foci 48 hours post infection.

As can be seen in FIG. 3A, the infectious titre of the AdY25-based viral vector comprising the wildtype E4 locus was significantly lower than that of AdHu5. Modification of the viral vector to replace the wildtype E4 locus with the E4Orf6 gene from AdHu5 significantly increased the infectious titre. Replacement of the wildtype E4 locus with the E4Orf4, E4Orf6 and E4Orf6/7 coding regions from AdHu5, and the E4Orf1, E4Orf2 and E4Orf3 coding regions from AdY25 (to create ChAdOX1) surprisingly further increased the infectious titre.

iii). GFP Vs. Anti-Hexon Titre

In order to assess vaccine vector immunogenicity and efficacy it is essential to develop a reliable method of quantifying the infectious titer of the virus. Traditionally, plaque assays in HEK293 cells have been the method of choice, but these require a long incubation period and titers are often inconsistent. Furthermore the plaque assay is inherently insensitive, not all infectious virions will induce plaque formation. One method is the single cell infectivity assay which simply involves quantifying the number of virally infected cells. The first recombinant AdY25-derived viral vectors expressed green fluorescent protein (GFP), enabling viruses that had initiated recombinant transgene expression within a cell to be visualised directly by fluorescence microscopy. However, an alternative method of assessing cell infectivity must be used where the vaccine antigen constructs do not contain a fluorescent marker gene, for example where the vaccine antigen constructs are for clinical use.

An anti-hexon immunostaining assay has now been developed that enables visualisation of infected cells in which the viral hexon protein is being expressed. This assay uses a polyclonal anti-hexon antibody so can be used to titer virtually any adenovirus vaccine vector and we have found the assay to be reliable and consistent for both AdHu5 and AdCh63 based vectors. It does of course rely on the assumption that the rate of hexon production relative to transgene expression is consistent between vectors. The titers of GFP-expressing AdY25-derived viral vectors were compared by GFP and anti-hexon based assays. Titers were assessed at 48 hours post infection for AdHu5, AdC63, AdY25 E4 wildtype, and constructs A-E as described in FIG. 3C, all expressing the TIPeGFP antigen.

TIP is essentially an epitope string consisting of a number of strong murine T cell epitopes including Pb9 (a dominant CD8+ epitope from malarial antigen PbCSP) and P15 (a strong CD4+ epitope from M. tuberculosis antigen 85A). The TIP epitope string is fused to the 5′ end of eGFP which enables transgene expression to be visualised directly and simplifies vaccine titration.

FIG. 3B illustrates the ratio of GFP foci to anti-hexon titer. For Ad5- and AdC63-based vectors, GFP titers were approximately twice as sensitive as anti-hexon titers. However, for AdY25-based vectors, the sensitivity of the anti-hexon assay varied considerably with E4 modification. For the AdY25 E4 wildtype vector, anti-hexon titers were over 40 fold less sensitive than GFP titers after 48 hrs, suggesting that the rate of hexon production is considerably slower than for AdHu5 and AdCh63 vectors. This was to be expected, given the poor yield of AdY25 E4 wildtype vector. Surprisingly, however, the construct A (“Y25Ad5E4Orf6”) was still 30 fold less sensitive by anti-hexon than by GFP. The best results were obtained with construct E (“ChAdOX1”), in which the wildtype E4 locus was replaced with the E4Orf4, E4Orf6 and E4Orf6/7 coding regions from AdHu5, and the E4Orf1, E4Orf2 and E4Orf3 coding regions from AdY25.

### iv). Hexon Expression

The ratio of marker gene to hexon titre for ChAdOX1 viral vectors expressing TIPeGFP was measured using GFP and mCherry fluorescent transgenes in order to control for the sensitivity of the fluorescent detection.

The results are provided in FIG. 3D. In both cases, the marker generhexon titre ratio was approximately twofold, and thus the particular marker gene used did not affect the resulting marker gene:hexon titre ratio. The marker gene:hexon titre ratio for the ChAdOX1 vector is the same as that for HAdV-5, indicating that the E4 modification to the ChAdOX1 vector has been optimised.

### Example 4: Immunogenicity of AdY25-Based Vectors

Immunogenicity was assessed using the model antigen TIPeGFP in order to determine whether comparable immunogenicity to AdC63 and AdC68 could be obtained in mice using an AdY25-based vector.

Balb/c mice (4/group) were immunised intramuscularly with 109 infectious units (ifu) of each of the following viral vectors, all expressing the TIPeGFP antigen:
v. AdCh63;
vi. ΔE1 ΔE3 AdCh68; and
vii. ChAdOX1.

After 14 days post-prime, spleen immunogenicity against a strong CD8+ epitope (Pb9) was assessed by IFN-γ ELISpot

The IFN-γ spleen ELISpot responses are shown in FIG. 4. Responses elicited by ChAdOX1 were robust and comparable to those seen using AdCh63 and the AdCh68-based vector. These data support the continued development of AdY25-based vectors for clinical application.

### Example 5: Effect of E4 Modification on Immunogenicity of AdY25-Based Vectors

The impact of two different E4 modifications on the immunogenicity of AdY25-based vectors was assessed using the following constructs:
(i) AdY25 E4 wildtype (“E4 wt”)
(ii) AdY25 E4AdHu5Orf6 (“E4Orf6”); and
(iii) AdY25 E4AdHu5Orf4/6/7(“E4Orf4/6/7”).

Balb/c mice (4/group) were immunised intramuscularly with either 106 ifu or 108 ifu of each vector. Responses to Pb9 and PI 5 epitopes were assayed two weeks post immunisation. Titers calculated once again on GFP to remove the effect of hexon production rates on vaccine titer.

The effect of E4 modification on IFN-7 spleen ELISpot responses is shown in FIGS. 5A and 5B. The data indicate that E4 modification has no effect on vector immunogenicity. Therefore, such modifications can be used to enhance the rate of production of the viral vectors, without having a negative impact on the immunogenicity of the vectors.

### Example 6: Prevalence of Vector-Neutralising Antibodies

The prevalence of vector neutralising antibodies in human sera from the UK and The Gambia against AdY25-based vectors and AdCh63-based vectors was assessed.

HEK293 cells were infected with Y25Ad5E4Orf6-SEAP or AdCh63-SEAP (SEAP=Secreted Placental Alkaline Phosphatase). Recombinant adenoviruses were incubated with five serial dilutions of serum in FBS-DMEM before infection. The final serum dilutions were 1: 18, 1:72, 1:288, 1:1 152, 1:4608; each serum sample was tested in duplicate. Supernatants were collected and assayed for SEAP concentration using CSPD (Tropix) according to the manufacturer's instructions. Luminescence intensity was measured using a Varioskan flash luminometer (Thermo Scientific).

Neutralization titers were defined as the serum dilution required to reduce SEAP concentration by 50% compared to wells infected with virus alone. Neutralization titer was calculated by linear interpolation of adjacent values.

As shown in FIG. 6, the seroprevalence of neutralising antibodies against Y25Ad5E4Orf6 was surprisingly found to be much lower than that for AdCh63 in both the UK and The Gambia.

### Example 7: Humoral Immunogenicity of AdY25-Based Vectors

Balb/c mice (6/group) were immunised with 108 infectious units of either of the following vectors, both expressing TIPeGFP:
viii. ΔE1 ΔE3 AdCh68; or
ix. ChAdOX1.

After 56 days post prime, mice were boosted with 106 pfu MVA-TIPeGFP. Serum was collected 50 days post-prime and 10 days post-boost to compare pre- and post-boost anti-GFP antibody responses. Responses were measured by endpoint ELISA. Statistical analyses were performed by one way ANOVA.

As shown in FIG. 7, humoral immunogenicity of the AdY25-based vector ChAdOX1 is superior to current chimpanzee adenovirus vector AdCh68, indicating an enhanced antibody response elicited by the AdY25-based vector in comparison to the AdCh68-based vector.

### Example 8: Induction of Immune Response Against Mycobacterium tuberculosis

A transgene encoding the Mycobacterium tuberculosis protein Ag85A was inserted into the E1 locus of ChAdOX1 under control of the human cytomegalovirus immediate early promoter, using the BAC technology as described in Example 1. The nucleotide sequence of the transgene (SEQ ID NO. 42) encodes residues 1 to 323 of the antigen, encoded by a sequence optimised to human codon usage (nucleotides 103 to 1071), fused at the N-terminus to tPA (the signal peptide from human tissue plasminogen activator)(nucleotides 1 to 102) and at the C-terminus to a PK tag (nucleotides 1072 to 1 104). The amino acid sequence of the Ag85A antigen is provided in SEQ ID NO.43.

The BAC clone was transfected into HEK293 cells and the virus vector was amplified, purified and titred using the anti-hexon immunostaining assay described in Example 3.

The immunogenicity of the vector was assessed in Balb/c mice immunized with varying doses, expressed in infectious units, of the vaccine, administered intramuscularly. After 14 days cellular immune responses to Ag85A were determined by IFN-7 ELIspot assay using splenocytes stimulated with synthetic peptides corresponding to the known immunodominant CD8+(pi 1; WYDQSGLSV (SEQ ID NO. 44)) and CD4+ T cell (pi 5; TFLTSELPGWLQANRHVKPT (SEQ ID NO. 45)) H-2d restricted epitopes in Ag85A.

The results are shown in FIGS. 8A and 8B. These results indicate that the ChAdOX1 vector is capable of inducing immune responses against Mycobacterium tuberculosis. The magnitude of these responses is similar to that induced by vectors based on other adenoviruses.

### Example 9: Induction of Immune Response Against Influenza A

A transgene encoding the nucleoprotein (NP) and matrix protein 1 (M1) of influenza A virus was inserted into the E1 locus of ChAdOX1 under control of the human cytomegalovirus major immediate early promoter, using the BAC technology as described in Example 1. The nucleotide sequence of the transgene (SEQ ID NO. 46) encodes the influenza A nucleoprotein (nucleotides 1 to 1494) fused to the matrix protein 1 (nucleotides 1516 to 2274) and separated by a linker (nucleotides 1495 to 1515). The amino acid sequence of the NPM1 fusion protein is provided in SEQ ID NO. 47.

The BAC clone was transfected into HEK293 cells and the virus vector was amplified, purified and titred using the anti-hexon immunostaining assay described in Example 3. A similar vector based on human adenovirus type 5 (HAdV-5) was similarly generated and titred for comparative purposes.

The immunogenicity of the vector was assessed in Balb/c mice immunized with varying doses, expressed in infectious units, of the vaccine, administered intramuscularly. After 14 days cellular immune responses to NP were determined by IFN-7 ELIspot assay using splenocytes stimulated with synthetic peptides corresponding to the known immunodominant CD8+ T cell H-2d restricted epitope in NP ((TYQRTRALV) (SEQ ID NO. 48)).

The results are shown in FIG. 9. These results indicate that the ChAdOX1 vector is capable of inducing immune responses against influenza A virus and that, at the doses tested, these are similar to those induced by a HAdV-5 vector.

The ChAdOX1-NPM1 vaccine has recently been produced for human clinical trials according to current good manufacturing practice at the University of Oxford Clinical Biomanufacturing Facility. This indicates the suitability of the vector for deployment as a medical product.

### REFERENCES

1. Buchbinder et al, Lancet, Vol 372, November 2008
2. Farina et al, J. Virol, December 2001, p11603-11613
3. Dudareva et al, Vaccine 27, 2009, 3501-3504
4. R. Wigand et al, Intervirology, Vol 30; 1 1989
5. Roy et al, Hum. Gen. Ther., 2004, 15:519-530
6. Warming et al. Nuc. Acid. Res, 2005, Vol 33; 4
7. http://www.invitrogen.com/gateway
8. Havenga et al, J.G.V., 2006, 87, 2135-214
9. Wanning, S. et al. Nucleic Acids Res, 2005, February 24; 33(4): e36

1-22. (canceled)

23. A method of treating a disease in a subject using immunotherapy, said immunotherapy comprising: administering to the subject an adenovirus comprising a capsid comprising: one or more capsid proteins from chimpanzee adenovirus AdY25 which encapsidates a nucleic acid molecule comprising an exogeneous nucleotide sequence of interest encoding an antigen specific to the disease operably linked to expression control sequences and an adenoviral packaging signal sequence; wherein administering the adenovirus to the subject elicits or boosts an immune response in the subject to treat the disease.

24. The method of claim 23, wherein the disease is a cancer or an infectious disease.

25. The method of claim 24, wherein the cancer is small-cell lung cancer, neuroblastoma, melanoma, osteosarcoma, or breast cancer.

26. The method of claim 24, wherein the infectious disease is selected from the group consisting of Tuberculosis and other mycobacterial infections, malaria, influenza, HIV/AIDS, Hepatitis C, Cytomegalovirus infection, Human papilloma virus infection, adenoviral infection, leishmaniasis, streptococcus spp., staphylococcus spp., meningococcus spp., infection, rift valley fever, foot and mouth disease and chikungunya virus infection.

27. The method of claim 23, wherein the subject is a human subject.

28. The method of claim 23, wherein the adenovirus comprises an AdY25 genome that lacks a functional E1 locus.

29. The method of claim 23, wherein the adenovirus comprises an AdY25 genome that lacks an E3 locus.

30. The method of claim 23, wherein the adenovirus comprises an AdY25 genome with an E4 locus wherein at least one of the E4 open reading frames (ORF) is heterologous.

31. The method of claim 30, wherein the entire E4 locus is heterologous.

32. The method of claim 31, wherein the E4 ORF is E4Orf6 from AdHu5.

33. The method of claim 30, wherein the adenovirus has an E4 locus that comprises E4Orf1, E4Orf2 and E4Orf3 from AdY25 and E4Orf4, E4Orf6 and E4Orf6/7 from AdHu5.

34. The method of claim 23, wherein the antigen is a self-antigen.

35. The method of claim 34, wherein the self-antigen is GD2 or ErbB2.

36. The method of claim 25, wherein the exogenous nucleotide sequence of interest further encodes a tumor associated antigen (TAA), preferably wherein the TAA is from a pathogen involved in cancer immunopathogenesis.

37. The method of claim 36, wherein the antigen from a pathogen involved in cancer immunopathogenesis is from human papilloma virus (HPV), hepatitis B virus (HBV) or Epstein Barr virus (EBY).

38. The method of claim 23, wherein the one or more capsid proteins from the AdY25 are selected from the group consisting of:
(a) an AdY25 hexon protein comprising the amino acid sequence of SEQ ID NO. 2;
(b) an AdY25 penton protein comprising the amino acid sequence of SEQ ID NO. 3; and
(c) an AdY25 fibre protein comprising the amino acid sequence of SEQ ID NO. 4.

39. The method of claim 23, wherein the administering comprises oral, parenteral, mucosal, rectal, or transdermal administration of the adenovirus to the subject.

40. The method of claim 25, wherein the administering is direct to the cancer.


SEQUENCE LISTING (49 sequences)

SEQ ID NO: 1 - DNA, 36711 bp, Adenovirus AdY25
  misc_feature 1..36711: note = source = /mol_type="DNA" /organism="Chimpanzee adenovirus AdY25"
  source 1..36711: mol_type = genomic DNA
ccatcatcaataatatacctcaaactttttgtgcgcgttaatatgcaaatgaggcgtttgaatttgggaagggaggaaggtgattggccgagagaagggcgaccgttaggggcggggcgagtgacgttttgatgacgtgaccgcgaggaggagccagtttgcaagttctcgtgggaaaagtgacgtcaaacgaggtgtggtttgaacacggaaatactcaattttcccgcgctctctgacaggaaatgaggtgtttctaggcggatgcaagtgaaaacgggccattttcgcgcgaaaactgaatgaggaagtgaaaatctgagtaatttcgcgtttatgacagggaggagtatttgccgagggccgagtagactttgaccgattacgtgggggtttcgattaccgtgtttttcacctaaatttccgcgtacggtgtcaaagtccggtgtttttacgtaggtgtcagctgatcgccagggtatttaaacctgcgctctccagtcaagaggccactcttgagtgccagcgagaagagttttctcctccgcgccgcgagtcagatctacactttgaaagatgaggcacctgagagacctgcccgatgagaaaatcatcatcgcttccgggaacgagattctggaactggtggtaaatgccatgatgggcgacgaccctccggagccccccaccccatttgaggcaccttcgctacacgatttgtatgatctggaggtggatgtgcccgaggacgaccccaacgaggaggcggtaaatgatttatttagcgatgccgcgctgctagctgccgaggaggcttcgagccctagctcagacagcgactcttcactgcatacccctagacccggcagaggtgagaaaaagatccccgagcttaaaggggaagagatggacttgcgctgctatgaggaatgcttgcccccgagcgatgatgaggacgagcaggcgatccagaacgcagcgagccagggaatgcaagccgccagcgagagttttgcgctggactgcccgcctctgcccggacacggctgtaagtcttgtgaatttcatcgcttgaatactggagataaagctgtgttatgtgcactttgctatatgagagcttacaaccattgtgtttacagtaagtgtgattaagttgaactttagagggaggcagagagcagggtgactgggcgatgactggtttatttatgtatatatgttctttatataggtcccgtctctgacgcagatgatgagacccccactacagagtccacttcgtcacccccagaaattggcacatctccacctgagaatattgttagaccagttcctgttagagccactgggaggagagcagctgtggaatgtttggatgacttgctacaggctggggatgaacctttggacttgtgtacccggaaacgccccaggcactaagtgccacacatgtgtgtttacttgaggtgatgtcagtatttatagggtgtggagtgcaataaaaaatgtgttgactttaagtgcgtggtttatgactcaggggtggggactgtgggtatataagcaggtgcagacctgtgtggttagctcagagcggcatggagatttggacgatcttggaagatcttcacaagactagacagctgctagagaacgcctcgaacggagtctctcacctgtggagattctgcttcggtggcgacctagctaagctagtctatagggccaaacaggattatagcgaacaatttgaggttattttgagagagtgtccgggtctttttgacgctcttaatttgggtcatcagactcactttaaccagaggattgtaagagcccttgattttactactcccggcagatccactgcggcagtagccttttttgcttttcttcttgacaaatggagtcaagaaacccatttcagcagggattaccagctggatttcttagcagtagctttgtggagaacatggaaatcccagcgcctgaatgcaatctcaggctacttgccggtacagccactagacactctgaagatcctgaatctccaggagagtcccagggcacgccaacgtcgccggcagcagcagcggcagcaggaggaggatcaagaagagaacccgagagccggcctggaccctccggcggaggaggagtagctgacctgtttcctgaactgcgccgggtgctgactaggtcttcgagtggtcgggagagggggattaagcgggagaggcatgatgagactaatcacagaactgaactgactgtgggtctgatgagccgcaagcgtccagaaacagtgtggtggcatgaggtgcagtcgactggcacagatgaggtgtcagtgatgcatgagaggttttccctagaacaagtcaagacttgttggttagagcctgaggatgattgggaggtagccatcaggaattatgccaagctggctctgaggccagacaagaagtacaagattactaagctgataaatatcagaaatgcctgctacatctcagggaatggggctgaagtggagatctgtcttcaggaaagggtggctttcagatgctgcatgatgaatatgtacccgggagtggtgggcatggatggggtcacctttatgaacatgaggttcaggggagatgggtataatggcacggtctttatggccaataccaagctgacagttcatggctgctccttctttgggtttaataacacctgcattgaggcctggggtcaggttggtgtgaggggctgtagtttttcagccaactggatgggggtcgtgggcaggaccaagagtatgctgtccgtgaagaaatgcttgttcgagaggtgccacctgggggtgatgagcgagggcgaagccagaatccgccactgcgcctctaccgagacgggctgttttgtgctgtgcaagggcaatgctaagatcaagcataatatgatctgtggagcctcggacgagcgcggctaccagatgctgacctgcgccggtgggaacagccatatgctggccaccgtgcatgtggcctcccatgcccgcaagccctggcccgagttcgagcacaatgtcatgaccaggtgcaatatgcatctggggtcccgccgaggcatgttcatgccctatcagtgcaacctgaattatgtgaaggtgctgctggagcccgatgccatgtccagagtgagcctgacgggggtgtttgacatgaatgtggaggtgtggaagattctgagatatgatgaatccaagaccaggtgccgagcctgcgagtgcggagggaagcatgccaggttccagcccgtgtgtgtggaggtgacggaggacctgcgacccgatcatttggtgttgtcctgcaccgggacggagttcggttccagcggggaagaatctgactagagtgagtagtgttctggggcgggggaggacctgcatgagggccagaatgactgaaatctgtgcttttctgtgtgttgcagcatcatgagcggaagcggctcctttgagggaggggtattcagcccttatctgacggggcgtctcccctcctgggcgggagtgcgtcagaatgtgatgggatccacggtggacggccggcccgtgcagcccgcgaactcttcaaccctgacctatgcaaccctgagctcttcgtcggtggacgcagctgccgccgcagctgctgcatccgccgccagcgccgtgcgcggaatggccatgggcgccggctactacggcactctggtggccaactcgagttccaccaataatcccgccagcctgaacgaggagaagctgctgctgctgatggcccagcttgaggccttgacccagcgcctgggcgagctgacccagcaggtggctcagctgcaggagcagacgcgggccgcggttgccacggtgaaatccaaataaaaaatgaatcaataaataaacggagacggttgttgattttaacacagagtctgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcgtggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcccttgtgtccgcccaggttttccatgcactcatccatgatgatggcaatgggcccgtgggcggcggcctgggcaaagacgtttcgggggtcggacacatcatagttgtggtcctgggtgaggtcatcataggccattttaatgaatttggggcggagggtgccggactgggggacaaaggtaccctcgatcccgggggcgtagttcccctcacagatctgcatctcccaggctttgagctcagagggggggatcatgtccacctgcggggcgataaagaacacggtttccggggcgggggagatgagctgggccgaaagcaagttccggagcagctgggacttgccgcagccggtggggccgtaaatgaccccgatgaccggctgcaggtggtagttgagggagagacagctgccgtcctcccggaggaggggggccacctcgttcatcatctcgcgcacgtgcatgttctcgcgcaccagttccgccaggaggcgctctccccccagagataggagctcctggagcgaggcgaagtttttcagcggcttgagtccgtcggccatgggcattttggagagggtctgttgcaagagttccaagcggtcccagagctcggtgatgtgctctacggcatctcgatccagcagacctcctcgtttcgcgggttgggacgactgcgggagtagggcaccagacgatgggcgtccagcgcagccagggtccggtccttccagggccgcagcgtccgcgtcagggtggtctccgtcacggtgaaggggtgcgcgccgggctgggcgcttgcgagggtgcgcttcaggctcatccggctggtcgaaaaccgctcccgatcggcgccctgcgcgtcggccaggtagcaattgaccatgagttcgtagttgagcgcctcggccgcgtggcctttggcgcggagcttacctttggaagtctgcccgcaggcgggacagaggagggacttgagggcgtagagcttgggggcgaggaagacggaatcgggggcgtaggcgtccgcgccgcagtgggcgcagacggtctcgcactccacgagccaggtgaggtcgggctggtcggggtcaaaaaccagtttcccgccgttctttttgatgcgtttcttacctttggtctccatgagctcgtgtccccgctgggtgacaaagaggctgtccgtgtccccgtagaccgactttatgggccggtcctcgagcggtgtgccgcggtcctcctcgtagaggaaccccgcccactccgagacgaaagcccgggtccaggccagcacgaaggaggccacgtgggacgggtagcggtcgttgtccaccagcgggtccactttttccagggtatgcaaacacatgtccccctcgtccacatccaggaaggtgattggcttgtaagtgtaggccacgtgaccgggggtcccggccgggggggtataaaagggggcgggcccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccagcggagatgcctttcaagagcccctcgtccatctggtcagaaaagacgatttttttgttgtcgagcttggtggcgaaggagccgtagagggcgttggaaaggagcttggcgatggagcgcatggtctggtttttttccttgtcggcgcgctccttggccgcgatgttgagctgcacgtactcgcgcgccacgcacttccattcggggaagacggtggtcatctcgtcgggcacgattctgacctgccaacctcgattatgcagggtgatgaggtccacactggtggccacctcgccgcgcaggggctcgttggtccagcagaggcggccgcccttgcgcgagcagaaggggggcagagggtccagcatgacctcgtcgggggggtcggcatcgatggtgaagatgccgggcaggagatcggggtcgaagtagctgatggaagtggccagatcgtccagggaagcttgccattcgcgcacggccagcgcgcgctcgtagggactgaggggcgtgccccagggcatggggtgggtgagcgcggaggcgtacatgccgcagatgtcgtagacgtagaggggctcctcgaggatgccgatgtaggtggggtagcagcgccccccgcggatgctggcgcgcacgtagtcatacagctcgtgcgagggcgcgaggagccccgggcccaggttggtgcgactgggcttttcggcgcggtagacgatctggcgaaagatggcatgcgagttggaggagatggtgggcctttggaagatgttgaagtgggcgtgggggaggccgaccgagtcgcggatgaagtgggcgtaggagtcttgcagtttggcgacgagctcggcggtgacgaggacgtccagagcgcagtagtcgagggtctcctggatgatgtcatacttgagctggcccttttgtttccacagctcgcggttgagaaggaactcttcgcggtccttccagtactcttcgagggggaacccgtcctgatctgcacggtaagagcctagcatgtagaactggttgacggccttgtaggcgcagcagcccttctccacggggagggcgtaggcctgggcggccttgcgcagggaggtgtgcgtgagggcgaaggtgtccctgaccatgaccttgaggaactggtgcttgaaatcgatatcgtcgcagcccccctgctcccagagctggaagtccgtgcgcttcttgtaggcggggttgggcaaagcgaaagtaacatcgttgaaaaggatcttgcccgcgcggggcataaagttgcgagtgatgcggaaaggctggggcacctcggcccggttgttgatgacctgggcggcgagcacgatctcgtcgaaaccgttgatgttgtggcccacgatgtagagttccacgaatcgcgggcggcccttgacgtggggcagcttcttgagctcctcgtaggtgagctcgtcggggtcgctgagaccgtgctgctcgagcgcccagtcggcgagatgggggttggcgcggaggaaggaagtccagagatccacggccagggcggtttgcagacggtcccggtactgacggaactgctgcccgacggccattttttcgggggtgacgcagtagaaggtgcgggggtccccgtgccagcggtcccatttgagctggagggcgagatcgagggcgagctcgacgaggcggtcgtcccctgagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccgttggtggcgacgtcgatggcttgcagggtcccgtgcccctggggtgtgaccaccgtcccccgtttcttcttgggcggctggggcgacgggggcggtgcctcttccatggttagaagcggcggcgaggacgcgcgccgggcggcagaggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcctgaaggtctccgcgaccggcgcgctccacggtggccgcgaggtcgttggagatgcggcccatgagctgcgagaaggcgttcatgcccgcctcgttccagacgcggctgtagaccacgacgccctcgggatcgcgggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaagcgttccatggcctcgtaaaagtccacggcgaagttgaaaaactgggagttgcgcgccgagacggtcaactcctcctccagaagacggatgagctcggcgatggtggcgcgcacctcgcgctcgaaggcccccgggagttcctccacttcctcctcttcttcctcctccactaacatctcttctacttcctcctcaggcggtggtggtggcgggggagggggcctgcgtcgccggcggcgcacgggcagacggtcgatgaagcgctcgatggtctcgccgcgccggcgtcgcatggtctcggtgacggcgcgcccgtcctcgcggggccgcagcgtgaagacgccgccgcgcatctccaggtggccgggggggtccccgttgggcagggagagggcgctgacgatgcatcttatcaattgccccgtagggactccgcgcaaggacctgagcgtctcgagatccacgggatctgaaaaccgttgaacgaaggcttcgagccagtcgcagtcgcaaggtaggctgagcacggtttcttctgccgggtcatgttggggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccaggtccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcctgctggatctgggtgagggtggtctggaagtcgtcaaagtcgacgaagcggtggtaggctccggtgttgatggtgtaggagcagttggccatgacggaccagttgacggtctggtggcccggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtactggcactcccgtctcgacccaagcctgcaccaaccctccaggatacggaggcgggtcgttttgcaactttttttggaggccggaaatgaaactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagaccccatagccagccgacttctccagttacggagcgagcccctcttttgttttgtttgtttttgccagatgcatcccgtactgcggcagatgcgcccccaccaccctccaccgcaacaacagccccctcctccacagccggcgcttctgcccccgccccagcagcagcagcaacttccagccacgaccgccgcggccgccgtgagcggggctggacagacttctcagtatgatcacctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaggcgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagtctgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgcgcggtgagcgccagcaggcggcgcgagctgagcgaccaggagctgatgcacagcctgcagcgggccctgaccggggccgggaccgagggggagagctactttgacatgggcgcggacctgcactggcagcccagccgccgggccttggaggcggcaggcggtcccccctacatagaagaggtggacgatgaggtggacgaggagggcgagtacctggaagactgatggcgcgaccgtatttttgctagatgcaacaacagccacctcctgatcccgcgatgcgggcggcgctgcagagccagccgtccggcattaactcctcggacgattggacccaggccatgcaacgcatcatggcgctgacgacccgcaaccccgaagcctttagacagcagccccaggccaaccggctctcggccatcctggaggccgtggtgccctcgcgctccaaccccacgcacgagaaggtcctggccatcgtgaacgcgctggtggagaacaaggccatccgcggcgacgaggccggcctggtgtacaacgcgctgctggagcgcgtggcccgctacaacagcaccaacgtgcagaccaacctggaccgcatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggcgttcaagaacttgcagggcctgtggggcgtgcaggccccggtcggggaccgcgcgacggtgtcgagcctgctgacgccgaactcgcgcctgctgctgctgctggtggcccccttcacggacagcggcagcatcaaccgcaactcgtacctgggctacctgattaacctgtaccgcgaggccatcggccaggcgcacgtggacgagcagacctaccaggagatcacccacgtgagccgcgccctgggccaggacgacccgggcaatctggaagccaccctgaactttttgctgaccaaccggtcgcagaagatcccgccccagtacacgctcagcgccgaggaggagcgcatcctgcgatacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctgaatccccactggctcccgccgccggggttctacacgggcgagtacgacatgcccgaccccaatgacgggttcctgtgggacgatgtggacagcagcgtgttctccccccgaccgggtgctaacgagcgccccttgtggaagaaggaaggcagcgaccgacgcccgtcctcggcgctgtccggccgcgagggtgctgccgcggcggtgcccgaggccgccagtcctttcccgagcttgcccttctcgctgaacagtattcgcagcagcgagctgggcaggatcacgcgcccgcgcttgctgggcgaggaggagtacttgaatgactcgctgttgagacccgagcgggagaagaacttccccaataacgggatagagagcctggtggacaagatgagccgctggaagacgtatgcgcaggagcacagggacgatccgtcgcagggggccacgagccggggcagcgccgcccgtaaacgccggtggcacgacaggcagcggggactgatgtgggacgatgaggattccgccgacgacagcagcgtgttggacttgggtgggagtggtaacccgttcgctcacctgcgcccccgcatcgggcgcatgatgtaagagaaaccgaaaataaatgatactcaccaaggccatggcgaccagcgtgcgttcgtttcttctctgttgttgtatctagtatgatgaggcgtgcgtacccggagggtcctcctccctcgtacgagagcgtgatgcagcaggcgatggcggcggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggtcagctgaaaaccatcatgcacaccaacatgcccaacgtgaacgagttcatgtacagcaacaagttcaaggcgcgggtgatggtctcccgcaagacccccaacggggtgacagtgacagatggtagtcaggatatcttggagtatgaatgggtggagtttgagctgcccgaaggcaacttctcggtgaccatgaccatcgacctgatgaacaacgccatcatcgacaattacttggcggtggggcggcagaacggggtcctggagagcgatatcggcgtgaagttcgacactaggaacttcaggctgggctgggaccccgtgaccgagctggtcatgcccggggtgtacaccaacgaggccttccaccccgatattgtcttgctgcccggctgcggggtggacttcaccgagagccgcctcagcaacctgctgggcattcgcaagaggcagcccttccaggagggcttccagatcatgtacgaggatctggaggggggcaacatccccgcgctcctggatgtcgacgcctatgagaaaagcaaggaggagagcgccgccgcggcgactgcagctgtagccaccgcctctaccgaggtcaggggcgataattttgccagccctgcagcagtggcagcggccgaggcggctgaaaccgaaagtaagatagtcattcagccggtggagaaggatagcaaggacaggagctacaacgtgctgccggacaagataaacaccgcctaccgcagctggtacctggcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgctcacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaaaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcggtcgcgcaccaccgtcgacgacgtgatcgaccaggtggtggccgacgcgcgcaactacacccccgccgccgcgcccgtctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcttcaggcgccagcgccggcaggacccggagacgcgcggccacggcggcggcagcggccatcgccagcatgtcccgcccgcggcgagggaacgtgtactgggtgcgcgacgccgccaccggtgtgcgcgtgcccgtgcgcacccgcccccctcgcacttgaagatgttcacttcgcgatgttgatgtgtcccagcggcgaggaggatgtccaagcgcaaattcaaggaagagatgctccaggtcatcgcgcctgagatctacggccccgcggtggtgaaggaggaaagaaagccccgcaaaatcaagcgggtcaaaaaggacaaaaaggaagaagatgacgatctggtggagtttgtgcgcgagttcgccccccggcggcgcgtgcagtggcgcgggcggaaagtgcacccggtgctgagacccggcaccaccgtggtcttcacgcccggcgagcgctccggcagcgcttccaagcgctcctacgacgaggtgtacggggacgaggacatcctcgagcaggcggccgagcgcctgggcgagtttgcttacggcaagcgcagccgccccgccctgaaggaagaggcggtgtccatcccgctggaccacggcaaccccacgccgagcctcaagcccgtgaccctgcagcaggtgctgccgagcgcagcgccgcgccgggggttcaagcgcgagggcgaggatctgtaccccaccatgcagctgatggtgcccaagcgccagaagctggaagacgtgctggagaccatgaaggtggacccggacgtgcagcccgaggtcaaggtgcggcccatcaagcaggtggccccgggcctgggcgtgcagaccgtggacatcaagatccccacggagcccatggaaacgcagaccgagcccatgatcaagcccagcaccagcaccatggaggtgcagacggatccctggatgccatcggctcctagccgaagaccccggcgcaagtacggcgcggccagcctgctgatgcccaactacgcgctgcatccttccatcatccccacgccgggctaccgcggcacgcgcttctaccgcggtcatacaaccagccgccgccgcaagaccaccacccgccgccgccgtcgccgcacagccgctgcatctacccctgccgccctggtgcggagagtgtaccgccgcggccgcgcgcctctgaccctaccgcgcgcgcgctaccacccgagcatcgccatttaaactttcgcctgctttgcagatggccctcacatgccgcctccgcgttcccattacgggctaccgaggaagaaaaccgcgccgtagaaggctggcggggaacgggatgcgtcgccaccaccatcggcggcggcgcgccatcagcaagcggttggggggaggcttcctgcccgcgctgatccccatcatcgccgcggcgatcggggcgatccccggcattgcttccgtggcggtgcaggcctctcagcgccactgagacacttggaaaacatcttgtaataaaccaatggactctgacgctcctggtcctgtgatgtgttttcgtagacagatggaagacatcaatttttcgtccctggctccgcgacacggcacgcggccgttcatgggcacctggagcgacatcggcaccagccaactgaacgggggcgccttcaattggagcagtctctggagcgggcttaagaatttcgggtccacgcttaaaacctatggcagcaaggcgtggaacagcaccacagggcaggcgctgagggataagctgaaagagcagaacttccagcagaaggtggtcgatgggctcgcctcgggcatcaacggggtggtggacctggccaaccaggccgtgcagcggcagatcaacagccgcctggacccggtgccgcccgccggctccgtggagatgccgcaggtggaggaggagctgcctcccctggacaagcggggcgagaagcgaccccgccccgacgcggaggagacgctgctgacgcacacggacgagccgcccccgtacgaggaggcggtgaaactgggtctgcccaccacgcggcccatcgcgcccctggccaccggggtgctgaaacccgaaagtaataagcccgcgaccctggacttgcctcctcccgcttcccgcccctctacagtggctaagcccctgccgccggtggccgtggcccgcgcgcgacccgggggctccgcccgccctcatgcgaactggcagagcactctgaacagcatcgtgggtctgggagtgcagagtgtgaagcgccgccgctgctattaaacctaccgtagcgcttaacttgcttgtctgtgtgtgtatgtattatgtcgccgctgtccgccagaaggaggagtgaagaggcgcgtcgccgagttgcaagatggccaccccatcgatgctgccccagtgggcgtacatgcacatcgccggacaggacgcttcggagtacctgagtccgggtctggtgcagttcgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccctagcttcaaaccctactccggcaccgcctacaacagcctggctcccaagggagcgcccaattccagccagtgggagcaaaaaaaggcaggcaatggtgacactatggaaacacacacatttggtgtggccccaatgggcggtgagaatattacaatcgacggattacaaattggaactgacgctacagctgatcaggataaaccaatttatgctgacaaaacattccagcctgaacctcaagtaggagaagaaaattggcaagaaactgaaagcttttatggcggtagggctcttaaaaaagacacaagcatgaaaccttgctatggctcctatgctagacccaccaatgtaaagggaggtcaagctaaacttaaagttggagctgatggagttcctaccaaagaatttgacatagacctggctttctttgatactcccggtggcacagtgaatggacaagatgagtataaagcagacattgtcatgtataccgaaaacacgtatctggaaactccagacacgcatgtggtatacaaaccaggcaaggatgatgcaagttctgaaattaacctggttcagcagtccatgcccaatagacccaactatattgggttcagagacaactttattgggctcatgtattacaacagtactggcaatatgggggtgctggctggtcaggcctcacagctgaatgctgtggtcgacttgcaagacagaaacaccgagctgtcataccagctcttgcttgactctttgggtgacagaacccggtatttcagtatgtggaatcaggcggtggacagttatgatcctgatgtgcgcattattgaaaaccatggtgtggaagacgaacttcccaactattgcttccccctggatgggtctggcactaatgccgcttaccaaggtgtgaaagtaaaaaatggtaacgatggtgatgttgagagcgaatgggaaaatgatgatactgtcgcagctcgaaatcaattatgcaagggcaacatttttgccatggaaattaacctccaagccaacctgtggagaagtttcctctactcgaacgtggccctgtacctgcccgactcttacaagtacacgccagccaacatcaccctgcccaccaacaccaacacttatgattacatgaacgggagagtggtgcctccctcgctggtggacgcctacatcaacatcggggcgcgctggtcgctggaccccatggacaacgtcaatcccttcaaccaccaccgcaacgcgggcctgcgctaccgctccatgctcctgggcaacgggcgctacgtgcccttccacatccaggtgccccagaaatttttcgccatcaagagcctcctgctcctgcccgggtcctacacctacgagtggaacttccgcaaggacgtcaacatgatcctgcagagctccctcggcaacgacctgcgcacggacggggcctccatctccttcaccagcatcaacctctacgccaccttcttccccatggcgcacaacacggcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgcctcaagaccaaggagacgccctcgctgggctccgggttcgacccctacttcgtctactcgggctccatcccctacctcgacggcaccttctacctcaaccacaccttcaagaaggtctccatcaccttcgactcctccgtcagctggcccggcaacgaccggctcctgacgcccaacgagttcgaaatcaagcgcaccgtcgacggcgagggatacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtggtggacgaggtcaactacaaggactaccaggccgtcaccctggcctaccagcacaacaactcgggcttcgtcggctacctcgcgcccaccatgcgccagggccagccctaccccgccaactacccgtacccgctcatcggcaagagcgccgtcaccagcgtcacccagaaaaagttcctctgcgacagggtcatgtggcgcatccccttctccagcaacttcatgtccatgggcgcgctcaccgacctcggccagaacatgctctatgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacccccttctcggccggtaacgccaccacctaaattgctacttgcatgatggctgagcccacaggctccggcgagcaggagctcagggccatcatccgcgacctgggctgcgggccctacttcctgggcaccttcgataagcgcttcccgggattcatggccccgcacaagctggcctgcgccatcgtcaacacggccggccgcgagaccgggggcgagcactggctggccttcgcctggaacccgcgctcgaacacctgctacctcttcgaccccttcgggttctcggacgagcgcctcaagcagatctaccagttcgagtacgagggcctgctgcgccgtagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaactcccactccgcctactttcgctcccaccgcgcgcgcatcgagaaggccaccgccttcgaccgcatgaacaatcaagacatgtaaaccgtgtgtgtatgtttaaaatatcttttaataaacagcactttaatgttacacatgcatctgagatgattttattttagaaatcgaaagggttctgccgggtctcggcatggcccgcgggcagggacacgttgcggaactggtacttggccagccacttgaactcggggatcagcagtttgggcagcggggtgtcggggaaggagtcggtccacagcttccgcgtcagctgcagggcgcccagcaggtcgggcgcggagatcttgaaatcgcagttgggacccgcgttctgcgcgcgagagttgcggtacacggggttgcagcactggaacaccatcagggccgggtgcttcacgctcgccagcaccgccgcgtcggtgatgctctccacgtcgaggtcctcggcgttggccatcccgaagggggtcatcttgcaggtctgccttcccatggtgggcacgcacccgggcttgtggttgcaatcgcagtgcagggggatcagcatcatctgggcctggtcggcgttcatccccgggtacatggccttcatgaaagcctccaattgcctgaacgcctgctgggccttggctccctcggtgaagaagaccccgcaggacttgctagagaactggttggtggcacagccggcatcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcatgtgctccttctggatcatggtggtcccgtgcaggcaccgcagtttgccctcggcctcggtgcacccgtgcagccacagcgcgcacccggtgcactcccagttcttgtgggcgatctgggaatgcgcgtgcacgaacccttgcaggaagcggcccatcatggtcgtcagggtcttgttgctagtgaaggtcaacgggatgccgcggtgctcctcgttgatgtacaggtggcagatgcggcggtacacctcgccctgctcgggcatcagttggaagttggctttcaggtcggtctccacgcggtagcggtccatcagcatagtcatgatttccatgcccttctcccaggccgagacgatgggcaggctcatagggttcttcaccatcatcttagcactagcagccgcggccagggggtcgctctcatccagggtctcaaagctccgcttgccgtccttctcggtgatccgcaccggggggtagctgaagcccacggccgccagctcctcctcggcctgtctttcgtcctcgctgtcctggctgacgtcctgcatgaccacatgcttggtcttgcggggtttcttcttgggcggcagtggcggcggagatgcttgtggcgagggggagcgcgagttctcgctcaccactactatctcttcctcttcttggtccgaggccacgcggcggtaggtatgtctcttcgggggcagaggcggaggcgacgggctctcgccgccgcgacttggcggatggctggcagagccccttccgcgttcgggggtgcgctcccggcggcgctctgactgacttcctccgcggccggccattgtgttctcctagggaggaacaacaagcatggagactcagccatcgccaacctcgccatctgcccccaccgccggcgacgagaagcagcagcagcagaatgaaagcttaaccgccccgccgcccagccccgcctccgacgcagccgcggtcccagacatgcaagagatggaggaatccatcgagattgacctgggctatgtgacgcccgcggagcatgaggaggagctggcagtgcgctttcaatcgtcaagccaggaagataaagaacagccagagcaggaagcagagaacgagcagagtcaggctgggctcgagcatggcgactacctccacctgagcggggaggaggacgcgctcatcaagcatctggcccggcaggccaccatcgtcaaggacgcgctgctcgaccgcaccgaggtgcccctcagcgtggaggagctcagccgcgcctacgagctcaacctcttctcgccgcgcgtgccccccaagcgccagcccaacggcacctgcgagcccaacccccgcctcaacttctacccggtcttcgcggtgcccgaggccctggccacctaccacatctttttcaagaaccaaaagatccccgtctcctgccgcgccaaccgcacccgcgccgacgccctcttcaacctgggtcccggcgcccgcctacctgatatcgcctccttggaagaggttcccaagatcttcgagggtctgggcagcgacgagactcgggccgcgaacgctctgcaaggagaaggaggaggagagcatgagcaccacagcgccctggtcgagttggaaggcgacaacgcgcggctggcggtgctcaaacgcacggtcgagctgacccatttcgcctacccggctctgaacctgcccccgaaagtcatgagcgcggtcatggaccaggtgctcatcaagcgcgcgtcgcccatctccgaggacgagggcatgcaagactccgaggagggcaagcccgtggtcagcgacgagcagctggcccggtggctgggtcctaatgctacccctcaaagtttggaagagcggcgcaagctcatgatggccgtggtcctggtgaccgtggagctggagtgcctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcttgcacgagaaccgcctggggcagaacgtgctgcacaccaccctgcgcggggaggcccgccgcgactacatccgcgactgcgtctacctctacctctgccacacctggcagacgggcatgggcgtgtggcagcagtgtctggaggagcagaacctgaaagagctctgcaagctcctgcaaaagaacctcaagggtctgtggaccgggttcgacgagcggaccaccgcctcggacctggccgacctcatcttccccgagcgcctcaggctgacgctgcgcaacggcctgcccgactttatgagccaaagcatgttgcaaaactttcgctctttcatcctcgaacgctccggaatcctgcccgccacctgctccgcgctgccctcggacttcgtgccgctgaccttccgcgagtgccccccgccgctgtggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaagggcccagcgagggcgagggagccaaggggggtctgaaactcaccccggggctgtggacctcggcctacttgcgcaagttcgtgcccgaggattaccatcccttcgagatcaggttctacgaggaccaatcccagccgcccaaggccgagctgtcggcctgcgtcatcacccagggggcgatcctggcccaattgcaagccatccagaaatcccgccaagaattcttgctgaaaaagggccgcggggtctacctcgacccccagaccggtgaggagctcaaccccggcttcccccaggatgccccgaggaaacaagaagctgaaagtggagctgccgcccgtggaggatttggaggaagactgggagaacagcagtcaggcagaggagatggaggaagactgggacagcactcaggcagaggaggacagcctgcaagacagtctggaggaagacgaggaggaggcagaggaggaggtggaagaagcagccgccgccagaccgtcgtcctcggcgggggagaaagcaagcagcacggataccatctccgctccgggtcggggtcccgctcggccccacagtagatgggacgagaccgggcgattcccgaaccccaccacccagaccggtaagaaggagcggcagggatacaagtcctggcgggggcacaaaaacgccatcgtctcctgcttgcaggcctgcgggggcaacatctccttcacccggcgctacctgctcttccaccgcggggtgaacttcccccgcaacatcttgcattactaccgtcacctccacagcccctactacttccaagaagaggcagcagcagcagaaaaagaccagaaaaccagctagaaaatccacagcggcggcagcggcaggtggactgaggatcgcggcgaacgagccggcgcagacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccagtcgcagaaaaaggcgggaattacgtcacctgtgcccttcgccctagccgcctccacccagcaccgccatgagcaaagagattcccacgccttacatgtggagctaccagccccagatgggcctggccgccggcgccgcccaggactactccacccgcatgaattggctcagcgccgggcccgcgatgatctcacgggtgaatgacatccgcgcccaccgaaaccagatactcctagaacagtcagcgctcaccgccacgccccgcaatcacctcaatccgcgtaattggcccgccgccctggtgtaccaggaaattccccagcccacgaccgtactacttccgcgagacgcccaggccgaagtccagctgactaactcaggtgtccagctggcgggcggcgccaccctgtgtcgtcaccgccccgctcagggtataaagcggctggtgatccggggcagaggcacacagctcaacgacgaggtggtgagctcttcgctgggtctgcgacctgacggagtcttccaactcgccggatcggggagatcttccttcacgcctcgtcaggcggtcctgactttggagagttcgtcctcgcagccccgctcgggcggcatcggcactctccagttcgtggaggagttcactccctcggtctacttcaaccccttctccggctcccccggccactacccggacgagttcatcccgaactttgacgccatcagcgagtcggtggacggctacgattgaatgtcccatggtggcgcggctgacctagctcggcttcgacacctggaccactgccgccgctttcgctgcttcgctcgggacctcgccgagttcacctactttgagctgcccgaggagcatcctcagggcccggcccacggagtgcggatcgtcgtcgaagggggcctagactcccacctgcttcggatcttcagccagcgcccgatcctggtcgagcgccaacagggcaacaccctcctgaccctctactgcatctgcgaccaccccggcctgcatgaaagtctttgttgtctgctgtgtactgagtataataaaagctgagatcagcgactactccggactcaactgtggtgtttctgcatccatcaatcggtctctgaccttcaccgggaacgagaccgagctccaggtccagtgtaagccccacaagaagtacctcacctggctgtaccagggctccccgatcgccgttgttaaccactgcgacgacgacggagtcctgctgaacggccccgccaaccttactttttccacccgcagaagcaagctactgctcttccgacccttcctccccggcacctatcagtgcatctcgggaccctgccatcacaccttccacctgatcccgaataccacctcttccccagcaccgctccccactaacaaccaaactaaccaccaccaacgctaccgacgcgacctcgtttctgaatctaataccacccacaccggaggtgagctccgaggtcgcaaaccctctgggatttattacggcccctgggaggtggtggggttaatagctttaggcttagtggcgggtgggcttttggctctctgctacctatacctcccttgcttttcctacttagtggtgctttgttgctggtttaagaaatggggaagatcaccctagtgtgcggtgtgctggtgacggtggtgctttcgattctgggagggggaagcgcggctgtagtgacggagaagaaggccgatccctgcttgactttcaaccccgataaatgccggctgagttttcagcccgatggcaatcggtgcgcggtgttgatcaagtgcggatgggaatgcgagagcgtgttggtccagtataaaaacaagacctggaacaatactctcgcgtccacatggcagcccggggaccccgagtggtacaccgtctctgtccctggtgctgacggctccctccgcacggtgaacaacactttcatttttgagcacatgtgcgagaccgccatgttcatgagcaagcagtacggtatgtggcccccacgtaaagagaatatcgtggtcttctccatcgcttacagcgcgtgcacggtgctaatcaccgcgatcgtgtgcctgagcattcacatgctcatcgctattcgccccagaaataatgccgagaaagagaaacagccataacacacttttcacatacctttttcagaccatggcctctgttacaatccttatttattttttgggacttgtgggcactagcagcacttttcagcatataaacaaaactgtttatgctggttcaaattctgtgttagctggacatcagtcataccagaaagtttcatggtactggtatgataaaaatcaaacacccgttacactctgcaagggtccacaacagcccgtaaaccgtagtgggattttttttagctgtaatcataataatatcacactactttcaattacaaagcactatgctggaacttactatggaaccaatttcaatatcaaacatgacacttactatagtgtcagagtattggatccaactacccctagaacaactacaaagcccaccacaactaagaagcccactacacctaagaagcctaccacgcccaaaaccactaagacaactactaagaccactaccacagagccaaccacaaccagcacccacacttgctataactacacacacacacacacactgagctgacctcacaggcaactactgaaaatggttttgccctgttacaaaagggggaaaacagtagcagcagtcctctgcctaccacccccagtgaggaaatacctaaatccatggttggcattatcgctgctgtagtggtgtgtatgctgattatcatcttgtgcatgatgtactatgcctgctactacagaaaacacaggctgaacaacaagctggaccccctactgaatgttgatttttaattttttagaaccatgaagatcctaagccttttttgtttttctataattattacctctgctatttgtaactcagtggataaggacgttactgtcaccactggctctaattatacactgaaaggacctccctcaggtatgctttcgtggtattgctattttggaactgatgtttcacaaactgaattgtgtaattttcaaaaaggcaaaacccaaaatcctaaaattcataactatcaatgcaatggtactgatttagtactgttcaatatcacgaaaacatatgctggaagttattactgcccgggagataatgttgacaatatgattttttacgaattacaagtagttgatcccactactccagcaccacccaccacaactaccaaggcacatagcacagacacacaggaaaccactccagaggcagaagtagcagagttagcaaagcagattcatgaagattcctttgttgccaatacccccacacaccccggaccgcaatgtccagggccattagtcagcggcattgtcggtgtgctttgcgggttagcagttataatcatctgcatgttcatttttgcttgctgctacagaaggcttcaccgacaaaaatcagacccactgctgaacctctatgtttaatttttgattttccagagccatgaaggcacttagcactttagtttttttgaccttgattggcattgtttttaatagtaaaattaccagggttagctttctcaaacatgttaatgttactgaaggaaataatatcacactagtaggtgtagaaggtgctcaaaacaccacctggacaaaataccatctcgggtggaaagatatttgcacctggaatgtcacttatttttgcataggagttaatcttaccattgttaatgctaatcaatctcagaatggattaattaaagggcagagcgtgagtgttaccagtgatgggtactatacccagcataatttcaactacaacattactgttataccactgccaacacctagcccacctagcactactcagaccacacaaacaactcacactacacagagctccacaactaccatgcagaccactcagacaaccacatacactacttcccctcagcccaccaccactacagcagaggcgagtagctcacccaccatcaaagtggcatttttaatgctggccccatctagcagtcccactgctagtaccaatgagcagactactgaatttttgtccactattcagagcagcaccacagctacctcgagtgccttctctagcaccgccaatctcacctcgctttcctctatgccaatcagtaatgctactacctcccccgctcctcttcccactcctctgaagcaatccgagtccagcacgcagctgcagatcaccctgctcattgtgatcggggtggtcatcctggcagtgctgctctactttatcttctgccgtcgcatccccaacgcaaagccggcctacaagcccattgttatcgggacgccggagccgcttcaggtggagggaggtctaaggaatcttctcttctcttttacagtatggtgatttgaactatgattcctagacatttcattatcacttctctaatctgtgtgctccaagtctgtgccaccctcgctctcgtggctaacgcgagtccagactgcattggagcgttcgcctcctacgtgctctttgccttcatcacctgcatctgctgctgtagcatagtctgcctgcttatcaccttcttccagttcgttgactgggtctttgtgcgcatcgcctacctgcgccaccacccccagtaccgcgaccagagagtggcgcaactgttgagactcatctgatgataagcatgcgggctctgctactacttctcgcgcttctgctagctcccctcgccgcccccctatccctcaaatcccccacccagtcccctgaagaggttcgaaaatgtaaattccaagaaccctggaaattcctttcatgctacaaactcaaatcagaaatgcaccccagctggatcatgatcgttggaatcgtgaacatccttgcctgtaccctcttctcctttgtgatttacccccgctttgactttgggtggaacgcacccgaggcgctctggctcccgcctgatcccgacacaccaccacagcagcagcagcaaaatcaggcacaggcacacgcaccaccacagcctaggccacaatacatgcccatcttaaactatgaggccgaggcacagcgagccatgcttcctgctattagttacttcaatctaaccggcggagatgactgaccccatggccaacaacaccgtcaacgacctcctggacatggacggccgcgcctcggagcagcgactcgcccaactccgcatccgccagcagcaggagagagccgtcaaggagctgcaggatgcggtggccatccaccagtgcaagagaggcatcttctgcctggtgaagcaggccaagatctccttcgaggtcacgtccaccgaccatcgcctctcctacgagctcctgcagcagcgccagaagttcacctgcctggtcggagtcaaccccatcgtcatcacccagcagtctggcgataccaagggttgcatccactgctcctgcgactcccccgagtgcgttcacaccctgatcaagaccctctgcggcctccgcgacctcctccccatgaactaatcaactaaccccctacccctttaccctccagtaaaaataaagattaaaaatgattgaattgatcaataaagaatcacttacttgaaatctgaaaccaggtctctgtccatgttttctgtcagcagcacttcactcccctcttcccaactctggtactgcaggccccggcgggctgcaaacttcctccacactctgaaggggatgtcaaattcctcctgtccctcaatcttcatttttatcttctatcagatgtccaaaaagcgcgcgcgggtggatgatggcttcgaccccgtgtacccctacgatgcagacaacgcaccgactgtgcccttcatcaaccctcccttcgtctcttcagatggattccaagaaaagcccctgggggtgttgtccctgcgactggccgaccccgtcaccaccaagaatggggctgtcaccctcaagctgggggagggggtggacctcgacgactcgggaaaactcatctccaaaaatgccaccaaggccactgcccctctcagtatttccaacggcaccatttcccttaacatggctgcccctttttacaacaacaatggaacgttaagtctcaatgtttctacaccattagcagtatttcccacttttaacactttaggtatcagtcttggaaacggtcttcaaacttctaataagttgctgactgtacagttaactcatcctcttacattcagctcaaatagcatcacagtaaaaacagacaaaggactctatattaattctagtggaaacagagggcttgaggctaacataagcctaaaaagaggactgatttttgatggtaatgctattgcaacataccttggaagtggtttagactatggatcctatgatagcgatgggaaaacaagacccatcatcaccaaaattggagcaggtttgaattttgatgctaataatgccatggctgtgaagctaggcacaggtttaagttttgactctgccggtgccttaacagctggaaacaaagaggatgacaagctaacactttggactacacctgacccaagccctaattgtcaattactttcagacagagatgccaaatttaccctatgtcttacaaaatgcggtagtcaaatactaggcactgttgcagtagctgctgttactgtaggttcagcactaaatccaattaatgacacagtaaaaagcgccatagtattccttagatttgactctgacggtgtgctcatgtcaaactcatcaatggtaggtgattactggaactttagggaaggacagaccacccaaagtgtggcctatacaaatgctgtgggattcatgcccaatctaggtgcatatcctaaaacccaaagcaaaacaccaaaaaatagtatagtaagtcaggtatatttaaatggagaaactactatgccaatgacactgacaataactttcaatggcactgatgaaaaagacacaacacctgtgagcacttactccatgacttttacatggcagtggactggagactataaggacaagaatattacctttgctaccaactcctttactttctcctacatggcccaagaataaaccctgcatgccaaccccattgttcccaccactatggaaaactctgaagcagaaaaaaataaagttcaagtgttttattgattcaacagttttcacagaattcgagtagttattttccctcctccctcccaactcatggaatacaccaccctctccccacgcacagccttaaacatctgaatgccattggtaatggacatggttttggtctccacattccacacagtttcagagcgagccagtctcgggtcggtcagggagatgaaaccctccgggcactcctgcatctgcacctcaaagttcagtagctgagggctgtcctcggtggtcgggatcacagttatctggaagaagagcggtgagagtcataatccgcgaacgggatcgggcggttgtggcgcatcaggccccgcagcagtcgctgtctgcgccgctccgtcaagctgctgctcaaggggtctgggtccagggactccctgcgcatgatgccgatggccctgagcatcagtcgcctggtgcggcgggcgcagcagcggatgcggatctcactcaggtcggagcagtacgtgcagcacagcactaccaagttgttcaacagtccatagttcaacgtgctccagccaaaactcatctgtggaactatgctgcccacatgtccatcgtaccagatcctgatgtaaatcaggtggcgccccctccagaacacactgcccatgtacatgatctccttgggcatgtgcaggttcaccacctcccggtaccacatcacccgctggttgaacatgcagccctggataatcctgcggaaccagatggccagcaccgccccgcccgccatgcagcgcagggaccccgggtcctggcaatggcagtggagcacccaccgctcacggccgtggattaactgggagctgaacaagtctatgttggcacagcacaggcacacgctcatgcatgtcttcagcactctcagttcctcgggggtcaggaccatgtcccagggcacggggaactcttgcaggacagtgaacccggcagaacagggcagccctcgcacacaacttacattgtgcatggacagggtatcgcaatcaggcagcaccggatgatcctccaccagagaagcgcgggtctcggtctcctcacagcgaggtaagggggccggcggttggtacggatgatggcgggatgacgctaatcgtgttctggatcgtgtcatgatggagctgtttcctgacattttcgtacttcacgaagcagaacctggtacgggcactgcacaccgctcgccggcgacggtctcggcgcttcgagcgctcggtgttgaagttatagaacagccactccctcagagcgtgcagtatctcctgagcctcttgggtgatgaaaatcccatccgctctgatggctctgatcacatcggccacggtggaatgggccagacccagccagatgatgcaattttgttgggtttcggtgacggagggagagggaagaacaggaagaaccatgattaactttattccaaacggtctcggagcacttcaaaatgcaggtcccggaggtggcacctctcgcccccactgtgttggtggaaaataacagccaggtcaaaggtgacacggttctcgagatgttccacggtggcttccagcaaagcctccacgcgcacatccagaaacaagaggacagcgaaagcgggagcgttttctaattcctcaatcatcatattacactcctgcaccatccccagataattttcatttttccagccttgaatgattcgtattagttcctgaggtaaatccaagccagccatgataaaaagctcgcgcagagcgccctccaccggcattcttaagcacaccctcataattccaagagattctgctcctggttcacctgcagcagattaacaatgggaatatcaaaatctctgccgcgatccctaagctcctccctcaacaataactgtatgtaatctttcatatcatctccgaaatttttagccatagggccgccaggaataagagcagggcaagccacattacagataaagcgaagtcctccccagtgagcattgccaaatgtaagattgaaataagcatgctggctagaccctgtgatatcttccagataactggacagaaaatcaggcaagcaatttttaagaaaatcaacaaaagaaaagtcgtccaggtgcaggtttagagcctcaggaacaacgatggaataagtgcaaggagtgcgttccagcatggttagtgtttttttggtgatctgtagaacaaaaaataaacatgcaatattaaaccatgctagcctggcgaacaggtgggtaaatcactctttccagcaccaggcaggctacggggtctccggcgcgaccctcgtagaagctgtcgccatgattgaaaagcatcaccgagagaccttcccggtggccggcatggatgattcgagaagaagcatacactccgggaacattggcatccgtgagtgaaaaaaagcgacctataaagcctcggggcactacaatgctcaatctcaattccagcaaagccaccccatgcggatggagcacaaaattggcaggtgcgtaaaaaatgtaattactcccctcctgcacaggcagcaaagcccccgctccctccagaaacacatacaaagcctcagcgtccatagcttaccgagcacggcaggcgcaagagtcagagaaaaggctgagctctaacctgactgcccgctcctgtgctcaatatatagccctaacctacactgacgtaaaggccaaagtctaaaaatacccgccaaaatgacacacacgcccagcacacgcccagaaaccggtgacacactcaaaaaaatacgtgcgcttcctcaaacgcccaaaccggcgtcatttccgggttcccacgctacgtcaccgctcagcgactttcaaattccgtcgaccgttaaaaacgtcactcgccccgcccctaacggtcgcccttctctcggccaatcaccttcctcccttcccaaattcaaacgcctcatttgcatattaacgcgcacaaaaagtttgaggtatatatttgaatgatg

SEQ ID NO: 2 - protein, 942 aa, synthetic construct
  REGION 1..942: note = SOURCE - /mol_type="protein" /organism="Chimpanzee adenovirus AdY25"
MATPSMLPQWAYMHIAGQDASEYLSPGLVQFARATDTYFSLGNKFRNPTVAPTHDVTTDRSQRLTLRFVPVDREDNTYSYKVRYTLAVGDNRVLDMASTYFDIRGVLDRGPSFKPYSGTAYNSLAPKGAPNSSQWEQKKAGNGDTMETHTFGVAPMGGENITIDGLQIGTDATADQDKPIYADKTFQPEPQVGEENWQETESFYGGRALKKDTSMKPCYGSYARPTNVKGGQAKLKVGADGVPTKEFDIDLAFFDTPGGTVNGQDEYKADIVMYTENTYLETPDTHVVYKPGKDDASSEINLVQQSMPNRPNYIGFRDNFIGLMYYNSTGNMGVLAGQASQLNAVVDLQDRNTELSYQLLLDSLGDRTRYFSMWNQAVDSYDPDVRIIENHGVEDELPNYCFPLDGSGTNAAYQGVKVKNGNDGDVESEWENDDTVAARNQLCKGNIFAMEINLQANLWRSFLYSNVALYLPDSYKYTPANITLPTNTNTYDYMNGRVVPPSLVDAYINIGARWSLDPMDNVNPFNHHRNAGLRYRSMLLGNGRYVPFHIQVPQKFFAIKSLLLLPGSYTYEWNFRKDVNMILQSSLGNDLRTDGASISFTSINLYATFFPMAHNTASTLEAMLRNDTNDQSFNDYLSAANMLYPIPANATNVPISIPSRNWAAFRGWSFTRLKTKETPSLGSGFDPYFVYSGSIPYLDGTFYLNHTFKKVSITFDSSVSWPGNDRLLTPNEFEIKRTVDGEGYNVAQCNMTKDWFLVQMLAHYNIGYQGFYVPEGYKDRMYSFFRNFQPMSRQVVDEVNYKDYQAVTLAYQHNNSGFVGYLAPTMRQGQPYPANYPYPLIGKSAVTSVTQKKFLCDRVMWRIPFSSNFMSMGALTDLGQNMLYANSAHALDMNFEVDPMDESTLLYVVFEVFDVVRVHQPHRGVIEAVYLRTPFSAGNATT

SEQ ID NO: 3 - protein, 532 aa, synthetic construct
  REGION 1..532: note = SOURCE - /mol_type="protein" /organism="Chimpanzee adenovirus AdY25"
MMRRAYPEGPPPSYESVMQQAMAAAAAMQPPLEAPYVPPRYLAPTEGRNSIRYSELAPLYDTTRLYLVDNKSADIASLNYQNDHSNFLTTVVQNNDFTPTEASTQTINFDERSRWGGQLKTIMHTNMPNVNEFMYSNKFKARVMVSRKTPNGVTVTDGSQDILEYEWVEFELPEGNFSVTMTIDLMNNAIIDNYLAVGRQNGVLESDIGVKFDTRNFRLGWDPVTELVMPGVYTNEAFHPDIVLLPGCGVDFTESRLSNLLGIRKRQPFQEGFQIMYEDLEGGNIPALLDVDAYEKSKEESAAAATAAVATASTEVRGDNFASPAAVAAAEAAETESKIVIQPVEKDSKDRSYNVLPDKINTAYRSWYLAYNYGDPEKGVRSWTLLTTSDVTCGVEQVYWSLPDMMQDPVTFRSTRQVSNYPVVGAELLPVYSKSFFNEQAVYSQQLRAFTSLTHVFNRFPENQILVRPPAPTITTVSENVPALTDHGTLPLRSSIRGVQRVTVTDARRRTCPYVYKALGIVAPRVLSSRTF

SEQ ID NO: 4 - protein, 443 aa, synthetic construct
  REGION 1..443: note = SOURCE - /mol_type="protein" /organism="Chimpanzee adenovirus AdY25"
MSKKRARVDDGFDPVYPYDADNAPTVPFINPPFVSSDGFQEKPLGVLSLRLADPVTTKNGAVTLKLGEGVDLDDSGKLISKNATKATAPLSISNGTISLNMAAPFYNNNGTLSLNVSTPLAVFPTFNTLGISLGNGLQTSNKLLTVQLTHPLTFSSNSITVKTDKGLYINSSGNRGLEANISLKRGLIFDGNAIATYLGSGLDYGSYDSDGKTRPIITKIGAGLNFDANNAMAVKLGTGLSFDSAGALTAGNKEDDKLTLWTTPDPSPNCQLLSDRDAKFTLCLTKCGSQILGTVAVAAVTVGSALNPINDTVKSAIVFLRFDSDGVLMSNSSMVGDYWNFREGQTTQSVAYTNAVGFMPNLGAYPKTQSKTPKNSIVSQVYLNGETTMPMTLTITFNGTDEKDTTPVSTYSMTFTWQWTGDYKDKNITFATNSFTFSYMAQE

SEQ ID NO: 5 - protein, 257 aa, synthetic construct
  REGION 1..257: note = SOURCE - /mol_type="protein" /organism="Chimpanzee adenovirus AdY25"
MRHLRDLPDEKIIIASGNEILELVVNAMMGDDPPEPPTPFEAPSLHDLYDLEVDVPEDDPNEEAVNDLFSDAALLAAEEASSPSSDSDSSLHTPRPGRGEKKIPELKGEEMDLRCYEECLPPSDDEDEQAIQNAASQGMQAASESFALDCPPLPGHGCKSCEFHRLNTGDKAVLCALCYMRAYNHCVYSPVSDADDETPTTESTSSPPEIGTSPPENIVRPVPVRATGRRAAVECLDDLLQAGDEPLDLCTRKRPRH

SEQ ID NO: 6 - protein, 187 aa, synthetic construct
  REGION 1..187: note = SOURCE - /mol_type="protein" /organism="Chimpanzee adenovirus AdY25"
MEIWTILEDLHKTRQLLENASNGVSHLWRFCFGGDLAKLVYRAKQDYSEQFEVILRECPGLFDALNLGHQTHFNQRIVRALDFTTPGRSTAAVAFFAFLLDKWSQETHFSRDYQLDFLAVALWRTWKSQRLNAISGYLPVQPLDTLKILNLQESPRARQRRRQQQRQQEEDQEENPRAGLDPPAEEE

SEQ ID NO: 7 - protein, 499 aa, synthetic construct
  REGION 1..499: note = SOURCE - /mol_type="protein" /organism="Chimpanzee adenovirus AdY25"
MESRNPFQQGLPAGFLSSSFVENMEIPAPECNLRLLAGTATRHSEDPESPGESQGTPTSPAAAAAAGGGSRREPESRPGPSGGGGVADLFPELRRVLTRSSSGRERGIKRERHDETNHRTELTVGLMSRKRPETVWWHEVQSTGTDEVSVMHERFSLEQVKTCWLEPEDDWEVAIRNYAKLALRPDKKYKITKLINIRNACYISGNGAEVEICLQERVAFRCCMMNMYPGVVGMDGVTFMNMRFRGDGYNGTVFMANTKLTVHGCSFFGFNNTCIEAWGQVGVRGCSFSANWMGVVGRTKSMLSVKKCLFERCHLGVMSEGEARIRHCASTETGCFVLCKGNAKIKHNMICGASDERGYQMLTCAGGNSHMLATVHVASHARKPWPEFEHNVMTRCNMHLGSRRGMFMPYQCNLNYVKVLLEPDAMSRVSLTGVFDMNVEVWKILRYDESKTRCRACECGGKHARFQPVCVEVTEDLRPDHLVLSCTGTEFGSSGEESD

SEQ ID NO: 8 - protein, 142 aa, synthetic construct
  REGION 1..142: note = SOURCE - /mol_type="protein" /organism="Chimpanzee adenovirus AdY25"
MSGSGSFEGGVFSPYLTGRLPSWAGVRQNVMGSTVDGRPVQPANSSTLTYATLSSSSVDAAAAAAAASAASAVRGMAMGAGYYGTLVANSSSTNNPASLNEEKLLLLMAQLEALTQRLGELTQQVAQLQEQTRAAVATVKSK

SEQ ID NO: 9 - protein, 449 aa, synthetic construct
  REGION 1..449: note = SOURCE - /mol_type="protein" /organism="Chimpanzee adenovirus AdY25"
METKGRRRSGAVFDQPDEPEAHPRKRPARRAPLHRDGDHPDADAAALEGPDPGCAGRPSSGALLPQSSQPAKRGGLLDRDAVEHITELWDRLELLQQTLSKMPMADGLKPLKNFASLQELLSLGGERLLAELVRENMHVREMMNEVAPLLREDGSCLSLNYHLQPVIGVIYGPTGCGKSQLLRNLLSAQLISPAPETVFFIAPQVDMIPPSELKAWEMQICEGNYAPGIEGTFVPQSGTLRPKFIKMAYDDLTQDHNYDVSDPRNVFAQAAAHGPIAIIMDECMENLGGHKGVAKFFHAFPSKLHDKFPKCTGYTVLVVLHNMNPRRDLGGNIANLKIQAKMHLISPRMHPSQLNRFVNTYTKGLPVAISLLLKDIVQHHALRPCYDWVIYNTTPEHEALQWSYLHPRDGLMPMYLNIQAHLYRVLEKIHRVLNDRDRWSRAYRARKIK

SEQ ID NO: 10 - protein, 1196 aa, synthetic construct
  REGION 1..1196: note = SOURCE - /mol_type="protein" /organism="Chimpanzee adenovirus AdY25"
MALVQTHGSRGLHPEASDPGRQPSRRRSRQSSPGAVPEPTRARRRRAPAAPASGPRAASAARRASSPPLLTMEEAPPPSPQPPKKKRGTVVTPQGHGTLQAIDVATNGAVEIKYHLDLPRALEKLLQVNRAPPLPTDLTPQRLRTLDSSGLRALVLALRPARAEVWTCLPRGLVSMTTIEAEEGQADHHDVVQHEMQAPRLHFPLKFLVKGTQVQLVQHVHPVQRCEHCGRLYKHKHECSARRRHFYFHHINSHSSNWWQEIQFFPIGSHPRTERLFLTYDVETYTWMGSFGKQLVPFMLVMKLSGDDRLVELALDLALQLKWDRWHGDPRTFYCVTPEKMAVGQQFRQYRDRLQTALAVDLWTSFLRANPHLADWALEQHGLSDPDELTYEELKKLPHVKGRPRFVELYIVGHNINGFDEIVLAAQVINNRAEVPQPFRITRNFMPRAGKILFNDVTFALPNPAYKKRTDFQLWEQGGCDDIDFKHQFLKVMVRDTFALTHTSLRKAAQAYALPVEKGCCAYKAVNQFYMLGSYRADQDGFPLEEYWKDREEFLLNRELWKQKGQLKYDIIQETLDYCALDVLVTAELVAKLQDSYAHFIRDSVGLPHAHFNIFQRPTISSNSHAIFRQIVYRAEKPSRTNLGPGLLAPSHELYDYVRASIRGGRCYPTYIGILEEPLYVYDICGMYASALTHPMPWGTPLSPYERALAVREWQASLDDLATSISYFDPDLLPGIFTIDADPPDEVMLDPLPPFCSRKGGRLCWTNEPLRGEVATSVDLITLHNRGWQVRIVPDEMTTVFPEWKCVAREYVQLNIAAKERADKEKNQTMRSIAKLLSNALYGSFATKLDNKKIVFSDQMDEGLLKGISAGTVNIKSSSFLETDNLSAEVMPAFEREYLPQQLALLDSDPEDSEDEQGPAPFYTPPAGTPGHVAYTYKPITFLDVDEGDMCLHTLEKVDPLVDNDRYPSHVASFVLAWTRAFVSEWAGFLYEEDRGTPLEDRPIKSVYGDTDSLFVTQRGHELMETKGKKRIKKNGGKLVFDPDQPDLTWLVECETVCAHCGADAYAPDSVFLAPKLYALKSLLCPACGQTSKGKLRAKGHAAEALNYELMVNCYLADAQGADRERFSTSRMSLKRTLASAQPGAHPFTVTETTLTRTLRPWKDRTLAALDAHRLVPYSRSRPNPRNEEVCWIEMP

SEQ ID NO: 11 - protein, 646 aa, synthetic construct
  REGION 1..646: note = SOURCE - /mol_type="protein" /organism="Chimpanzee adenovirus AdY25"
MALSIHDCARLTGQTVPTMNYFLPLRNIWNRVREFPRASTTAAGITWMSRYIYGYHRTMLEDLAPGAPATERWPLYRQPPPHFLIGYQYLVRTCNDYIFDTRAYSRLKYHELVRPGHQTVNWSVMANCSYTINTGAYHRFVDFDDFQTTLTQIQQAILAERVVADLALVQPQRGFGLTRMHGRAGEEEVPVERLMQDYYKDLARCQDHAWGMADRLRIQQAGPKDLVLLATIRRLRTAYFNFITSSIARPAPQHDPAEETVLSLPCDCDWLEAFVQRFSDPVDLETLRSLRGVPTGQLIRCIVSALSLPNGDPPGHLEMRGGVFTLRPREDGRAVTETMRRRRGETIERFIDRLPVRRRRRRPPPPPPPPPEEEVEEMLVEEEEEEEVEELPGAFEREVRATIAELIRLLEEELTVSARNSQFFNFAVDFYEAMERLEALGDVSEMPLRRWIMYFFVTEHIATTLNYLYQRLCNYAVFTRHVELNLAQVVMRARDPEGVVVYSRVWNEAGMNAFSQLMGRISNDLAATVERAGRGDLQEEEIEQFMTEIAYQDNSGDVQEILRQAAVNDTEIDSVELSFRFKLTGPVAFTQRRQIQDVNRRVVAHASLLRAQYQNLPARGADVPLPPLPPGPEPPLPPGARPRRRF

SEQ ID NO: 12 - protein, 396 aa, synthetic construct
  REGION 1..396: note = SOURCE - /mol_type="protein" /organism="Chimpanzee adenovirus AdY25"
MHPVLRQMRPHHPPPQQQPPPPQPALLPPPQQQQQLPATTAAAAVSGAGQTSQYDHLALEEGEGLARLGASSPERHPRVQMKRDAREAYVPKQNLFRDRSGEEPEEMRAARFHAGRELRRGLDRKRVLRDEDFEADELTGISPARAHVAAANLVTAYEQTVKEESNFQKSFNNHVRTLIAREEVTLGLMHLWDLLEAIVQNPTSKPLTAQLFLVVQHSRDNEAFREALLNITEPEGRWLLDLVNILQSIVVQERGLPLSEKLAAINFSVLSLGKYYARKIYKTPYVPIDKEVKIDGFYMRMTLKVLTLSDDLGVYRNDRMHRAVSASRRRELSDQELMHSLQRALTGAGTEGESYFDMGADLHWQPSRRALEAAGGPPYIEEVDDEVDEEGEYLED

SEQ ID NO: 13 - protein, 589 aa, synthetic construct
  REGION 1..589: note = SOURCE - /mol_type="protein" /organism="Chimpanzee adenovirus AdY25"
MQQQPPPDPAMRAALQSQPSGINSSDDWTQAMQRIMALTTRNPEAFRQQPQANRLSAILEAVVPSRSNPTHEKVLAIVNALVENKAIRGDEAGLVYNALLERVARYNSTNVQTNLDRMVTDVREAVAQRERFHRESNLGSMVALNAFLSTQPANVPRGQEDYTNFISALRLMVTEVPQSEVYQSGPDYFFQTSRQGLQTVNLSQAFKNLQGLWGVQAPVGDRATVSSLLTPNSRLLLLLVAPFTDSGSINRNSYLGYLINLYREAIGQAHVDEQTYQEITHVSRALGQDDPGNLEATLNFLLTNRSQKIPPQYTLSAEEERILRYVQQSVGLFLMQEGATPSAALDMTARNMEPSMYASNRPFINKLMDYLHRAAAMNSDYFTNAILNPHWLPPPGFYTGEYDMPDPNDGFLWDDVDSSVFSPRPGANERPLWKKEGSDRRPSSALSGREGAAAAVPEAASPFPSLPFSLNSIRSSELGRITRPRLLGEEEYLNDSLLRPEREKNFPNNGIESLVDKMSRWKTYAQEHRDDPSQGATSRGSAARKRRWHDRQRGLMWDDEDSADDSSVLDLGGSGNPFAHLRPRIGRMM

SEQ ID NO: 14 - protein, 193 aa, synthetic construct
  REGION 1..193: note = SOURCE - /mol_type="protein" /organism="Chimpanzee adenovirus AdY25"
MSILISPSNNTGWGLRAPSKMYGGARQRSTQHPVRVRGHFRAPWGALKGRVRSRTTVDDVIDQVVADARNYTPAAAPVSTVDAVIDSVVADARRYARAKSRRRRIARRHRSTPAMRAARALLRRARRTGRRAMLRAARRAASGASAGRTRRRAATAAAAAIASMSRPRRGNVYWVRDAATGVRVPVRTRPPRT

SEQ ID NO: 15 - protein, 340 aa, synthetic construct
  REGION 1..340: note = SOURCE - /mol_type="protein" /organism="Chimpanzee adenovirus AdY25"
MSKRKFKEEMLQVIAPEIYGPAVVKEERKPRKIKRVKKDKKEEDDDLVEFVREFAPRRRVQWRGRKVHPVLRPGTTVVFTPGERSGSASKRSYDEVYGDEDILEQAAERLGEFAYGKRSRPALKEEAVSIPLDHGNPTPSLKPVTLQQVLPSAAPRRGFKREGEDLYPTMQLMVPKRQKLEDVLETMKVDPDVQPEVKVRPIKQVAPGLGVQTVDIKIPTEPMETQTEPMIKPSTSTMEVQTDPWMPSAPSRRPRRKYGAASLLMPNYALHPSIIPTPGYRGTRFYRGHTTSRRRKTTTRRRRRRTAAASTPAALVRRVYRRGRAPLTLPRARYHPSIAI

SEQ ID NO: 16 - protein, 77 aa, synthetic construct
  REGION 1..77: note = SOURCE - /mol_type="protein" /organism="Chimpanzee adenovirus AdY25"
MALTCRLRVPITGYRGRKPRRRRLAGNGMRRHHHRRRRAISKRLGGGFLPALIPIIAAAIGAIPGIASVAVQASQRH

SEQ ID NO: 17 - protein, 243 aa, synthetic construct
  REGION 1..243: note = SOURCE - /mol_type="protein" /organism="Chimpanzee adenovirus AdY25"
MEDINFSSLAPRHGTRPFMGTWSDIGTSQLNGGAFNWSSLWSGLKNFGSTLKTYGSKAWNSTTGQALRDKLKEQNFQQKVVDGLASGINGVVDLANQAVQRQINSRLDPVPPAGSVEMPQVEEELPPLDKRGEKRPRPDAEETLLTHTDEPPPYEEAVKLGLPTTRPIAPLATGVLKPESNKPATLDLPPPASRPSTVAKPLPPVAVARARPGGSARPHANWQSTLNSIVGLGVQSVKRRRCY

SEQ ID NO: 18 - protein, 209 aa, synthetic construct
  REGION 1..209: note = SOURCE - /mol_type="protein" /organism="Chimpanzee adenovirus AdY25"
MAEPTGSGEQELRAIIRDLGCGPYFLGTFDKRFPGFMAPHKLACAIVNTAGRETGGEHWLAFAWNPRSNTCYLFDPFGFSDERLKQIYQFEYEGLLRRSALATEDRCVTLEKSTQTVQGPRSAACGLFCCMFLHAFVHWPDRPMDKNPTMNLLTGVPNGMLQSPQVEPTLRRNQEALYRFLNSHSAYFRSHRARIEKATAFDRMNNQDM

SEQ ID NO: 19 - protein, 512 aa, synthetic construct
  REGION 1..512: note = SOURCE - /mol_type="protein" /organism="Chimpanzee adenovirus AdY25"
MAGRGGSQSERRRERTPERGRGSASHPPSRGGESPSPPPLPPKRHTYRRVASDQEEEEIVVVSENSRSPSPQASPPPLPPKKKPRKTKHVVMQDVSQDSEDERQAEEELAAVGFSYPPVRITEKDGKRSFETLDESDPLAAAASAKMMVKNPMSLPIVSAWEKGMEIMTMLMDRYRVETDLKANFQLMPEQGEVYRRICHLYINEEHRGIPLTFTSNKTLTTMMGRFLQGFVHAHSQIAHKNWECTGCALWLHGCTEAEGKLRCLHGTTMIQKEHMIEMDVASENGQRALKENPDRAKITQNRWGRSVVQLANNDARCCVHDAGCATNQFSSKSCGVFFTEGAKAQQAFRQLEAFMKAMYPGMNADQAQMMLIPLHCDCNHKPGCVPTMGRQTCKMTPFGMANAEDLDVESITDAAVLASVKHPALMVFQCCNPVYRNSRAQNAGPNCDFKISAPDLLGALQLTRKLWTDSFPDTPLPKLLIPEFKWLAKYQFRNVSLPAGHAETRQNPFDF

SEQ ID NO: 20 - protein, 802 aa, synthetic construct
  REGION 1..802: note = SOURCE - /mol_type="protein" /organism="Chimpanzee adenovirus AdY25"
METQPSPTSPSAPTAGDEKQQQQNESLTAPPPSPASDAAAVPDMQEMEESIEIDLGYVTPAEHEEELAVRFQSSSQEDKEQPEQEAENEQSQAGLEHGDYLHLSGEEDALIKHLARQATIVKDALLDRTEVPLSVEELSRAYELNLFSPRVPPKRQPNGTCEPNPRLNFYPVFAVPEALATYHIFFKNQKIPVSCRANRTRADALFNLGPGARLPDIASLEEVPKIFEGLGSDETRAANALQGEGGGEHEHHSALVELEGDNARLAVLKRTVELTHFAYPALNLPPKVMSAVMDQVLIKRASPISEDEGMQDSEEGKPVVSDEQLARWLGPNATPQSLEERRKLMMAVVLVTVELECLRRFFADAETLRKVEENLHYLFRHGFVRQACKISNVELTNLVSYMGILHENRLGQNVLHTTLRGEARRDYIRDCVYLYLCHTWQTGMGVWQQCLEEQNLKELCKLLQKNLKGLWTGFDERTTASDLADLIFPERLRLTLRNGLPDFMSQSMLQNFRSFILERSGILPATCSALPSDFVPLTFRECPPPLWSHCYLLRLANYLAYHSDVIEDVSGEGLLECHCRCNLCTPHRSLACNPQLLSETQIIGTFELQGPSEGEGAKGGLKLTPGLWTSAYLRKFVPEDYHPFEIRFYEDQSQPPKAELSACVITQGAILAQLQAIQKSRQEFLLKKGRGVYLDPQTGEELNPGFPQDAPRKQEAESGAAARGGFGGRLGEQQSGRGDGGRLGQHSGRGGQPARQSGGRRGGGRGGGGRSSRRQTVVLGGGESKQHGYHLRSGSGSRSAPQ

SEQ ID NO: 21 - protein, 184 aa, synthetic construct
  REGION 1..184: note = SOURCE - /mol_type="protein" /organism="Chimpanzee adenovirus AdY25"
MPRGNKKLKVELPPVEDLEEDWENSSQAEEMEEDWDSTQAEEDSLQDSLEEDEEEAEEEVEEAAAARPSSSAGEKASSTDTISAPGRGPARPHSRWDETGRFPNPTTQTGKKERQGYKSWRGHKNAIVSCLQACGGNISFTRRYLLFHRGVNFPRNILHYYRHLHSPYYFQEEAAAAEKDQKTS

SEQ ID NO: 22 - protein, 219 aa, synthetic construct
  REGION 1..219: note = SOURCE - /mol_type="protein" /organism="Chimpanzee adenovirus AdY25"
MPRGNKKLKVELPPVEDLEEDWENSSQAEEMEEDWDSTQAEEDSLQDSLEEDEEEAEEEVEEAAAARPSSSAGEKASSTDTISAPGRGPARPHSRWDETGRFPNPTTQTAPTTSKKRQQQQKKTRKPARKSTAAAAAGGLRIAANEPAQTRELRNRIFPTLYAIFQQSRGQEQELKVKNRSLRSLTRSCLYHKSEDQLQRTLEDAEALFNKYCALTLKE

SEQ ID NO: 23 - protein, 227 aa, synthetic construct
  REGION 1..227: note = SOURCE - /mol_type="protein" /organism="Chimpanzee adenovirus AdY25"
MSKEIPTPYMWSYQPQMGLAAGAAQDYSTRMNWLSAGPAMISRVNDIRAHRNQILLEQSALTATPRNHLNPRNWPAALVYQEIPQPTTVLLPRDAQAEVQLTNSGVQLAGGATLCRHRPAQGIKRLVIRGRGTQLNDEVVSSSLGLRPDGVFQLAGSGRSSFTPRQAVLTLESSSSQPRSGGIGTLQFVEEFTPSVYFNPFSGSPGHYPDEFIPNFDAISESVDGYD

SEQ ID NO: 24 - protein, 106 aa, synthetic construct
  REGION 1..106: note = SOURCE - /mol_type="protein" /organism="Chimpanzee adenovirus AdY25"
MSHGGAADLARLRHLDHCRRFRCFARDLAEFTYFELPEEHPQGPAHGVRIVVEGGLDSHLLRIFSQRPILVERQQGNTLLTLYCICDHPGLHESLCCLLCTEYNKS

SEQ ID NO: 25 - protein, 212 aa, synthetic construct
  REGION 1..212: note = SOURCE - /mol_type="protein" /organism="Chimpanzee adenovirus AdY25"
MKVFVVCCVLSIIKAEISDYSGLNCGVSASINRSLTFTGNETELQVQCKPHKKYLTWLYQGSPIAVVNHCDDDGVLLNGPANLTFSTRRSKLLLFRPFLPGTYQCISGPCHHTFHLIPNTTSSPAPLPTNNQTNHHQRYRRDLVSESNTTHTGGELRGRKPSGIYYGPWEVVGLIALGLVAGGLLALCYLYLPCFSYLVVLCCWFKKWGRSP

SEQ ID NO: 26 - protein, 176 aa, synthetic construct
  REGION 1..176: note = SOURCE - /mol_type="protein" /organism="Chimpanzee adenovirus AdY25"
MGKITLVCGVLVTVVLSILGGGSAAVVTEKKADPCLTFNPDKCRLSFQPDGNRCAVLIKCGWECESVLVQYKNKTWNNTLASTWQPGDPEWYTVSVPGADGSLRTVNNTFIFEHMCETAMFMSKQYGMWPPRKENIVVFSIAYSACTVLITAIVCLSIHMLIAIRPRNNAEKEKQP

SEQ ID NO: 27 - protein, 209 aa, synthetic construct
  REGION 1..209: note = SOURCE - /mol_type="protein" /organism="Chimpanzee adenovirus AdY25"
MKILSLFCFSIIITSAICNSVDKDVTVTTGSNYTLKGPPSGMLSWYCYFGTDVSQTELCNFQKGKTQNPKIHNYQCNGTDLVLFNITKTYAGSYYCPGDNVDNMIFYELQVVDPTTPAPPTTTTKAHSTDTQETTPEAEVAELAKQIHEDSFVANTPTHPGPQCPGPLVSGIVGVLCGLAVIIICMFIFACCYRRLHRQKSDPLLNLYV

SEQ ID NO: 28 - protein, 302 aa, synthetic construct
  REGION 1..302: note = SOURCE - /mol_type="protein" /organism="Chimpanzee adenovirus AdY25"
MKALSTLVFLTLIGIVFNSKITRVSFLKHVNVTEGNNITLVGVEGAQNTTWTKYHLGWKDICTWNVTYFCIGVNLTIVNANQSQNGLIKGQSVSVTSDGYYTQHNFNYNITVIPLPTPSPPSTTQTTQTTHTTQSSTTTMQTTQTTTYTTSPQPTTTTAEASSSPTIKVAFLMLAPSSSPTASTNEQTTEFLSTIQSSTTATSSAFSSTANLTSLSSMPISNATTSPAPLPTPLKQSESSTQLQITLLIVIGVVILAVLLYFIFCRRIPNAKPAYKPIVIGTPEPLQVEGGLRNLLFSFTVW

SEQ ID NO: 29 - protein, 91 aa, synthetic construct
  REGION 1..91: note = SOURCE - /mol_type="protein" /organism="Chimpanzee adenovirus AdY25"
MIPRHFIITSLICVLQVCATLALVANASPDCIGAFASYVLFAFITCICCCSIVCLLITFFQFVDWVFVRIAYLRHHPQYRDQRVAQLLRLI

SEQ ID NO: 30 - protein, 147 aa, synthetic construct
  REGION 1..147: note = SOURCE - /mol_type="protein" /organism="Chimpanzee adenovirus AdY25"
MRALLLLLALLLAPLAAPLSLKSPTQSPEEVRKCKFQEPWKFLSCYKLKSEMHPSWIMIVGIVNILACTLFSFVIYPRFDFGWNAPEALWLPPDPDTPPQQQQQNQAQAHAPPQPRPQYMPILNYEAEAQRAMLPAISYFNLTGGDD

SEQ ID NO: 31 - protein, 134 aa, synthetic construct
  REGION 1..134: note = SOURCE - /mol_type="protein" /organism="Chimpanzee adenovirus AdY25"
MTDPMANNTVNDLLDMDGRASEQRLAQLRIRQQQERAVKELQDAVAIHQCKRGIFCLVKQAKISFEVTSTDHRLSYELLQQRQKFTCLVGVNPIVITQQSGDTKGCIHCSCDSPECVHTLIKTLCGLRDLLPMN

SEQ ID NO: 32 - protein, 141 aa, synthetic construct
  REGION 1..141: note = SOURCE - /mol_type="protein" /organism="Chimpanzee adenovirus AdY25"
MSGNSSIMTRSRTRLASSRHHPYQPPAPLPRCEETETRASLVEDHPVLPDCDTLSMHNITVIPTTEDSPQLLNFEVQMQECPEGFISLTDPRLARSETVWNVETKTMSITNGIQMFKAVRGERVVYSMSWEGGGKITTRIL

SEQ ID NO: 33 - protein, 299 aa, synthetic construct
  REGION 1..299: note = SOURCE - /mol_type="protein" /organism="Chimpanzee adenovirus AdY25"
MSGNSSIMTRSRTRLASSRHHPYQPPAPLPRCEETETRASLVEDHPVLPDCDTLSMHNVSCVRGLPCSAGFTVLQEFPVPWDMVLTPEELRVLKTCMSVCLCCANIDLFSSQLIHGRERWVLHCHCQDPGSLRCMAGGAVLAIWFRRIIQGCMFNQRVMWYREVVNLHMPKEIMYMGSVFWRGRHLIYIRIWYDGHVGSIVPQMSFGWSTLNYGLLNNLVVLCCTYCSDLSEIRIRCCARRTRRLMLRAIGIMRRESLDPDPLSSSLTERRRQRLLRGLMRHNRPIPFADYDSHRSSSR

SEQ ID NO: 34 - protein, 122 aa, synthetic construct
  REGION 1..122: note = SOURCE - /mol_type="protein" /organism="Chimpanzee adenovirus AdY25"
MVLPVLPSPSVTETQQNCIIWLGLAHSTVADVIRAIRADGIFITQEAQEILHALREWLFYNFNTERSKRRDRRRRAVCSARTRFCFVKYENVRKQLHHDTIQNTISVIPPSSVPTAGPLTSL

SEQ ID NO: 35 - protein, 117 aa, synthetic construct
  REGION 1..117: note = SOURCE - /mol_type="protein" /organism="Chimpanzee adenovirus AdY25"
MRVCLRMPVEGALRELFIMAGLDLPQELIRIIQGWKNENYLGMVQECNMMIEELENAPAFAVLLFLDVRVEALLEATVEHLENRVTFDLAVIFHQHSGGERCHLRDLHFEVLRDRLE

SEQ ID NO: 36 - protein, 129 aa, synthetic construct
  REGION 1..129: note = SOURCE - /mol_type="protein" /organism="Chimpanzee adenovirus AdY25"
MLERTPCTYSIVVPEALNLHLDDFSFVDFLKNCLPDFLSSYLEDITGSSQHAYFNLTFGNAHWGGLRFICNVACPALIPGGPMAKNFGDDMKDYIQLLLREELRDRGRDFDIPIVNLLQVNQEQNLLEL

SEQ ID NO: 37 - protein, 124 aa, synthetic construct
  REGION 1..124: note = SOURCE - /mol_type="protein" /organism="Chimpanzee adenovirus AdY25"
MDAEALYVFLEGAGALLPVQEGSNYIFYAPANFVLHPHGVALLELRLSIVVPRGFIGRFFSLTDANVPGVYASSRIIHAGHREGLSVMLFNHGDSFYEGRAGDPVACLVLERVIYPPVRQASMV

SEQ ID NO: 38 - DNA, 30964 bp, synthetic construct
  misc_feature 1..30964: note = source = /mol_type="DNA" /note="Viral vector based on Chimpanzee adenovirus AdY25" /organism="Artificial Sequence"
  source 1..30964: mol_type = other DNA
ttaatcgcgtttaaacccatcatcaataatatacctcaaactttttgtgcgcgttaatatgcaaatgaggcgtttgaatttgggaagggaggaaggtgattggccgagagaagggcgaccgttaggggcggggcgagtgacgttttgatgacgtgaccgcgaggaggagccagtttgcaagttctcgtgggaaaagtgacgtcaaacgaggtgtggtttgaacacggaaatactcaattttcccgcgctctctgacaggaaatgaggtgtttctaggcggatgcaagtgaaaacgggccattttcgcgcgaaaactgaatgaggaagtgaaaatctgagtaatttcgcgtttatgacagggaggagtatttgccgagggccgagtagactttgaccgattacgtgggggtttcgattaccgtgtttttcacctaaatttccgcgtacggtgtcaaagtccggtgtttttacgtaggtgtcagctgatcgccagggtatttaaacctgcgctctccagtcaagaggccactcttgagtgccagcgagaagagttttctcctccgcgcgcgagtcagatctacactttgaaaggcgatcgctagcgacatcgatcacaagtttgtacaaaaaagctgaacgagaaacgtaaaatgatataaatatcaatatattaaattagattttgcataaaaaacagactacataatactgtaaaacacaacatatccagtcactatggcggccgccgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgtgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcacgcgtggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgatcgattcgacagatcgcgatcgcagtgagtagtgttctggggcgggggaggacctgcatgagggccagaatgactgaaatctgtgcttttctgtgtgttgcagcatcatgagcggaagcggctcctttgagggaggggtattcagcccttatctgacggggcgtctcccctcctgggcgggagtgcgtcagaatgtgatgggatccacggtggacggccggcccgtgcagcccgcgaactcttcaaccctgacctatgcaaccctgagctcttcgtcggtggacgcagctgccgccgcagctgctgcatccgccgccagcgccgtgcgcggaatggccatgggcgccggctactacggcactctggtggccaactcgagttccaccaataatcccgccagcctgaacgaggagaagctgctgctgctgatggcccagcttgaggccttgacccagcgcctgggcgagctgacccagcaggtggctcagctgcaggagcagacgcgggccgcggttgccacggtgaaatccaaataaaaaatgaatcaataaataaacggagacggttgttgattttaacacagagtctgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcgtggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcccttgtgtccgcccaggttttccatgcactcatccatgatgatggcaatgggcccgtgggcggcggcctgggcaaagacgtttcgggggtcggacacatcatagttgtggtcctgggtgaggtcatcataggccattttaatgaatttggggcggagggtgccggactgggggacaaaggtaccctcgatcccgggggcgtagttcccctcacagatctgcatctcccaggctttgagctcagagggggggatcatgtccacctgcggggcgataaagaacacggtttccggggcgggggagatgagctgggccgaaagcaagttccggagcagctgggacttgccgcagccggtggggccgtaaatgaccccgatgaccggctgcaggtggtagttgagggagagacagctgccgtcctcccggaggaggggggccacctcgttcatcatctcgcgcacgtgcatgttctcgcgcaccagttccgccaggaggcgctctccccccagagataggagctcctggagcgaggcgaagtttttcagcggcttgagtccgtcggccatgggcattttggagagggtctgttgcaagagttccaagcggtcccagagctcggtgatgtgctctacggcatctcgatccagcagacctcctcgtttcgcgggttgggacgactgcgggagtagggcaccagacgatgggcgtccagcgcagccagggtccggtccttccagggccgcagcgtccgcgtcagggtggtctccgtcacggtgaaggggtgcgcgccgggctgggcgcttgcgagggtgcgcttcaggctcatccggctggtcgaaaaccgctcccgatcggcgccctgcgcgtcggccaggtagcaattgaccatgagttcgtagttgagcgcctcggccgcgtggcctttggcgcggagcttacctttggaagtctgcccgcaggcgggacagaggagggacttgagggcgtagagcttgggggcgaggaagacggaatcgggggcgtaggcgtccgcgccgcagtgggcgcagacggtctcgcactccacgagccaggtgaggtcgggctggtcggggtcaaaaaccagtttcccgccgttctttttgatgcgtttcttacctttggtctccatgagctcgtgtccccgctgggtgacaaagaggctgtccgtgtccccgtagaccgactttatgggccggtcctcgagcggtgtgccgcggtcctcctcgtagaggaaccccgcccactccgagacgaaagcccgggtccaggccagcacgaaggaggccacgtgggacgggtagcggtcgttgtccaccagcgggtccactttttccagggtatgcaaacacatgtccccctcgtccacatccaggaaggtgattggcttgtaagtgtaggccacgtgaccgggggtcccggccgggggggtataaaagggggcgggcccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccagcggagatgcctttcaagagcccctcgtccatctggtcagaaaagacgatttttttgttgtcgagcttggtggcgaaggagccgtagagggcgttggaaaggagcttggcgatggagcgcatggtctggtttttttccttgtcggcgcgctccttggccgcgatgttgagctgcacgtactcgcgcgccacgcacttccattcggggaagacggtggtcatctcgtcgggcacgattctgacctgccaacctcgattatgcagggtgatgaggtccacactggtggccacctcgccgcgcaggggctcgttggtccagcagaggcggccgcccttgcgcgagcagaaggggggcagagggtccagcatgacctcgtcgggggggtcggcatcgatggtgaagatgccgggcaggagatcggggtcgaagtagctgatggaagtggccagatcgtccagggaagcttgccattcgcgcacggccagcgcgcgctcgtagggactgaggggcgtgccccagggcatggggtgggtgagcgcggaggcgtacatgccgcagatgtcgtagacgtagaggggctcctcgaggatgccgatgtaggtggggtagcagcgccccccgcggatgctggcgcgcacgtagtcatacagctcgtgcgagggcgcgaggagccccgggcccaggttggtgcgactgggcttttcggcgcggtagacgatctggcgaaagatggcatgcgagttggaggagatggtgggcctttggaagatgttgaagtgggcgtgggggaggccgaccgagtcgcggatgaagtgggcgtaggagtcttgcagtttggcgacgagctcggcggtgacgaggacgtccagagcgcagtagtcgagggtctcctggatgatgtcatacttgagctggcccttttgtttccacagctcgcggttgagaaggaactcttcgcggtccttccagtactcttcgagggggaacccgtcctgatctgcacggtaagagcctagcatgtagaactggttgacggccttgtaggcgcagcagcccttctccacggggagggcgtaggcctgggcggccttgcgcagggaggtgtgcgtgagggcgaaggtgtccctgaccatgaccttgaggaactggtgcttgaaatcgatatcgtcgcagcccccctgctcccagagctggaagtccgtgcgcttcttgtaggcggggttgggcaaagcgaaagtaacatcgttgaaaaggatcttgcccgcgcggggcataaagttgcgagtgatgcggaaaggctggggcacctcggcccggttgttgatgacctgggcggcgagcacgatctcgtcgaaaccgttgatgttgtggcccacgatgtagagttccacgaatcgcgggcggcccttgacgtggggcagcttcttgagctcctcgtaggtgagctcgtcggggtcgctgagaccgtgctgctcgagcgcccagtcggcgagatgggggttggcgcggaggaaggaagtccagagatccacggccagggcggtttgcagacggtcccggtactgacggaactgctgcccgacggccattttttcgggggtgacgcagtagaaggtgcgggggtccccgtgccagcggtcccatttgagctggagggcgagatcgagggcgagctcgacgaggcggtcgtcccctgagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccgttggtggcgacgtcgatggcttgcagggtcccgtgcccctggggtgtgaccaccgtcccccgtttcttcttgggcggctggggcgacgggggcggtgcctcttccatggttagaagcggcggcgaggacgcgcgccgggcggcagaggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcctgaaggtctccgcgaccggcgcgctccacggtggccgcgaggtcgttggagatgcggcccatgagctgcgagaaggcgttcatgcccgcctcgttccagacgcggctgtagaccacgacgccctcgggatcgcgggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaagcgttccatggcctcgtaaaagtccacggcgaagttgaaaaactgggagttgcgcgccgagacggtcaactcctcctccagaagacggatgagctcggcgatggtggcgcgcacctcgcgctcgaaggcccccgggagttcctccacttcctcctcttcttcctcctccactaacatctcttctacttcctcctcaggcggtggtggtggcgggggagggggcctgcgtcgccggcggcgcacgggcagacggtcgatgaagcgctcgatggtctcgccgcgccggcgtcgcatggtctcggtgacggcgcgcccgtcctcgcggggccgcagcgtgaagacgccgccgcgcatctccaggtggccgggggggtccccgttgggcagggagagggcgctgacgatgcatcttatcaattgccccgtagggactccgcgcaaggacctgagcgtctcgagatccacgggatctgaaaaccgttgaacgaaggcttcgagccagtcgcagtcgcaaggtaggctgagcacggtttcttctgccgggtcatgttggggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccaggtccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcctgctggatctgggtgagggtggtctggaagtcgtcaaagtcgacgaagcggtggtaggctccggtgttgatggtgtaggagcagttggccatgacggaccagttgacggtctggtggcccggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtactggcactcccgtctcgacccaagcctgcaccaaccctccaggatacggaggcgggtcgttttgcaactttttttggaggccggaaatgaaactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagaccccatagccagccgacttctccagttacggagcgagcccctcttttgttttgtttgtttttgccagatgcatcccgtactgcggcagatgcgcccccaccaccctccaccgcaacaacagccccctcctccacagccggcgcttctgcccccgccccagcagcagcagcaacttccagccacgaccgccgcggccgccgtgagcggggctggacagacttctcagtatgatcacctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaggcgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagtctgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgcgcggtgagcgccagcaggcggcgcgagctgagcgaccaggagctgatgcacagcctgcagcgggccctgaccggggccgggaccgagggggagagctactttgacatgggcgcggacctgcactggcagcccagccgccgggccttggaggcggcaggcggtcccccctacatagaagaggtggacgatgaggtggacgaggagggcgagtacctggaagactgatggcgcgaccgtatttttgctagatgcaacaacagccacctcctgatcccgcgatgcgggcggcgctgcagagccagccgtccggcattaactcctcggacgattggacccaggccatgcaacgcatcatggcgctgacgacccgcaaccccgaagcctttagacagcagccccaggccaaccggctctcggccatcctggaggccgtggtgccctcgcgctccaaccccacgcacgagaaggtcctggccatcgtgaacgcgctggtggagaacaaggccatccgcggcgacgaggccggcctggtgtacaacgcgctgctggagcgcgtggcccgctacaacagcaccaacgtgcagaccaacctggaccgcatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggcgttcaagaacttgcagggcctgtggggcgtgcaggccccggtcggggaccgcgcgacggtgtcgagcctgctgacgccgaactcgcgcctgctgctgctgctggtggcccccttcacggacagcggcagcatcaaccgcaactcgtacctgggctacctgattaacctgtaccgcgaggccatcggccaggcgcacgtggacgagcagacctaccaggagatcacccacgtgagccgcgccctgggccaggacgacccgggcaatctggaagccaccctgaactttttgctgaccaaccggtcgcagaagatcccgccccagtacacgctcagcgccgaggaggagcgcatcctgcgatacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctgaatccccactggctcccgccgccggggttctacacgggcgagtacgacatgcccgaccccaatgacgggttcctgtgggacgatgtggacagcagcgtgttctccccccgaccgggtgctaacgagcgccccttgtggaagaaggaaggcagcgaccgacgcccgtcctcggcgctgtccggccgcgagggtgctgccgcggcggtgcccgaggccgccagtcctttcccgagcttgcccttctcgctgaacagtattcgcagcagcgagctgggcaggatcacgcgcccgcgcttgctgggcgaggaggagtacttgaatgactcgctgttgagacccgagcgggagaagaacttccccaataacgggatagagagcctggtggacaagatgagccgctggaagacgtatgcgcaggagcacagggacgatccgtcgcagggggccacgagccggggcagcgccgcccgtaaacgccggtggcacgacaggcagcggggactgatgtgggacgatgaggattccgccgacgacagcagcgtgttggacttgggtgggagtggtaacccgttcgctcacctgcgcccccgcatcgggcgcatgatgtaagagaaaccgaaaataaatgatactcaccaaggccatggcgaccagcgtgcgttcgtttcttctctgttgttgtatctagtatgatgaggcgtgcgtacccggagggtcctcctccctcgtacgagagcgtgatgcagcaggcgatggcggcggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggtcagctgaaaaccatcatgcacaccaacatgcccaacgtgaacgagttcatgtacagcaacaagttcaaggcgcgggtgatggtctcccgcaagacccccaacggggtgacagtgacagatggtagtcaggatatcttggagtatgaatgggtggagtttgagctgcccgaaggcaacttctcggtgaccatgaccatcgacctgatgaacaacgccatcatcgacaattacttggcggtggggcggcagaacggggtcctggagagcgatatcggcgtgaagttcgacactaggaacttcaggctgggctgggaccccgtgaccgagctggtcatgcccggggtgtacaccaacgaggccttccaccccgatattgtcttgctgcccggctgcggggtggacttcaccgagagccgcctcagcaacctgctgggcattcgcaagaggcagcccttccaggagggcttccagatcatgtacgaggatctggaggggggcaacatccccgcgctcctggatgtcgacgcctatgagaaaagcaaggaggagagcgccgccgcggcgactgcagctgtagccaccgcctctaccgaggtcaggggcgataattttgccagccctgcagcagtggcagcggccgaggcggctgaaaccgaaagtaagatagtcattcagccggtggagaaggatagcaaggacaggagctacaacgtgctgccggacaagataaacaccgcctaccgcagctggtacctggcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgctcacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaaaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcggtcgcgcaccaccgtcgacgacgtgatcgaccaggtggtggccgacgcgcgcaactacacccccgccgccgcgcccgtctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcttcaggcgccagcgccggcaggacccggagacgcgcggccacggcggcggcagcggccatcgccagcatgtcccgcccgcggcgagggaacgtgtactgggtgcgcgacgccgccaccggtgtgcgcgtgcccgtgcgcacccgcccccctcgcacttgaagatgttcacttcgcgatgttgatgtgtcccagcggcgaggaggatgtccaagcgcaaattcaaggaagagatgctccaggtcatcgcgcctgagatctacggccccgcggtggtgaaggaggaaagaaagccccgcaaaatcaagcgggtcaaaaaggacaaaaaggaagaagatgacgatctggtggagtttgtgcgcgagttcgccccccggcggcgcgtgcagtggcgcgggcggaaagtgcacccggtgctgagacccggcaccaccgtggtcttcacgcccggcgagcgctccggcagcgcttccaagcgctcctacgacgaggtgtacggggacgaggacatcctcgagcaggcggccgagcgcctgggcgagtttgcttacggcaagcgcagccgccccgccctgaaggaagaggcggtgtccatcccgctggaccacggcaaccccacgccgagcctcaagcccgtgaccctgcagcaggtgctgccgagcgcagcgccgcgccgggggttcaagcgcgagggcgaggatctgtaccccaccatgcagctgatggtgcccaagcgccagaagctggaagacgtgctggagaccatgaaggtggacccggacgtgcagcccgaggtcaaggtgcggcccatcaagcaggtggccccgggcctgggcgtgcagaccgtggacatcaagatccccacggagcccatggaaacgcagaccgagcccatgatcaagcccagcaccagcaccatggaggtgcagacggatccctggatgccatcggctcctagccgaagaccccggcgcaagtacggcgcggccagcctgctgatgcccaactacgcgctgcatccttccatcatccccacgccgggctaccgcggcacgcgcttctaccgcggtcatacaaccagccgccgccgcaagaccaccacccgccgccgccgtcgccgcacagccgctgcatctacccctgccgccctggtgcggagagtgtaccgccgcggccgcgcgcctctgaccctaccgcgcgcgcgctaccacccgagcatcgccatttaaactttcgcctgctttgcagatggccctcacatgccgcctccgcgttcccattacgggctaccgaggaagaaaaccgcgccgtagaaggctggcggggaacgggatgcgtcgccaccaccatcggcggcggcgcgccatcagcaagcggttggggggaggcttcctgcccgcgctgatccccatcatcgccgcggcgatcggggcgatccccggcattgcttccgtggcggtgcaggcctctcagcgccactgagacacttggaaaacatcttgtaataaaccaatggactctgacgctcctggtcctgtgatgtgttttcgtagacagatggaagacatcaatttttcgtccctggctccgcgacacggcacgcggccgttcatgggcacctggagcgacatcggcaccagccaactgaacgggggcgccttcaattggagcagtctctggagcgggcttaagaatttcgggtccacgcttaaaacctatggcagcaaggcgtggaacagcaccacagggcaggcgctgagggataagctgaaagagcagaacttccagcagaaggtggtcgatgggctcgcctcgggcatcaacggggtggtggacctggccaaccaggccgtgcagcggcagatcaacagccgcctggacccggtgccgcccgccggctccgtggagatgccgcaggtggaggaggagctgcctcccctggacaagcggggcgagaagcgaccccgccccgacgcggaggagacgctgctgacgcacacggacgagccgcccccgtacgaggaggcggtgaaactgggtctgcccaccacgcggcccatcgcgcccctggccaccggggtgctgaaacccgaaagtaataagcccgcgaccctggacttgcctcctcccgcttcccgcccctctacagtggctaagcccctgccgccggtggccgtggcccgcgcgcgacccgggggctccgcccgccctcatgcgaactggcagagcactctgaacagcatcgtgggtctgggagtgcagagtgtgaagcgccgccgctgctattaaacctaccgtagcgcttaacttgcttgtctgtgtgtgtatgtattatgtcgccgctgtccgccagaaggaggagtgaagaggcgcgtcgccgagttgcaagatggccaccccatcgatgctgccccagtgggcgtacatgcacatcgccggacaggacgcttcggagtacctgagtccgggtctggtgcagttcgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccctagcttcaaaccctactccggcaccgcctacaacagcctggctcccaagggagcgcccaattccagccagtgggagcaaaaaaaggcaggcaatggtgacactatggaaacacacacatttggtgtggccccaatgggcggtgagaatattacaatcgacggattacaaattggaactgacgctacagctgatcaggataaaccaatttatgctgacaaaacattccagcctgaacctcaagtaggagaagaaaattggcaagaaactgaaagcttttatggcggtagggctcttaaaaaagacacaagcatgaaaccttgctatggctcctatgctagacccaccaatgtaaagggaggtcaagctaaacttaaagttggagctgatggagttcctaccaaagaatttgacatagacctggctttctttgatactcccggtggcacagtgaatggacaagatgagtataaagcagacattgtcatgtataccgaaaacacgtatctggaaactccagacacgcatgtggtatacaaaccaggcaaggatgatgcaagttctgaaattaacctggttcagcagtccatgcccaatagacccaactatattgggttcagagacaactttattgggctcatgtattacaacagtactggcaatatgggggtgctggctggtcaggcctcacagctgaatgctgtggtcgacttgcaagacagaaacaccgagctgtcataccagctcttgcttgactctttgggtgacagaacccggtatttcagtatgtggaatcaggcggtggacagttatgatcctgatgtgcgcattattgaaaaccatggtgtggaagacgaacttcccaactattgcttccccctggatgggtctggcactaatgccgcttaccaaggtgtgaaagtaaaaaatggtaacgatggtgatgttgagagcgaatgggaaaatgatgatactgtcgcagctcgaaatcaattatgcaagggcaacatttttgccatggaaattaacctccaagccaacctgtggagaagtttcctctactcgaacgtggccctgtacctgcccgactcttacaagtacacgccagccaacatcaccctgcccaccaacaccaacacttatgattacatgaacgggagagtggtgcctccctcgctggtggacgcctacatcaacatcggggcgcgctggtcgctggaccccatggacaacgtcaatcccttcaaccaccaccgcaacgcgggcctgcgctaccgctccatgctcctgggcaacgggcgctacgtgcccttccacatccaggtgccccagaaatttttcgccatcaagagcctcctgctcctgcccgggtcctacacctacgagtggaacttccgcaaggacgtcaacatgatcctgcagagctccctcggcaacgacctgcgcacggacggggcctccatctccttcaccagcatcaacctctacgccaccttcttccccatggcgcacaacacggcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgcctcaagaccaaggagacgccctcgctgggctccgggttcgacccctacttcgtctactcgggctccatcccctacctcgacggcaccttctacctcaaccacaccttcaagaaggtctccatcaccttcgactcctccgtcagctggcccggcaacgaccggctcctgacgcccaacgagttcgaaatcaagcgcaccgtcgacggcgagggatacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtggtggacgaggtcaactacaaggactaccaggccgtcaccctggcctaccagcacaacaactcgggcttcgtcggctacctcgcgcccaccatgcgccagggccagccctaccccgccaactacccgtacccgctcatcggcaagagcgccgtcaccagcgtcacccagaaaaagttcctctgcgacagggtcatgtggcgcatccccttctccagcaacttcatgtccatgggcgcgctcaccgacctcggccagaacatgctctatgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacccccttctcggccggtaacgccaccacctaaattgctacttgcatgatggctgagcccacaggctccggcgagcaggagctcagggccatcatccgcgacctgggctgcgggccctacttcctgggcaccttcgataagcgcttcccgggattcatggccccgcacaagctggcctgcgccatcgtcaacacggccggccgcgagaccgggggcgagcactggctggccttcgcctggaacccgcgctcgaacacctgctacctcttcgaccccttcgggttctcggacgagcgcctcaagcagatctaccagttcgagtacgagggcctgctgcgccgtagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaactcccactccgcctactttcgctcccaccgcgcgcgcatcgagaaggccaccgccttcgaccgcatgaacaatcaagacatgtaaaccgtgtgtgtatgtttaaaatatcttttaataaacagcactttaatgttacacatgcatctgagatgattttattttagaaatcgaaagggttctgccgggtctcggcatggcccgcgggcagggacacgttgcggaactggtacttggccagccacttgaactcggggatcagcagtttgggcagcggggtgtcggggaaggagtcggtccacagcttccgcgtcagctgcagggcgcccagcaggtcgggcgcggagatcttgaaatcgcagttgggacccgcgttctgcgcgcgagagttgcggtacacggggttgcagcactggaacaccatcagggccgggtgcttcacgctcgccagcaccgccgcgtcggtgatgctctccacgtcgaggtcctcggcgttggccatcccgaagggggtcatcttgcaggtctgccttcccatggtgggcacgcacccgggcttgtggttgcaatcgcagtgcagggggatcagcatcatctgggcctggtcggcgttcatccccgggtacatggccttcatgaaagcctccaattgcctgaacgcctgctgggccttggctccctcggtgaagaagaccccgcaggacttgctagagaactggttggtggcacagccggcatcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcatgtgctccttctggatcatggtggtcccgtgcaggcaccgcagtttgccctcggcctcggtgcacccgtgcagccacagcgcgcacccggtgcactcccagttcttgtgggcgatctgggaatgcgcgtgcacgaacccttgcaggaagcggcccatcatggtcgtcagggtcttgttgctagtgaaggtcaacgggatgccgcggtgctcctcgttgatgtacaggtggcagatgcggcggtacacctcgccctgctcgggcatcagttggaagttggctttcaggtcggtctccacgcggtagcggtccatcagcatagtcatgatttccatgcccttctcccaggccgagacgatgggcaggctcatagggttcttcaccatcatcttagcactagcagccgcggccagggggtcgctctcatccagggtctcaaagctccgcttgccgtccttctcggtgatccgcaccggggggtagctgaagcccacggccgccagctcctcctcggcctgtctttcgtcctcgctgtcctggctgacgtcctgcatgaccacatgcttggtcttgcggggtttcttcttgggcggcagtggcggcggagatgcttgtggcgagggggagcgcgagttctcgctcaccactactatctcttcctcttcttggtccgaggccacgcggcggtaggtatgtctcttcgggggcagaggcggaggcgacgggctctcgccgccgcgacttggcggatggctggcagagccccttccgcgttcgggggtgcgctcccggcggcgctctgactgacttcctccgcggccggccattgtgttctcctagggaggaacaacaagcatggagactcagccatcgccaacctcgccatctgcccccaccgccggcgacgagaagcagcagcagcagaatgaaagcttaaccgccccgccgcccagccccgcctccgacgcagccgcggtcccagacatgcaagagatggaggaatccatcgagattgacctgggctatgtgacgcccgcggagcatgaggaggagctggcagtgcgctttcaatcgtcaagccaggaagataaagaacagccagagcaggaagcagagaacgagcagagtcaggctgggctcgagcatggcgactacctccacctgagcggggaggaggacgcgctcatcaagcatctggcccggcaggccaccatcgtcaaggacgcgctgctcgaccgcaccgaggtgcccctcagcgtggaggagctcagccgcgcctacgagctcaacctcttctcgccgcgcgtgccccccaagcgccagcccaacggcacctgcgagcccaacccccgcctcaacttctacccggtcttcgcggtgcccgaggccctggccacctaccacatctttttcaagaaccaaaagatccccgtctcctgccgcgccaaccgcacccgcgccgacgccctcttcaacctgggtcccggcgcccgcctacctgatatcgcctccttggaagaggttcccaagatcttcgagggtctgggcagcgacgagactcgggccgcgaacgctctgcaaggagaaggaggaggagagcatgagcaccacagcgccctggtcgagttggaaggcgacaacgcgcggctggcggtgctcaaacgcacggtcgagctgacccatttcgcctacccggctctgaacctgcccccgaaagtcatgagcgcggtcatggaccaggtgctcatcaagcgcgcgtcgcccatctccgaggacgagggcatgcaagactccgaggagggcaagcccgtggtcagcgacgagcagctggcccggtggctgggtcctaatgctacccctcaaagtttggaagagcggcgcaagctcatgatggccgtggtcctggtgaccgtggagctggagtgcctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcttgcacgagaaccgcctggggcagaacgtgctgcacaccaccctgcgcggggaggcccgccgcgactacatccgcgactgcgtctacctctacctctgccacacctggcagacgggcatgggcgtgtggcagcagtgtctggaggagcagaacctgaaagagctctgcaagctcctgcaaaagaacctcaagggtctgtggaccgggttcgacgagcggaccaccgcctcggacctggccgacctcatcttccccgagcgcctcaggctgacgctgcgcaacggcctgcccgactttatgagccaaagcatgttgcaaaactttcgctctttcatcctcgaacgctccggaatcctgcccgccacctgctccgcgctgccctcggacttcgtgccgctgaccttccgcgagtgccccccgccgctgtggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaagggcccagcgagggcgagggagccaaggggggtctgaaactcaccccggggctgtggacctcggcctacttgcgcaagttcgtgcccgaggattaccatcccttcgagatcaggttctacgaggaccaatcccagccgcccaaggccgagctgtcggcctgcgtcatcacccagggggcgatcctggcccaattgcaagccatccagaaatcccgccaagaattcttgctgaaaaagggccgcggggtctacctcgacccccagaccggtgaggagctcaaccccggcttcccccaggatgccccgaggaaacaagaagctgaaagtggagctgccgcccgtggaggatttggaggaagactgggagaacagcagtcaggcagaggagatggaggaagactgggacagcactcaggcagaggaggacagcctgcaagacagtctggaggaagacgaggaggaggcagaggaggaggtggaagaagcagccgccgccagaccgtcgtcctcggcgggggagaaagcaagcagcacggataccatctccgctccgggtcggggtcccgctcggccccacagtagatgggacgagaccgggcgattcccgaaccccaccacccagaccggtaagaaggagcggcagggatacaagtcctggcgggggcacaaaaacgccatcgtctcctgcttgcaggcctgcgggggcaacatctccttcacccggcgctacctgctcttccaccgcggggtgaacttcccccgcaacatcttgcattactaccgtcacctccacagcccctactacttccaagaagaggcagcagcagcagaaaaagaccagaaaaccagctagaaaatccacagcggcggcagcggcaggtggactgaggatcgcggcgaacgagccggcgcagacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccagtcgcagaaaaaggcgggaattacgtcacctgtgcccttcgccctagccgcctccacccagcaccgccatgagcaaagagattcccacgccttacatgtggagctaccagccccagatgggcctggccgccggcgccgcccaggactactccacccgcatgaattggctcagcgccgggcccgcgatgatctcacgggtgaatgacatccgcgcccaccgaaaccagatactcctagaacagtcagcgctcaccgccacgccccgcaatcacctcaatccgcgtaattggcccgccgccctggtgtaccaggaaattccccagcccacgaccgtactacttccgcgagacgcccaggccgaagtccagctgactaactcaggtgtccagctggcgggcggcgccaccctgtgtcgtcaccgccccgctcagggtataaagcggctggtgatccggggcagaggcacacagctcaacgacgaggtggtgagctcttcgctgggtctgcgacctgacggagtcttccaactcgccggatcggggagatcttccttcacgcctcgtcaggcggtcctgactttggagagttcgtcctcgcagccccgctcgggcggcatcggcactctccagttcgtggaggagttcactccctcggtctacttcaaccccttctccggctcccccggccactacccggacgagttcatcccgaactttgacgccatcagcgagtcggtggacggctacgattgattaattaatcaactaaccccttacccctttaccctccagtaaaaataaagattaaaaatgattgaattgatcaataaagaatcacttacttgaaatctgaaaccaggtctctgtccatgttttctgtcagcagcacttcactcccctcttcccaactctggtactgcaggccccggcgggctgcaaacttcctccacactctgaaggggatgtcaaattcctcctgtccctcaatcttcatttttatcttctatcagatgtccaaaaagcgcgcgcgggtggatgatggcttcgaccccgtgtacccctacgatgcagacaacgcaccgactgtgcccttcatcaaccctcccttcgtctcttcagatggattccaagaaaagcccctgggggtgttgtccctgcgactggccgaccccgtcaccaccaagaatggggctgtcaccctcaagctgggggagggggtggacctcgacgactcgggaaaactcatctccaaaaatgccaccaaggccactgcccctctcagtatttccaacggcaccatttcccttaacatggctgcccctttttacaacaacaatggaacgttaagtctcaatgtttctacaccattagcagtatttcccacttttaacactttaggtatcagtcttggaaacggtcttcaaacttctaataagttgctgactgtacagttaactcatcctcttacattcagctcaaatagcatcacagtaaaaacagacaaaggactctatattaattctagtggaaacagagggcttgaggctaacataagcctaaaaagaggactgatttttgatggtaatgctattgcaacataccttggaagtggtttagactatggatcctatgatagcgatgggaaaacaagacccatcatcaccaaaattggagcaggtttgaattttgatgctaataatgccatggctgtgaagctaggcacaggtttaagttttgactctgccggtgccttaacagctggaaacaaagaggatgacaagctaacactttggactacacctgacccaagccctaattgtcaattactttcagacagagatgccaaatttaccctatgtcttacaaaatgcggtagtcaaatactaggcactgttgcagtagctgctgttactgtaggttcagcactaaatccaattaatgacacagtaaaaagcgccatagtattccttagatttgactctgacggtgtgctcatgtcaaactcatcaatggtaggtgattactggaactttagggaaggacagaccacccaaagtgtggcctatacaaatgctgtgggattcatgcccaatctaggtgcatatcctaaaacccaaagcaaaacaccaaaaaatagtatagtaagtcaggtatatttaaatggagaaactactatgccaatgacactgacaataactttcaatggcactgatgaaaaagacacaacacctgtgagcacttactccatgacttttacatggcagtggactggagactataaggacaagaatattacctttgctaccaactcctttactttctcctacatggcccaagaataaaccctgcatgccaaccccattgttcccaccactatggaaaactctgaagcagaaaaaaataaagttcaagtgttttattgattcaacagttttcacagaattcgagtagttattttccctcctccctcccaactcatggaatacaccaccctctccccacgcacagccttaaacatctgaatgccattggtaatggacatggttttggtctccacattccacacagtttcagagcgagccagtctcgggtcggtcagggagatgaaaccctccgggcactcctgcatctgcacctcaaagttcagtagctgagggctgtcctcggtggtcgggatcacagttatctggaagaagagcggtgagagtcataatccgcgaacgggatcgggcggttgtggcgcatcaggccccgcagcagtcgctgtctgcgccgctccgtcaagctgctgctcaaggggtctgggtccagggactccctgcgcatgatgccgatggccctgagcatcagtcgcctggtgcggcgggcgcagcagcggatgcggatctcactcaggtcggagcagtacgtgcagcacagcactaccaagttgttcaacagtccatagttcaacgtgctccagccaaaactcatctgtggaactatgctgcccacatgtccatcgtaccagatcctgatgtaaatcaggtggcgccccctccagaacacactgcccatgtacatgatctccttgggcatgtgcaggttcaccacctcccggtaccacatcacccgctggttgaacatgcagccctggataatcctgcggaaccagatggccagcaccgccccgcccgccatgcagcgcagggaccccgggtcctggcaatggcagtggagcacccaccgctcacggccgtggattaactgggagctgaacaagtctatgttggcacagcacaggcacacgctcatgcatgtcttcagcactctcagttcctcgggggtcaggaccatgtcccagggcacggggaactcttgcaggacagtgaacccggcagaacagggcagccctcgcacacaacttacattgtgcatggacagggtatcgcaatcaggcagcaccggatgatcctccaccagagaagcgcgggtctcggtctcctcacagcgaggtaagggggccggcggttggtacggatgatggcgggatgacgctaatcgtgttctggatcgtgtcatgatggagctgtttcctgacattttcgtacttcacgaagcagaacctggtacgggcactgcacaccgctcgccggcgacggtctcggcgcttcgagcgctcggtgttgaagttatagaacagccactccctcagagcgtgcagtatctcctgagcctcttgggtgatgaaaatcccatccgctctgatggctctgatcacatcggccacggtggaatgggccagacccagccagatgatgcaattttgttgggtttcggtgacggagggagagggaagaacaggaagaaccatgattaactttattccaaacggtctcggagcacttcaaaatgcaggtcccggaggtggcacctctcgcccccactgtgttggtggaaaataacagccaggtcaaaggtgacacggttctcgagatgttccacggtggcttccagcaaagcctccacgcgcacatccagaaacaagaggacagcgaaagcgggagcgttttctaattcctcaatcatcatattacactcctgcaccatccccagataattttcatttttccagccttgaatgattcgtattagttcctgaggtaaatccaagccagccatgataaaaagctcgcgcagagcgccctccaccggcattcttaagcacaccctcataattccaagagattctgctcctggttcacctgcagcagattaacaatgggaatatcaaaatctctgccgcgatccctaagctcctccctcaacaataactgtatgtaatctttcatatcatctccgaaatttttagccatagggccgccaggaataagagcagggcaagccacattacagataaagcgaagtcctccccagtgagcattgccaaatgtaagattgaaataagcatgctggctagaccctgtgatatcttccagataactggacagaaaatcaggcaagcaatttttaagaaaatcaacaaaagaaaagtcgtccaggtgcaggtttagagcctcaggaacaacgatggaataagtgcaaggagtgcgttccagcatggttagtgtttttttggtgatctgtagaacaaaaaataaacatgcaatattaaaccatgctagcctggcgaacaggtgggtaaatcactctttccagcaccaggcaggctacggggtctccggcgcgaccctcgtagaagctgtcgccatgattgaaaagcatcaccgagagaccttcccggtggccggcatggatgattcgagaagaagcatacactccgggaacattggcatccgtgagtgaaaaaaagcgacctataaagcctcggggcactacaatgctcaatctcaattccagcaaagccaccccatgcggatggagcacaaaattggcaggtgcgtaaaaaatgtaattactcccctcctgcacaggcagcaaagcccccgctccctccagaaacacatacaaagcctcagcgtccatagcttaccgagcacggcaggcgcaagagtcagagaaaaggctgagctctaacctgactgcccgctcctgtgctcaatatatagccctaacctacactgacgtaaaggccaaagtctaaaaatacccgccaaaatgacacacacgcccagcacacgcccagaaaccggtgacacactcaaaaaaatacgtgcgcttcctcaaacgcccaaaccggcgtcatttccgggttcccacgctacgtcaccgctcagcgactttcaaattccgtcgaccgttaaaaacgtcactcgccccgcccctaacggtcgcccttctctcggccaatcaccttcctcccttcccaaattcaaacgcctcatttgcatattaacgcgcacaaaaagtttgaggtatatatttgaatgatg

SEQ ID NO: 39 - protein, 150 aa, synthetic construct
  REGION 1..150: note = SOURCE - /mol_type="protein" /organism="Human adenovirus AdHu5"
MTTSGVPFGMTLRPTRSRLSRRTPYSRDRLPPFETETRATILEDHPLLPECNTLTMHNAWTSPSPPVKQPQVGQQPVAQQLDSDMNLSELPGEFINITDERLARQETVWNITPKNMSVTHDMMLFKASRGERTVYSVCWEGGGRLNTRVL

SEQ ID NO: 40 - protein, 294 aa, synthetic construct
  REGION 1..294: note = SOURCE - /mol_type="protein" /organism="Human adenovirus AdHu5"
MTTSGVPFGMTLRPTRSRLSRRTPYSRDRLPPFETETRATILEDHPLLPECNTLTMHNVSYVRGLPCSVGFTLIQEWVVPWDMVLTREELVILRKCMHVCLCCANIDIMTSMMIHGYESWALHCHCSSPGSLQCIAGGQVLASWFRMVVDGAMFNQRFIWYREVVNYNMPKEVMFMSSVFMRGRHLIYLRLWYDGHVGSVVPAMSFGYSALHCGILNNIVVLCCSYCADLSEIRVRCCARRTRRLMLRAVRIIAEETTAMLYSCRTERRRQQFIRALLQHHRPILMHDYDSTPM

SEQ ID NO: 41 - protein, 114 aa, synthetic construct
  REGION 1..114: note = SOURCE - /mol_type="protein" /organism="Human adenovirus AdHu5"
MVLPALPAPPVCDSQNECVGWLGVAYSAVVDVIRAAAHEGVYIEPEARGRLDALREWIYYNYYTERSKRRDRRRRSVCHARTWFCFRKYDYVRRSIWHDTTTNTISVVSAHSVQ

SEQ ID NO: 42 - DNA, 1107 bp, synthetic construct
  sig_peptide 1..102: note = /note="signal peptide from human tissue plasminogen activator (tPA)"
  misc_feature 1072..1104: note = /note="PK tag"
  misc_feature 1..1107: note = source = /mol_type="DNA" /note="Ag85A" /organism="Mycobacterium tuberculosis"
  source 1..1107: mol_type = other DNA
  CDS 1..1107
atggacgccatgaagaggggcctgtgctgcgtgctgctgctgtgtggcgccgtgttcgtgtcccccagccaggaaatccacgcccggttcagacggggcagcatgcagctggtggacagagtcagaggcgccgtgaccggcatgagcagacggctggtcgtgggagctgtcggagccgctctggtgtctggactcgtgggagccgtgggcggaacagctacagccggcgctttcagcagacccggcctgcccgtggaatatctgcaggtccccagccccagcatgggccgggacatcaaggtgcagttccagtctggcggagccaacagccctgctctgtacctgctggacggcctgagagcccaggacgacttcagcggctgggacatcaacacccccgccttcgagtggtacgaccagagcggcctgtctgtggtcatgcctgtgggcggccagagcagcttctacagcgactggtatcagcccgcttgtggcaaggccggctgccagacctacaagtgggagacattcctgaccagcgagctgcccggctggctgcaggccaacagacacgtgaagcccaccggctctgccgtcgtgggcctgtctatggctgccagctctgccctgaccctggccatctaccacccccagcagttcgtgtacgctggcgccatgtctggcctgctggatccttctcaggccatgggacccaccctgatcggactggctatgggagatgccggcggatacaaggccagcgacatgtggggccctaaagaggaccccgcctggcagagaaacgaccccctgctgaacgtgggcaagctgatcgccaacaacaccagagtgtgggtgtactgcggcaacggcaagctgagcgacctgggcggcaacaacctgcccgccaagttcctggaaggcttcgtgcggaccagcaacatcaagttccaggacgcctacaacgctggcggcggacacaacggcgtgttcgacttccccgacagcggcacccacagctgggagtattggggagcccagctgaatgccatgaagcccgacctgcagagaggcagcatccctaatcctctgctgggcctggactga

SEQ ID NO: 43 - protein, 368 aa, synthetic construct
  REGION 1..368: note = SOURCE - /mol_type="protein" /note="Ag85A" /organism="Mycobacterium tuberculosis"
MDAMKRGLCCVLLLCGAVFVSPSQEIHARFRRGSMQLVDRVRGAVTGMSRRLVVGAVGAALVSGLVGAVGGTATAGAFSRPGLPVEYLQVPSPSMGRDIKVQFQSGGANSPALYLLDGLRAQDDFSGWDINTPAFEWYDQSGLSVVMPVGGQSSFYSDWYQPACGKAGCQTYKWETFLTSELPGWLQANRHVKPTGSAVVGLSMAASSALTLAIYHPQQFVYAGAMSGLLDPSQAMGPTLIGLAMGDAGGYKASDMWGPKEDPAWQRNDPLLNVGKLIANNTRVWVYCGNGKLSDLGGNNLPAKFLEGFVRTSNIKFQDAYNAGGGHNGVFDFPDSGTHSWEYWGAQLNAMKPDLQRGSIPNPLLGLD

SEQ ID NO: 44 - protein, 9 aa, synthetic construct
  REGION 1..9: note = SOURCE - /mol_type="protein" /note="Synthetic peptide corresponding to known immunodominant CD 8+ T cell H-2d restricted epitopes in Ag85A" /organism="Artificial Sequence"
WYDQSGLSV

SEQ ID NO: 45 - protein, 20 aa, synthetic construct
  REGION 1..20: note = SOURCE - /mol_type="protein" /note="Synthetic peptide corresponding to the known immunodominant, CD4+ T cell H-2d restricted epitopes in Ag85A" /organism="Artificial Sequence"
TFLTSELPGWLQANRHVKPT

SEQ ID NO: 46 - DNA, 2274 bp, synthetic construct
  misc_feature 1495..1515: note = /function="Linker"
  misc_feature 1..1494: note = /note="Nucleoprotein sequence"
  misc_feature 1516..2274: note = /note="Matrix protein 1 sequence"
  misc_feature 1..2274: note = source = /mol_type="DNA" /note="Influenza A virus nucleoprotein and matrix protein 1" /organism="Unknown"
  source 1..2274: mol_type = other DNA
  CDS 1..2274
atggccagccagggcaccaagcggagctacgagcagatggaaaccgacggcgaccggcagaacgccaccgagatccgggccagcgtgggcaagatgatcgacggcatcggccggttctacatccagatgtgcaccgagctgaagctgtccgactacgagggccggctgatccagaacagcctgaccatcgagaagatggtgctgtccgccttcgacgagcggcggaacagatacctggaagagcaccccagcgccggcaaggaccccaagaaaaccggcggacccatctaccggcgggtggacggcaagtggatgcgggagctggtgctgtacgacaaagaggaaatccggcggatctggcggcaggccaacaacggcgaggacgccacagccggcctgacccacatgatgatctggcacagcaacctgaacgacaccacctaccagcggaccagggccctcgtgcggaccggcatggacccccggatgtgcagcctgatgcagggcagcacactgcccagaagaagcggagctgccggagccgccgtgaagggcatcggcaccatggtgatggaactgatccggatggtgaagcggggcatcaacgaccggaatttttggaggggcgagaacggcagaaagactagaagcgcctacgagcggatgtgcaacatcctgaagggcaagttccagacagccgcccagcgggccatggtggaccaggtccgggagagccggaaccccggcaacgccgagatcgaggacctgatcttcctggcccggtccgccctgatcctgcggggcagcgtggcccacaagagctgcctgcccgcctgcgtgtacggccctgccgtgagcagcggctacgacttcgagaaagagggctacagcctggtcggcatcgaccccttcaagctgctgcagaacagccaggtgtacagcctgatccggcccaacgagaaccccgcccacaagtcccagctggtctggatggcctgccacagcgccgccttcgaggatctgcggctgctgtccttcatccggggcaccaaggtgtcccccaggggcaagctgtccaccagaggcgtgcagatcgccagcaacgagaacatggacaacatgggcagcagcaccctggaactgcggagcggctactgggccatccggacccggtccggcggcaacaccaaccagcagcgggccagcgccggacagatcagcgtgcagcccaccttctccgtgcagcggaacctgcccttcgagaagagcaccgtgatggccgccttcaccggcaacaccgagggccggaccagcgacatgcgggccgagattatccggatgatggaaggcgccaagcccgaggaagtgagcttccggggcaggggcgtgttcgagctgtccgatgagaaggccaccaaccccatcgtgcccagcttcgagatgagcaacgagggcagctacttcttcggcgacaacgccgaggaatacgacaatggcggcggaccaggcggcggaatgagcctgctgaccgaggtggagacctacgtgctgtccatcgtgcctagcggccctctgaaggccgagatcgcccagcggctggaagatgtgttcgccggcaagaacaccgacctggaagccctgatggaatggctgaaaacccggcccatcctgagccccctgaccaagggcatcctgggcttcgtgttcaccctgaccgtgcccagcgagcggggcctgcagcggcggagattcgtgcagaacgccctgaacggcaacggcgaccccaacaacatggataaggccgtgaagctgtaccggaagctgaagcgggagatcaccttccacggcgccaaagagatcgccctgagctacagcgccggagccctggccagctgcatgggcctgatctacaaccggatgggcgccgtgaccaccgaggtggccttcggcctggtctgcgccacctgcgagcagatcgccgacagccagcacagatcccaccggcagatggtggccacaaccaaccctctgatcaagcacgagaaccggatggtgctggctagcaccaccgccaaggccatggaacagatggccggcagcagcgagcaggccgccgaagccatggaaatcgccagccaggccagacagatggtgcaggccatgcggaccgtgggcacccaccccagcagctccaccggcctgcgggacgacctgctggaaaacctgcagacctaccagaaacggatgggggtgcagatgcagcggttcaagtga

SEQ ID NO: 47 - protein, 757 aa, synthetic construct
  REGION 1..757: note = SOURCE - /mol_type="protein" /note="Influenza A virus nucleoprotein and matrix protein 1" /organism="Unknown"
MASQGTKRSYEQMETDGDRQNATEIRASVGKMIDGIGRFYIQMCTELKLSDYEGRLIQNSLTIEKMVLSAFDERRNRYLEEHPSAGKDPKKTGGPIYRRVDGKWMRELVLYDKEEIRRIWRQANNGEDATAGLTHMMIWHSNLNDTTYQRTRALVRTGMDPRMCSLMQGSTLPRRSGAAGAAVKGIGTMVMELIRMVKRGINDRNFWRGENGRKTRSAYERMCNILKGKFQTAAQRAMVDQVRESRNPGNAEIEDLIFLARSALILRGSVAHKSCLPACVYGPAVSSGYDFEKEGYSLVGIDPFKLLQNSQVYSLIRPNENPAHKSQLVWMACHSAAFEDLRLLSFIRGTKVSPRGKLSTRGVQIASNENMDNMGSSTLELRSGYWAIRTRSGGNTNQQRASAGQISVQPTFSVQRNLPFEKSTVMAAFTGNTEGRTSDMRAEIIRMMEGAKPEEVSFRGRGVFELSDEKATNPIVPSFEMSNEGSYFFGDNAEEYDNGGGPGGGMSLLTEVETYVLSIVPSGPLKAEIAQRLEDVFAGKNTDLEALMEWLKTRPILSPLTKGILGFVFTLTVPSERGLQRRRFVQNALNGNGDPNNMDKAVKLYRKLKREITFHGAKEIALSYSAGALASCMGLIYNRMGAVTTEVAFGLVCATCEQIADSQHRSHRQMVATTNPLIKHENRMVLASTTAKAMEQMAGSSEQAAEAMEIASQARQMVQAMRTVGTHPSSSTGLRDDLLENLQTYQKRMGVQMQRFK

SEQ ID NO: 48 - protein, 9 aa, synthetic construct
  REGION 1..9: note = SOURCE - /mol_type="protein" /note="Synthetic peptide corresponding to known immunodominant CD 8+ T cell H-2d restricted epitope in NP" /organism="Artificial Sequence"
TYQRTRALV

SEQ ID NO: 49 - protein, 9 aa, synthetic construct
  REGION 1..9: note = SOURCE - /mol_type="protein" /note="Linker" /organism="Artificial Sequence"
IPNPLLGLD